SEQUENCE LISTING (19 sequences)

SEQ ID NO: 1 - protein, 384 aa, homo sapiens
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDLWSVGCIMGEMVCHKILFPGRDYIDQWNKVIEQLGTPCPEFMKKLQPTVRTYVENRPKYAGYSFEKLFPDVLFPADSEHNKLKASQARDLLSKMLVIDASKRISVDEALQHPYINVWYDPSEAEAPPPKIPDKQLDEREHTIEEWKELIYKEVMDLEERTKNGVIRGQPSPLAQVQQ

SEQ ID NO: 2 - protein, 331 aa, Homo sapiens
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 3 - protein, 443 aa, Homo sapiens
MADRAEMFSLSTFHSLSPPGCRPPQDISLEEFDDEDLSEITDDCGLGLSYDSDHCEKDSLSLGRSEQPHPICSFQDDFQEFEMIDDNEEEDEEDDEEEEDAEDSAGSPGGRGTGPSAPRDASLVYDAVKYTLVVDEHTQLELVSLRRCAGLGHDSEEDSGGEASEEEAGAALLGGGQVSGDTSPDSPDLTFSKKFLNVFVNSTSRSSSTESFGLFSCLVNGEEREQTHRAVFRFIPRHPDELELDVDDPVLVEAEEDDFWFRGFNMRTGERGVFPAFYAHAVPGPAKDLLGSKRSPCWVERFDVQFLGSVEVPCHQGNGILCAAMQKIATARKLTVHLRPPASCDLEISLRGVKLSLSGGGPEFQRCSHFFQMKNISFCGCHPRNSCYFGFITKHPLLSRFACHVFVSQESMRPVAQSVGRAFLEYYQEHLAYACPTEDIYLE

SEQ ID NO: 4 - protein, 11 aa, artificial sequence
  TI-JIP peptide
RPKRPTTLNLF

SEQ ID NO: 5 - protein, 347 aa, Homo sapiens
METPFYGDEALSGLGGGASGSGGTFASPGRLFPGAPPTAAAGSMMKKDALTLSLSEQVAAALKPAPAPASYPPAADGAPSAAPPDGLLASPDLGLLKLASPELERLIIQSNGLVTTTPTSSQFLYPKVAASEEQEFAEGFVKALEDLHKQNQLGAGRAAAAAAAAAGGPSGTATGSAPPGELAPAAAAPEAPVYANLSSYAGGAGGAGGAATVAFAAEPVPFPPPPPPGALGPPRLAALKDEPQTVPDVPSFGESPPLSPIDMDTQERIKAERKRLRNRIAASKCRKRKLERISRLEEKVKTLKSQNTELASTASLLREQVAQLKQKVLSHVNSGCQLLPQHQVPAY

SEQ ID NO: 6 - protein, 347 aa, Homo sapiens
MCTKMEQPFYHDDSYTATGYGRAPGGLSLHDYKLLKPSLAVNLADPYRSLKAPGARGPGPEGGGGGSYFSGQGSDTGASLKLASSELERLIVPNSNGVITTTPTPPGQYFYPRGGGSGGGAGGAGGGVTEEQEGFADGFVKALDDLHKMNHVTPPNVSLGATGGPPAGPGGVYAGPEPPPVYTNLSSYSPASASSGGAGAAVGTGSSYPTTTISYLPHAPPFAGGHPAQLGLGRGASTFKEEPQTVPEARSRDATPPVSPINMEDQERIKVERKRLRNRLAATKCRKRKLERIARLEDKVKTLKAENAGLSSTAGLLREQVAQLKQKVMTHVSNGCQLLLGVKGHAF

SEQ ID NO: 7 - protein, 487 aa, Homo sapiens
MSDDKPFLCTAPGCGQRFTNEDHLAVHKHKHEMTLKFGPARNDSVIVADQTPTPTRFLKNCEEVGLFNELASPFENEFKKASEDDIKKMPLDLSPLATPIIRSKIEEPSVVETTHQDSPLPHPESTTSDEKEVPLAQTAQPTSAIVRPASLQVPNVLLTSSDSSVIIQQAVPSPTSSTVITQAPSSNRPIVPVPGPFPLLLHLPNGQTMPVAIPASITSSNVHVPAAVPLVRPVTMVPSVPGIPGPSSPQPVQSEAKMRLKAALTQQHPPVTNGDTVKGHGSGLVRTQSEESRPQSLQQPATSTTETPASPAHTTPQTQSTSGRRRRAANEDPDEKRRKFLERNRAAASRCRQKRKVWVQSLEKKAEDLSSLNGQLQSEVTLLRNEVAQLKQLLLAHKDCPVTAMQKKSGYHTADKDDSSEDISVPSSPHTEAIQHSSVSTSNGVSSTSKAEAVATSVLTQMADQSTEPALSQIVMAPSSQSQPSGS

SEQ ID NO: 8 - protein, 351 aa, Homo sapiens
MTEMSFLSSEVLVGDLMSPFDPSGLGAEESLGLLDDYLEVAKHFKPHGFSSDKAKAGSSEWLAVDGLVSPSNNSKEDAFSGTDWMLEKMDLKEFDLDALLGIDDLETMPDDLLTTLDDTCDLFAPLVQETNKQPPQTVNPIGHLPESLTKPDQVAPFTFLQPLPLSPGVLSSTPDHSFSLELGSEVDITEGDRKPDYTAYVAMIPQCIKEEDTPSDNDSGICMSPESYLGSPQHSPSTRGSPNRSLPSPGVLCGSARPKPYDPPGEKMVAAKVKGEKLDKKLKKMEQNKTAATRYRQKKRAEQEALTGECKELEKKNEALKERADSLAKEIQYLKDLIEEVRKARGKKRVP

SEQ ID NO: 9 - protein, 428 aa, Homo sapiens
MDPSVTLWQFLLQLLREQGNGHIISWTSRDGGEFKLVDAEEVARLWGLRKNKTNMNYDKLSRALRYYYDKNIIRKVSGQKFVYKFVSYPEVAGCSTEDCPPQPEVSVTSTMPNVAPAAIHAAPGDTVSGKPGTPKGAGMAGPGGLARSSRNEYMRSGLYSTFTIQSLQPQPPPHPRPAVVLPNAAPAGAAAPPSGSRSTSPSPLEACLEAEEAGLPLQVILTPPEAPNLKSEELNVEPGLGRALPPEVKVEGPKEELEVAGERGFVPETTKAEPEVPPQEGVPARLPAVVMDTAGQAGGHAASSPEISQPQKGRKPRDLELPLSPSLLGGPGPERTPGSGSGSGLQAPGPALTPSLLPTHTLTPVLLTPSSLPPSIHFWSTLSPIAPRSPAKLSFQFPSSGSAQVHIPSISVDGLSTPVVLSPGPQKP

SEQ ID NO: 10 - protein, 551 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLPHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 11 - DNA, 5562 bp, artificial sequence
  pDEATH-TRYP vector
ctagcgattttggtcatgagatcagatcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactccaagcttccccggatcggactactagcagctgtaatacgactcactatagggaatattaagctcaccatgggtaagcctatccctaaccctctcctcggtctcgattctacacaagctatgggtgctcctccaaaaaagaagagaaaggtagctgaattcgagctcagatctcagctgggcccggtaccaattgatgcatcgataccggtactagtcggaccgcatatgcccgggcgtaccgcggccgctcgaggcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcacggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgagagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatga

SEQ ID NO: 12 - DNA, 7551 bp, artificial sequence
  pJFK vector
ccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggaattaagcttatgggtgctcctccaaaaaagaagagaaaggtagctggtatcaataaagatatcgaggagtgcaatgccatcattgagcagtttatcgactacctgcgcaccggacaggagatgccgatggaaatggcggatcaggcgattaacgtggtgccgggcatgacgccgaaaaccattcttcacgccgggccgccgatccagcctgactggctgaaatcgaatggttttcatgaaattgaagcggatgttaacgataccagcctcttgctgagtggagatgcctcctacccttatgatgtgccagattatgcctctcccgaattcggccgactcgagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgttgtaatcgttcttccacacggatcctctagagtcgactagcggccgcttcgacctgcagcaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgatctgtctttcgccgaaacctgtttgatgactacttcatcaattttttttttttctgccgcattccaaaggtcataactttgcaaaaataaagggtaaatggttaaaaattgttatcataaataaggtgaccggttatattgagacctttcctggacagtaactaatacagaagccattggtaatgcaataatttatttgatcatgtgactacgatccgggtgagactattcaaaaaaggagtcaagcattgaaataattaatgactaatccgaagttaattgttaggagtcaattgttttttccaatgaatggaatctgagatgactaaactaccaattttcaatagttcatggtatagtgacgtagttagtgcttttttttcttggatctgttgactcacttcaattgatgtttcttaccctgacatgacatacttgatattttatctctcacgttatataacttgaaaaggatgcacacagttctgttcaatataccctccaatatgtaaaaacagtttttccattgattactcttaatttgtttcctgctaaaccagcagtacgtgtgtgccgtatatattaaaattacactatggtttttgatttgaaaagaattgttagaccaaaaatttataacttggaaccttatcgctgtgcaagagatgatttcaccgagggtatattgctagacgccaatgaaaatgcccatggacctactccagttgaattgagcaagaccaatttacatcgttacccggatcctcaccaattggagttcaagaccgcaatgacgaaatacaggaacaaaacaagcagttatgccaatgacccagaggtaaaacctttaactgctgacaatctgtgcctaggtgtgggatctgatgagagtattgatgctattattagagcatgctgtgttcccgggaaagaaaagattctggttcttccaccaacatattctatgtactctgtttgtgcaaacattaatgatatagaagtcgtccaatgtcctttaactgtttccgacggttcttttcaaatggataccgaagctgtattaaccattttgaaaaacgactcgctaattaagttgatgttcgttacttcaccaggtaatccaaccggagccaaaattaagaccagtttaatcgaaaaggtcttacagaattgggacaatgggttagtcgttgttgatgaagcttacgtagatttttgtggtggctctacagctccactagtcaccaagtatcctaacttggttactttgcaaactctatccaagtcattcggtttagccgggattaggttgggtatgacatatgcaacagcagagttggccagaattttaaatgcaatgaaggcgccttataatatttcctccctagcctctgaatatgcactaaaagctgttcaagacagtaatctaaagaagatggaagccacttcgaaaataatcaatgaagagaaaatgcgcctcttaaaggaattaactgctttggattacgttgatgaccaatatgttggtggattagatgctaattttcttttaatacggatcaacgggggtgacaatgtcttggcaaagaagttatattaccaattggctactcaatctggggttgtcgtcagatttagaggtaacgaattaggctgttccggatgtttgagaattaccgttggaacccatgaggagaacacacatttgataaagtacttcaaggagacgttatataagctggccaatgaataaatagacgtcaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgtgactccccgtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattaattcgagctcggta

SEQ ID NO: 13 - DNA, 7308 bp, artificial sequence
  pDD vector
cttgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcaagggtgcgatatgaaagcgttaacggccaggcaacaagaggtgtttgatctcatccgtgatcacatcagccagacaggtatgccgccgacgcgtgcggaaatcgcgcagcgtttggggttccgttccccaaacgcggctgaagaacatctgaaggcgctggcacgcaaaggcgttattgaaattgtttccggcgcatcacgcgggattcgtctgttgcaggaagaggaagaagggttgccgctggtaggtcgtgtggctgccggtgaaccacttctggcgcaacagcatattgaaggtcattatcaggtcgatccttccttattcaagccgaatgctgatttcctgctgcgcgtcagcgggatgtcgatgaaagatatcggcattatggatggtgacttgctggcagtgcataaaactcaggatgtacgtaacggtcaggtcgttgtcgcacgtattgatgacgaagttaccgttaagcgcctgaaaaaacagggcaataaagtcgaactgttgccagaaaatagcgagtttaaaccaattgtcgtagatcttcgtcagcagagcttcaccattgaagggctggcggttggggttattcgcaacggcgactggctggaattcccggggatccgtcgaccatggcggccgctcgagtcgacctgcagccaagctaattccgggcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgttgtaatccgtccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatgatccgtcgagttcaagagaaaaaaaaagaaaaagcaaaaagaaaaaaggaaagcgcgcctcgttcagaatgacacgtatagaatgatgcattaccttgtcatcttcagtatcatactgttcgtatacatacttactgacattcataggtatacatatatacacatgtatatatatcgtatgctgcagctttaaataatcggtgtcactacataagaacacctttggtggagggaacatcgttggtaccattgggcgaggtggcttctcttatggcaaccgcaagagccttgaacgcactctcactacggtgatgatcattcttgcctcgcagacaatcaacgtggagggtaattctgctagcctctgcaaagctttcaagaaaatgcgggatcatctcgcaagagagatctcctactttctccctttgcaaaccaagttcgacaactgcgtacggcctgttcgaaagatctaccaccgctctggaaagtgcctcatccaaaggcgcaaatcctgatccaaacctttttactccacgcgccagtagggcctctttaaaagcttgaccgagagcaatcccgcagtcttcagtggtgtgatggtcgtctatgtgtaagtcaccaatgcactcaacgattagcgaccagccggaatgcttggccagagcatgtatcatatggtccagaaaccctatacctgtgtggacgttaatcacttgcgattgtgtggcctgttctgctactgcttctgcctctttttctgggaagatcgagtgctctatcgctaggggaccaccctttaaagagatcgcaatctgaatcttggtttcatttgtaatacgctttactagggctttctgctctgtcatctttgccttcgtttatcttgcctgctcattttttagtatattcttcgaagaaatcacattactttatataatgtataattcattatgtgataatgccaatcgctaagaaaaaaaaagagtcatccgctaggtggaaaaaaaaaaatgaaaatcattaccgaggcataaaaaaatatagagtgtactagaggaggccaagagtaatagaaaaagaaaattgcgggaaaggactgtgttatgacttccctgactaatgccgtgttcaaacgatacctggcagtgactcctagcgctcaccaagctcttaaaacggaattatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataaattggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggggtctcgcggtcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattaccccaagctcgaaattaaccctcactaaagggaacaaaagctggtaccgggccccccctcgaaattc

SEQ ID NO: 14 - DNA, 9167 bp, artificial sequence
  pRT2 vector
taccttttgatgcggaattgactttttcgtgaataatacataacttttctgaaaagaatcaaagacagataaaatttaagagatattaaacattagtgagaagccgagaattttgtaacaccaacataacactgacatctttaacaacttttaattatgataaatttcttacgtcatgattgattattacagctatgctgacaaatgactcttgttgcagggctacgaaccgggtaatattaagtgattgactcttgctgaccttttattaagaactaaatggacaatattatggagcatttcatgtataaattggtgcgtaaaatcgttggatctctcttctaagtacatcctactataacaatcaagaaaaacaagaaaaccggacaaaacaatcaagtatggattctagaacagttggtatattgggagggggacaattgggacgtatgattgttgaggcagctaacaggctcaacattaagacggtaatactagatgctgaaaattctcctgccaaacaaataagcaactccaatgaccacgttaatggctccttttccaatcctcttgatatcgaaaaactagctgaaaaatgtgatgtgctaacgattgagattgagcatgttgatgttcctacactaaagaatcttcaagtaaaacatcccaaattaaaaatttacccttctccagaaacaatcggattgatacaagacaaatatattcaaaaagagcatttaatcaaaaatggtatagcagttacccaaagtgtccctgtggaacaagccagtgagacgtccctattgaatgttggaagagatttgggttttccattcgtcttgaagtcgaggactttggcatacgatggaagaggtaacttcgttgtaaagaataaggaaatgattccggaagctttggaagtactgaaggatcgtcctttgtacgccgaaaaatggggaccatttactaaagaattagcagtcatgattgtgagatctgttaacggtttagtgtttttttacccaattgtagagactatccacaaggacaatatttgtgacttatgttatgcgcctgctagagttccggactccgttcaacttaaggcgaagttgttggcgaaaatgcaatcaaacttttcccggttgtggtatattggtgtggaaatgttctatttagaaacaggggaattgcttattaacgaaattgccccaaggcctcacaactctggacattataccattgatgcttgcgtcacttctcaatttgaagctcatttgagatcaatattggatttgccaatgccaaagaatttcacatctttctccaccattacaacgaacgccattatgctaaatgttcttggagacaaacatacaaaagataaagagctagaaacttgcgaaagagcattggcgactccaggttcctcagtgtacttatatggaaaagagtctagacctaacagaaaagtaggtcacataaatattattgcctccagtatggcggaatgtgaacaaaggctgaactacattacagggagaactgatattccactcaaaatctctgtcgctcaaaagttggacttggaagcaatggtcaaaccattggttggagtcatcatgggatcagactctgacttgccggtaatgtctgccgcatgtgcggttttaaaagattttggcgttacatttgaattgacaatagtctctgctcatagaactccacataggatgtcagcatatgctatttccgcaagcaagcgtggaattaaaacaattatcgctggagctggtggggctgctcacttgccaggtatggtggctgcaatgacaccacttcctgtcatcggtgtgcccgtaaaaggttcttgtctagatggagtagattctttacattcaaccgtgcaaatgcctagaggtgttccagtagctaccgtcgctattaataatagtacgaacgctgcgctgttggctgtcagactgcttggcgcttatgattcaagttatacaacaaaaatggaacagtttttattaaagcaggaagaagaagttcttgtcaaagcacaaaagttagaaactgtcggttacgaagcttatctagaaaacaagtaatatataagtttattgatatacttgcacagcaaataatataaaatgatatacctattttttaggctttgttatgattacatcaaatgtggacttcatacatagaaatcaacgcttacaggtgtccttatcgatgctagcttgcatgcctgcagcaattcccgaggctgtagccgacgatggtgcgccaggagagttgttgatcggtactagtcggaccgcatatgcccgggcgtaccgcggccgctcgagtcgacctgcagccaagctaattccgggcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcatgccgagcattatttgtagagctcatccatgccatgtgtaatcccagcagcagttacaaactcaagaaggaccatgtggtcacgcttttcgttgggatctttcgaaagggcagattgtgtcgacaggtaatggttgtctggtaaaaggacagggccatcgccaattggagtattttgttgataatggtctgctagttgaacggatccatcttcaatgttgtggcgaattttgaagttagctttgattccattcttttgtttgtctgccgtgatgtatacattgtgtgagttatagttgtactcgagtttgtgtccgagaatgtttccatcttctttaaaatcaataccttttaactcgatacgattaacaagggtatcaccttcaaacttgacttcagcacgcgtcttgtagttcccgtcatctttgaaagatatagtgcgttcctgtacataaccttcgggcatggcactcttgaaaaagtcatgccgtttcatatgatccggataacgggaaaagcattgaacaccataagagaaagtagtgacaagtgttggccatggaacaggtagttttccagtagtgcaaataaatttaagggtaagctttccgtatgtagcatcaccttcaccctctccactgacagaaaatttgtgcccattaacatcaccatctaattcaacaagaattgggacaactccagtgaaaagttcttctcctttgctagccattctagagaattccgcacttttcggccaatggtcttggtaattcctttgcgctagaattgaactcaggtacaatcacttcttctgaatgagatttagtcattatagttttttctccttgacgttaaagtatagaggtatattaacaattttttgttgatacttttattacatttgaataagaagtaatacaaaccgaaaatgttgaaagtattagttaaagtggttatgcagtttttgcatttatatatctgttaatagatcaaaaatcatcgcttcgctgattaattaccccagaaataaggctaaaaaactaatcgcattatcatccctcgacgtactgtacatataaccactggttttatatacagcagtactgtacatataaccactggttttatatacagcagtcgacgtactgtacatataaccactggttttatatacagcagtactgtacatataaccactggttttatatacagcagtcgaggtaagattagatatggatatgtatatggatatgtatatggtggtaatgccatgtaatatgattattaaacttctttgcgtccatccaaaaaaaaagtaagaatttttgaaaattcaatataaatgacagctcagttacaaagtgaaagtacttctaaaattgttttggttacaggtggtgctggatacattggttcacacactgtggtagagctaattgagaatggatatgactgtgttgttgctgataacctgtcgaattcagatccccgacctgaagtctaggtccctatttatttttttatagttatgttagtattaagaacgttatttatatttcaaatttttcttttttttctgtacagacgcgtgtacgaatttcgacctcgaccgggtaccgagctcggatccccctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttagcttgggacggattacaacaggtattgtcctctgaggacataaaatacacaccgagattcatcaactcattgctggagttagcatatctacaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgatataattcaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccgaattcattcgacaggttatcagcaacaacacagtcatatccattctcaattagctctaccacagtgtgtgaaccaatgtatccagcaccacctgtaaccaaaacaattttagaagtactttcactttgtaactgagctgtcatttatattgaattttcaaaaattcttactttttttttggatggacgcaaagaagtttaataatcatattacatggcattaccaccatatacatatccatatacatatccatatctaatcttacctcgagcattatcaccgccagaggtaaaatagtcaacacgcacggtgttagatatttatcccttgcggtgatagctcgagggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaactataatggaattctgcagccgatgaggaaacccgatgaccaccacactgttgcccggcactgtcaccctcgtcggcgccgggcccggcgaccctgaactcgtcaccgtggccggcctgcgggccgtgcagcaggccgaggtgatcctctacgaccggctcgccccgcaggacctgctgtcggaggcgtccgacgacgccgaactcgtgccggtcggcaagatcccgcgcggccactatgtgccccaggaggagatcaaccaactgctcgtcgcgcacgcccgcgagggccgcaaggtggtgcgcctcaagggtggcgactcgttcgtcttcgggcgtggcggcgaggaatggcaggcctgcgccgaggccggcatcccggtgcgcgtgatcccgggagtctcctcggccaccgcgggcccggcgctggccggcatcccgctgacccatcgccacctggtgcaggggttcaccgtcgtgtcggggcatgtatcgcccagcgacgagcgctccgaggtgccatggcgccaactcgccaaggaccggctcacgctggtgatcctgatgggcgtggcccatatgcgcgacatcgcgccggaattgatggccggcgggctgcctgccgacacccccgtgcgcgtggtgagcaatgcgagcctggccagccaggaatcgtggcgcaccacgctgggcgatgccgtggccgacatggacgcgcaccacgtgcgtccgcccgcgctggtggtggtgggtaccctggccggcgtcgacctgtcgcatcccgaccatcgcgcgcccagcgaccactgagtcgcggccgc

SEQ ID NO: 15 - DNA, 6298 bp, artificial sequence
  pGMS19 plasmid
ggggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaactataaagctgatctaccgtatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttgcaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttgggaatttggaattcgagctcagatctcagctgggcccggtaccgcggccgctcgagtcgacctgcagccaagctaattccgggcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcatgccgagcaaatgcctgcaaatcgctccccatttcacccaattgtctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgtgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcattattttttgctttttctcttgaggtcacatgatcgcaaaatggcaaatggcacgtgaagctgtcgatattggggaactgtggtggttggcaaatgactaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccatacaatgccatctcatttcgatactgttcaactacacgccggccaagagaaccctggtgacaatgctcacagatccagagctgtaccaatttacgccaccacttcttatgttttcgaaaactctaagcatggttcgcaattgtttggtctagaagttccaggttacgtctattcccgtttccaaaacccaaccagtaatgttttggaagaaagaattgctgctttagaaggtggtgctgctgctttggctgtttcctccggtcaagccgctcaaacccttgccatccaaggtttggcacacactggtgacaacatcgtttccacttcttacttatacggtggtacttataaccagttcaaaatctcgttcaaaagatttggtatcgaggctagatttgttgaaggtgacaatccagaagatttcgaaaaggtctttgatgaaagaaccaaggctgtttatttggaaaccattggtaatccaaagtacaatgttccggattttgaaaaaattgttgcaattgctcacaaacacggtattccagttgtcgttgacaacacatttggtgccggtggttacttctgtcagccaattaaatacggtgctgatattgtaacacattctgctaccaaatggattggtggtcatggtactactatcggtggtattattgttgactctggtaagttcccatggaaggactacccagaaaagttccctcaattctctcaacctgccgaaggatatcacggtactatctacaatgaagcctacggtaacttggcatacatcgttcatgttagaactgaactattaagagatttgggtccattgatgaacccatttgcctctttcttgctactacaaggtgttgaaacattatctttgagagctgaaagacacggtgaaaatgcattgaagttagccaaatggttagaacaatccccatacgtatcttgggtttcataccctggtttagcatctcattctcatcatgaaaatgctaagaagtatctatctaacggtttcggtggtgtcttatctttcggtgtaaaagacttaccaaatgccgacaaggaaactgacccattcaaactttctggtgctcaagttgttgacaatttaaagcttgcctctaacttggccaatgttggtgatgccaagaccttagtcattgctccatacttcactacccacaaacaattaaatgacaaagaaaagttggcatctggtgttaccaaggacttaattcgtgtctctgttggtatcgaatttattgatgacattattgcagacttccagcaatcttttgaaactgttttcgctggccaaaaaccatgagtgtgcgtaatgagttgtaaaattatgtataaacatgagatca

SEQ ID NO: 16 - DNA, 12544 bp, artificial sequence
  pDR10 plasmid
ttgcatgcctgcagcaattcccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgtaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacgtaacattctagacattatagttttttctccttgacgttaaagtatagaggtatattaacaattttttgttgatacttttattacatttgaataagaagtaatacaaaccgaaaatgttgaaagtattagttaaagtggttatgcagtttttgcatttatatatctgttaatagatcaaaaatcatcgcttcgctgattaattaccccagaaataaggctaaaaaactaatcgcattatcatccctcgagctatcaccgcaagggataaatatctaacaccgtgcgtgttgactattttacctctggcggtgataatgctcgaggtaagattagatatggatatgtatatggatatgtatatggtggtaatgccatgtaatatgattattaaacttctttgcgtccatccaaaaaaaaagtaagaatttttgaaaattcaatataaatgacagctcagttacaaagtgaaagtacttctaaaattgttttggttacaggtggtgctggatacattggttcacacactgtggtagagctaattgagaatggatatgactgtgttgttgctgataacctgtcgaatagatccccgacctgaagtctaggtccctatttatttttttatagttatgttagtattaagaacgttatttatatttcaaatttttcttttttttctgtacagacgcgtgtacgaatttcgacctcgaccgggtaccgagctcgaggtcagtgcgtacgccatggccggagtggctcacagtcggtggtccggcagtacaacatccaaaagtttgtgttttttaaatagtacataatggatttccttacgcgaaatacgggcagacatggcctgcccggttattattatttttgacaccagaccaactggtaatggtagcgaccggcgctcagctggaattccgccgatactgacgggctccaggagtcgtcgccaccaatccccatatggaaaccgtcgatattcagccatgtgccttcttccgcgtgcagcagatggcgatggctggtttccatcagttgctgttgactgtagcggctgatgttgaactggaagtcgccgcgccactggtgtgggccataattcaattcgcgcgtcccgcagcgcagaccgttttcgctcgggaagacgtacggggtatacatgtctgacaatggcagatcccagcggtcaaaacaggcggcagtaaggcggtcgggatagttttcttgcggccctaatccgagccagtttacccgctctgctacctgcgccagctggcagttcaggccaatccgcgccggatgcggtgtatcgctcgccacttcaacatcaacggtaatcgccatttgaccactaccatcaatccggtaggttttccggctgataaataaggttttcccctgatgctgccacgcgtgagcggtcgtaatcagcaccgcatcagcaagtgtatctgccgtgcactgcaacaacgctgcttcggcctggtaatggcccgccgccttccagcgttcgacccaggcgttagggtcaatgcgggtcgcttcacttacgccaatgtcgttatccagcggtgcacgggtgaactgatcgcgcagcggcgtcagcagttgttttttatcgccaatccacatctgtgaaagaaagcctgactggcggttaaattgccaacgcttattacccagctcgatgcaaaaatccatttcgctggtggtcagatgcgggatggcgtgggacgcggcggggagcgtcacactgaggttttccgccagacgccactgctgccaggcgctgatgtgcccggcttctgaccatgcggtcgcgttcggttgcactacgcgtactgtgagccagagttgcccggcgctctccggctgcggtagttcaggcagttcaatcaactgtttaccttgtggagcgacatccagaggcacttcaccgcttgccagcggcttaccatccagcgccaccatccagtgcaggagctcgttatcgctatgacggaacaggtattcgctggtcacttcgatggtttgcccggataaacggaactggaaaaactgctgctggtgttttgcttccgtcagcgctggatgcggcgtgcggtcggcaaagaccagaccgttcatacagaactggcgatcgttcggcgtatcgccaaaatcaccgccgtaagccgaccacgggttgccgttttcatcatatttaatcagcgactgatccacccagtcccagacgaagccgccctgtaaacggggatactgacgaaacgcctgccagtatttagcgaaaccgccaagactgttacccatcgcgtgggcgtattcgcaaaggatcagcgggcgcgtctctccaggtagcgaaagccattttttgatggaccatttcggcacagccgggaagggctggtcttcatccacgcgcgcgtacatcgggcaaataatatcggtggccgtggtgtcggctccgccgccttcatactgcaccgggcgggaaggatcgacagatttgatccagcgatacagcgcgtcgtgattagcgccgtggcctgattcattccccagcgaccagatgatcacactcgggtgattacgatcgcgctgcaccattcgcgttacgcgttcgctcatcgccggtagccagcgcggatcatcggtcagacgattcattggcaccatgccgtgggtttcaatattggcttcatccaccacatacaggccgtagcggtcgcacagcgtgtaccacagcggatggttcggataatgcgaacagcgcacggcgttaaagttgttctgcttcatcagcaggatatcctgcaccatcgtctgctcatccatgacctgaccatgcagaggatgatgctcgtgacggttaacgcctcgaatcagcaacggcttgccgttcagcagcagcagaccattttcaatccgcacctcgcggaaaccgacatcgcaggcttctgcttcaatcagcgtgccgtcggcggtgtgcagttcaaccaccgcacgatagagattcgggatttcggcgctccacagtttcgggttttcgacgttcagacgtagtgtgacgcgatcggcataaccaccacgctcatcgataatttcaccgccgaaaggcgcggtgccgctggcgacctgcgtttcaccctgccataaagaaactgttacccgtaggtagtcacgcaactcgccgcacatctgaacttcagcctccagtacagcgcggctgaaatcatcattaaagcgagtggcaacatggaaatcgctgatttgtgtagtcggtttatgcagcaacgagacgtcacggaaaatgccgctcatccgccacatatcctgatcttccagataactgccgtcactccaacgcagcaccatcaccgcgaggcggttttctccggcgcgtaaaaatgcgctcaggtcaaattcagacggcaaacgactgtcctggccgtaaccgacccagcgcccgttgcaccacagatgaaacgccgagttaacgccatcaaaaataattcgcgtctggccttcctgtagccagctttcatcaacattaaatgtgagcgagtaacaacccgtcggattctccgtgggaacaaacggcggattgaccgtaatgggataggttacgttggtgtagatgggcgcatcgtaaccgtgcatctgccagtttgaggggacgacgacagtatcggcctcaggaagatcgcactccagccagctttccggcaccgcttctggtgccggaaaccaggcaaagcgccattcgccattcaggctgcgcaactgttgggaagggcgatcggtgcgggcctcttcgctattacgccagctggcgaaagggggatgtgctgcaaggcgattaagtcgggaaacctgtcgtgccagctgcattaatgaatcggccaacgcgcggggagaggcggtttgcgtattgggcgccagggtggtttttcttttcaccagtgagacgggcaacagccaagctccggatccgggcttggccaagcttggaattccgcacttttcggccaatggtcttggtaattcctttgcgctagaattgaactcaggtacaatcacttcttctgaatgagatttagtcattatagttttttctccttgacgttaaagtatagaggtatattaacaattttttgttgatacttttattacatttgaataagaagtaatacaaaccgaaaatgttgaaagtattagttaaagtggttatgcagtttttgcatttatatatctgttaatagatcaaaaatcatcgcttcgctgattaattaccccagaaataaggctaaaaaactaatcgcattatcatccctcgacgtactgtacatataaccactggttttatatacagcagtactgtacatataaccactggttttatatacagcagtcgacgtactgtacatataaccactggttttatatacagcagtactgtacatataaccactggttttatatacagcagtcgaggtaagattagatatggatatgtatatggatatgtatatggtggtaatgccatgtaatatgattattaaacttctttgcgtccatccaaaaaaaaagtaagaatttttgaaaattcaatataaatgacagctcagttacaaagtgaaagtacttctaaaattgttttggttacaggtggtgctggatacattggttcacacactgtggtagagctaattgagaatggatatgactgtgttgttgctgataacctgtcgaattcgatccccctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttagcttgggacggattacaacaggtattgtcctctgaggacataaaatacacaccgagattcatcaactcattgctggagttagcatatctacaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgatataattcaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcgggactgtagagaccacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggactagtgatctctcttctaagtacatcctactataacaatcaagaaaaacaagaaaatcggacaaaacaatcaagtatggattctagaacagttggtatattaggagggggacaattgggacgtatgattgttgaggcagcaaacaggctcaacattaagacggtaatactagatgctgaaaattctcctgccaaacaaataagcaactccaatgaccacgttaatggctccttttccaatcctcttgatatcgaaaaactagctgaaaaatgtgatgtgctaacgattgagattgagcatgttgatgttcctacactaaagaatcttcaagtaaaacatcccaaattaaaaatttacccttctccagaaacaatcagattgatacaagacaaatatattcaaaaagagcatttaatcaaaaatggtatagcagttacccaaagtgttcctgtggaacaagccagtgagacgtccctattgaatgttggaagagatttgggttttccattcgtcttgaagtcgaggactttggcatacgatggaagaggtaacttcgttgtaaagaataaggaaatgattccggaagctttggaagtactgaaggatcgtcctttgtacgccgaaaaatgggcaccatttactaaagaattagcagtcatgattgtgagatctgttaacggtttagtgttttcttacccaattgtagagactatccacaaggacaatatttgtgacttatgttatgcgcctgctagagttccggactccgttcaacttaaggcgaagttgttggcagaaaatgcaatcaaatcttttcccggttgtggtatatttggtgtggaaatgttctatttagaaacaggggaattgcttattaacgaaattgccccaaggcctcacaactctggacattataccattgatgcttgcgtcacttctcaatttgaagctcatttgagatcaatattggatttgccaatgccaaagaatttcacatctttctccaccattacaacgaacgccattatgctaaatgttcttggagacaaacatacaaaagataaagagctagaaacttgcgaaagagcattggcgactccaggttcctcagtgtacttatatggaaaagagtctagacctaacagaaaagtaggtcacataaatattattgcctccagtatggcggaatgtgaacaaaggctgaactacattacaggtagaactgatattccaatcaaaatctctgtcgctcaaaagttggacttggaagcaatggtcaaaccattggttggaatcatcatgggatcagactctgacttgccggtaatgtctgccgcatgtgcggttttaaaagattttggcgttccatttgaagtgacaatagtctctgctcatagaactccacataggatgtcagcatatgctatttccgcaagcaagcgtggaattaaaacaattatcgctggagctggtggggctgctcacttgccaggtatggtggctgcaatgacaccacttcctgtcatcggtgtgcccgtaaaaggttcttgtctagatggagtagattctttacattcaattgtgcaaatgcctagaggtgttccagtagctaccgtcgctattaataatagtacgaacgctgcgctgttggctgtcagactgcttggcgcttatgattcaagttatacaacgaaaatggaacagtttttattaaagcaagaagaagaagttcttgtcaaagcacaaaagttagaaactgtcggttacgaagcttatctagaaaacaagtaatatataagtttattgatatacttgtacagcaaataattataaaatgatatacctattttttaggctttgttatgattacatcaaatgtggacttcatacatagaaatcaacgcttacaggtgtccttatcgatgctagc

SEQ ID NO: 17 - protein, 311 aa, Homo sapiens
MAALRYAGLDDTDSEDELPPGWEERTTKDGWVYYANHTEEKTQWEHPKTGKRKRVAGDLPYGWEQETDENGQVFFVDHINKRTTYLDPRLAFTVDDNPTKPTTRQRYDGSTTAMEILQGRDFTGKVVVVTGANSGIATGSCHHRVLCCCPRTGGSGRDVLQQLLPLHALTRSSERRDGPDPVGAQREADPRTAWQPVRLSGAQSGWAHTPALCVSPHASARAGPLPNVPPTQIRKSKGNKSSHNRVKNLKYQWEAGNSWGKVSLFWGWARHRSLCFLVVACLKVKTCLVCRFRISLEKHQQFSFFYCYRIA

SEQ ID NO: 18 - protein, 414 aa, Homo sapiens
MAALRYAGLDDTDSEDELPPGWEERTTKDGWVYYANHTEEKTQWEHPKTGKRKRVAGDLPYGWEQETDENGQVFFVDHINKRTTYLDPRLAFTVDDNPTKPTTRQRYDGSTTAMEILQGRDFTGKVVVVTGANSGIGFETAKSFALHGAHVILACRNMARASEAVSRILEEWHKAKVEAMTLDLALLRSVQHFAEAFKAKNVPLHVLVCNAATFALPWSLTKDGLETTFQVNHLGHFYLVQLLQDVLCRSAPARVIVVSSESHRFTDINDSLGKLDFSRLSPTKNDYWAMLAYNRSKLCNILFSNELHRRLSPRGVTSNAVHPGNMMYSNIHRSWWVYTLLFTLARPFTKSMQQGAATTVYCAAVPELEGLGGMYFNNCCRCMPSPEAQSEETARTLWALSERLIQERLGSQSG

SEQ ID NO: 19 - protein, 414 aa, Mus musculus
MAALRYAGLDDTDSEDELPPGWEERTTKDGWVYYANHTEEKTQWEHPKTGKRKRVAGDLPYGWEQETDENGQVFFVDHINKRTTYLDPRLAFTVDDNPTKPTTRQRYDGSTTAMEILQGRDFTGKVVLVTGANSGIGFETAKSFALHGAHVILACRNLSRASEAVSRILEEWHKAKVEAMTLDLAVLRSVQHFAEAFKAKNVSLHVLVCNAGTFALPWGLTKDGLETTFQVNHLGHFYLVQLLQDVLCRSSPARVIVVSSESHRFTDINDSSGKLDLSRLSPPRSDYWAMLAYNRSKLCNILFSNELHRRLSPRGVTSNAVHPGNMMYSAIHRNSWVYKLLFTLARPFTKSMQQGAATTVYCAVAPELEGLGGMYFNNCCRCLPSEEAQSEETARALWELSERLIQDRLGSPSS

(19) United States
(12) Patent Application Publication
(10) Pub. No.: US 2008/0044815 A1
(43) Pub. Date: Feb. 21, 2008

(54) Genetic Screen for Interaction Interface Mapping

(76) Inventors: Paul Michael Watt, Mount Claremont (AU); Richard Hopkins, North Perth (AU); Marie Bogoyevitch, Innaloo (AU)

Correspondence Address: COZEN O'CONNOR, P.C., 1900 MARKET STREET, PHILADELPHIA, PA, 19103-3508, US

(21) Appl. No.: 10/558,863

(22) PCT Filed: May 31, 2004
(86) PCT No.: PCT/AU04/00723

§ 371 Date: Apr. 12, 2006

(30) Foreign Application Priority Data
May 30, 2003 (US) 60474465

(51) Int. Cl. C12Q 1/68

(52) U.S. Cl. 435/6

(57) ABSTRACT

The present invention provides improved reverse hybrid assay methods for identifying amino acid residues within a protein that are required for its interaction or physical association with another protein, wherein disruption of an interaction between a protein of interest and its binding partner protein is assayed for a library of mutations of said protein of interest, and maintenance of an interaction between the protein of interest and another binding partner is assayed simultaneously in a single step, thereby reducing the incidence of uninformative mutations in the protein of interest that are detected.

### FIELD OF THE INVENTION

The present invention relates generally to methods for identifying and/or characterizing and/or isolating the binding domain or binding site and/or one or more specific amino acid residues within a protein that are required for the interaction or physical association of that protein with another protein. More particularly, the present invention provides a method for identifying a region in a protein of interest that mediates the ability of the protein to bind to a binding partner protein in a protein complex in vitro or in vivo. The invention also provides the means for producing highly specific inhibitory peptides (ie., peptide antagonists) that comprise an amino acid sequence of the native binding domain or binding site. The invention also encompasses isolated peptides comprising an amino acid sequence corresponding to the binding domain or binding site determined by the inventive method to be required for the interaction or physical association of one protein with another protein. The invention also provides a method for determining a mutation that disrupts the interaction between two or more proteins such as, for example, by affecting an allosteric change in the conformation of one of the binding partners. The invention further encompasses processes of rational drug design for inhibitors of protein-protein interactions comprising the method of the invention, and small molecule inhibitors that mimic the effects of the inhibitory peptides of the invention.

### BACKGROUND OF THE INVENTION

### 1. General Information

This specification contains nucleotide and amino acid sequence information prepared using PatentIn Version 3.1, presented herein after the claims. Each nucleotide sequence, is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (eg. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, microbiology, virology, recombinant. DNA technology, peptide synthesis in solution, solid phase peptide synthesis, and immunology. Such procedures are described, for example, in the following texts:
1. Sambrook, Fritsch & Maniatis, whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Animal Cell Culture: Practical Approach, Third Edition (John R. W. Masters, ed., 2000), ISBN 0199637970, whole of text;
6. Immobilized Cells and Enzymes: A Practical Approach (1986) IRL Press, Oxford, whole of text;
7. Perbal, B. A Practical Guide to Molecular Cloning (1984);
8. Methods In Enzymology (S. Colowick and N. Kaplan, eds., Academic Press, Inc.), whole of series;
9. J. F. Ramalho Ortigão, “The Chemistry of Peptide Synthesis” In: Knowledge database of Access to Virtual Laboratory website (Interactiva, Germany);
10. Sakakibara, D., Teichman, J., Lien, E. Land Fenichel, R. L. (1976). Biochem. Biophys. Res. Commun. 73 336-342
11. Merrifield, R. B. (1963). J. Am. Chem. Soc. 85, 2149-2154.
12. Barany, G. and Merrifield, R. B. (1979) in The Peptides (Gross, E. and Meienhofer, J. eds.), vol. 2, pp. 1-284, Academic Press, New York.
13. Wünsch, E., ed. (1974) Synthese von Peptiden Houben-Weyls Metoden der Organischen Chemie (Müler, E., ed.), vol. 15, 4th edn., Parts 1 and 2, Thieme, Stuttgart.
14. Bodanszky, M. (1984) Principles of Peptide Synthesis, Springer-Verlag, Heidelberg.
15. Bodanszky, M. & Bodanszky, A. (1984) The Practice of Peptide Synthesis, Springer-Verlag, Heidelberg.
16. Bodanszky, M. (1985) Int. J. Pepfide Protein Res. 25, 449-474.
17. Handbook of Experimental Immunology, Vols. I-IV (D. M. Weir and C. C. Blackwell, eds., 1986, Blackwell Scientific Publications).
18. McPherson et al., In: PCR A Practical Approach., IRL Press, Oxford University Press, Oxford, United Kingdom, 1991.
19. Methods in Yeast Genetics: A Cold Spring Harbor Laboratory Course Manual (D. Burke et al., eds) Cold Spring Harbor Press, New York, 2000 (see whole of text).
20. Guide to Yeast Genetics and Molecular Biology. In: Methods in Enzymology Series, Vol. 194 (C. Guthrie and G. R. Fink eds) Academic Press, London, 1991 2000 (see whole of text).

### 2. Description of the Related Art

Protein-protein interactions are involved in a wide variety of processes occurring in living cells, such as, for example, gene expression, cellular differentiation, growth, enzyme activity, metabolite flow, or metabolite partitioning between cellular compartments. Many of the proteins involved in these interactions are involved in numerous different interactions that may occur, simultaneously in the cell, or alternatively, occur under predefined environmental or developmental conditions. Accordingly, such proteins may form branch-points in signal transduction pathways, which will be known to those skilled in the art of biochemistry to be potential or actual regulatory control points.

For example, three parallel mitogen activated protein (MAP) kinase pathways (i.e., p38, SAPK/JNK and ERK) converge to mediate effects of pro-inflammatory cytokines in different organ systems, including the brain (FIG. 1). Members of c-Jun N-terminal kinase (JNK) family act as an integration point for multiple intracellular biochemical signals governing a wide variety of cellular processes such as proliferation, differentiation, apoptosis, migration, transcriptional regulation, and development. JNK targets specific transcription factors and thus mediates immediate-early gene expression in response to various stress signals including ultraviolet (UV) radiation, oxidative stress, aberrant protein folding in endoplasmic reticulum, osmotic shock, and inflammatory mediators. These transcription factors include ATF-2, Elk1, CREB, NF-kappaB, and AP1 family proteins, such as, for example, p53, JunD, JunB, c-Jun, v-Jun, and Fas (Whitemarsh et al., J. Mol. Med. 74, 589-607, 1996; Angel and Karin, Biochim. Biophys. Acta 1072, 129-157, 1991). Several upstream dual specific protein kinases, such as MKK4/SEK1 and MKK7, can activate JNK through phosphorylation of the conversed Thr-Pro-Tyr motif on JNK proteins. In mammalian cells, activated JNK can phosphorylate the N-terminus of c-Jun, which contains both JNK docking site and JNK phosphorylation site (ser63 and ser73), or JunD, which lacks a JNK docking site but contains a INK phosphorylation site. JNK is unable to phosphorylate JunB due to the lack of a JNK phosphorylation site in JunB, despite the presence of a functional JNK docking site. Comparison of the binding activity of JNK isoforms demonstrates that JNK2 binds c-Jun approximately 25 times more efficiently than JNK1. Therefore, individual members of the JNK family may selectively target specific transcription factors in vivo.

One of the most important functions of JNK is the regulation of apoptosis. Emerging evidence indicates that JNK activation is obligatory for apoptosis induced by a receptor-mediated extrinsic pathway and/or a mitochondria-mediated intrinsic pathway. JNK activation may contribute to the initiation of Fas-induced apoptosis, possibly through the amplification of autocrine or paracrine Fas signaling by JNK-dependent Fas ligand (FasL) gene expression. In addition, JNK has been implicated in apoptosis that is induced by Daxx, a Fas death domain (FADD) interaction protein. Through its serine/threonine kinase activity, JNK may contribute to mitochondria-mediated apoptosis by phosphorylating pro-apoptotic or anti-apoptotic Bcl-2 family proteins, eg., BIM. Finally, JNK has also been indicated as an important kinase phosphorylating p53 and subsequently facilitating p53-dependent apoptotic responses.

In an animal model of neuronal apoptosis arising from stroke (loss or reduced blood supply to the brain), Herdegen et al., J. Neurosci 18, 124-135, 1998 showed that apoptotic neurons have enhanced phosphorylation of the transcription factor c-Jun. Similarly, a non-phosphorylatable c-Jun mutant protein has been shown to promote neuronal survival (Whitfield et al., Neuron 29, 629-643, 2001). Similar effects are observed in models of Alzheimer's disease and Parkinson's disease. Because c-Jun N-terminal kinase proteins (“SAPK” or “JNK proteins) are the primary regulators of c-Jun phosphorylation (Hibi et al., Genes Dev. 7, 2135-2148, 1993), the JNK proteins are thought to be important regulatory proteins in neuronal cell death via interactions with c-Jun proteins. This hypothesis is supported by the ability of the JNK inhibitor CEP-1347 (Cephalon) to support the survival of embryonic neurons (Borasio et al., Neuroreport 9, 1435-1439, 1998), attenuate the loss of neurons in vivo (Saporito et al., J. Pharmacol. Exp. Ther. 288, 421-427, 1999), and preserve the metabolism and growth of nerve growth factor (NGF)-deprived neurons (Harris et al., J. Neurosci 22, 103-113, 2002). Cytochrome c release is an important event in neuronal apoptosis, because it is required for the activation of effector caspases, and it is believed that c-Jun regulates the expression of genes that control cytochrome c release, such as, for example, a pro-apoptotic Bcl-2-like protein designated “BIM”, in neurons deprived of NGF.

In another example, the GTPases Ras and Krev-1 are 56% identical and are known to interact with an overlapping set of protein partners, albeit at different affinities, namely, Raf to which Ras preferentially binds, Krit-1 to which Krev-1 preferentially binds, and the Ral guanine dissociation stimulator protein (RalGDS), to which both proteins bind (Serebriiskii et al., J. Biol. Chem. 274, 17080-17087, 1999). Similarly, the transcription factor SCL, which is, expressed in malignant lymphoid cells, interacts with LMO1, LMO2, DRG, mSin3A, and E47 proteins (Mahajan et al, Oncogene 12, 2343, 1996).

In consideration of this complexity of protein-protein interactions that occurs in vivo, the difficulty associated with modulating the activity of a specific protein or signalling pathway is achieving specificity. For example, in the amelioration or treatment of a disease state that is directly or indirectly caused by aberrant association of cJun with a JNK protein, it is important to avoid undesirable side-effects produced by modulation of a linked pathway involving either or both protein partners.

Accordingly, there is a need to develop highly-specific peptides that modulate the ability of a first protein to bind to or interact physically with a second protein without adversely affecting the ability of the first protein to bind to a protein other than the second protein in a cell and/or in vivo. Peptides comprising a binding site of the first protein to the second protein, or at least consisting of or comprising an amino acid sequence that includes one or more residues essential for binding of the first protein to the second protein, are clearly useful as highly specific antagonists. Such peptides can be used as dominant negative inhibitors or to validate prospective drug targets, by observing a phenotype that results from over-expressing the peptide in ex-vivo assays or in transgenic animal (eg., mouse) models of a disease or condition. Alternatively, or in addition, such peptides are useful for designing peptide mimetic compounds (herein “phylomers” eg., WO00/68373 incorporated herein in its entirety by reference) and non-peptide mimetic compounds.

It is known to identify the interaction site between a protein and its ligand by analysing peptide fragments that have been generated following covalent attachment of the labelled ligand. However, in the case of protein ligands, the process does not necessarily permit fine structure mapping and is susceptible to steric hindrance of proteolysis by the protein complex formed.

Alternative methods known in the art require an analysis of the ability of one or a panel of mutants of one protein to interact with the other wild-type protein and then determining those mutants wherein the interaction is partially or completely abrogated. In general, such methods require additional process steps to clearly distinguish non-informative mutations that affect protein stability, folding or activity generally from those mutations that are limited to the binding site. Identification of the binding site is often based upon the screening of a sufficiently large panel of mutants and identifying those mutations that are clustered within a region of the protein of interest. Such clustered mutations may be deemed informative merely based upon their presence within a conserved domain of the protein, which may not necessarily be indicative of function.

For example, Vidal (WO 96/32503) described a two-step selection method based upon a reverse hybrid screening approach, to identify residues in E2F1 which mediate its ability to interact with DP1. Reverse hybrid screening methods are described in detail in WO99/35282 and WO01/66787, both of which are incorporated herein by reference in their entirety. The two-step method of Vidal requires the identification of mutations that adversely affect the ability of DP1 and E2F1 to bind to each other, and, in a second step, the identification of mutations that, do not completely abrogate the interaction between the proteins. This strategy was based on the premise that mutations that completely destroy the ability of E2F1 to interact with DP1 may represent uninformative mutations, such as those that alter the size or native conformation of the protein (e.g., nonsense mutations, deletions, or insertions). By subtracting those mutations that completely abrogate the interaction from that that do not, a pool of mutations is obtained that comprises mutations wherein the binding site is mutated. However, a significant number of the mutations obtained by this method will comprise uninformative mutations outside the binding site. This method is also limited to facilitating the identification of alleles (e.g., alleles selected from a library of alleles) that only mildly affect the protein/protein interaction, since the method is predicated on the assumption that strong mutations are uninformative. In the example described by Vidal (WO 96/32503) expression of a GAL1:HIS3 reporter gene (Durfee et al., Genes & Dev. 7, 555-569, 1993), was operably linked to the E2F1/DP1 association, such that cells in which GAL:HIS3 was expressed grew on a medium lacking histidine and containing high concentrations of 3AT. The authors identified 12 mutant alleles in E2F1, and in 11 of these 12 alleles, a single nucleotide change in the 1.2 kb nucleotide sequence encoding E2F1 was detected. However, only 6 of the mutations mapped to a putative binding domain required for the E2F1/DP1 association.

Knapp et al., Oncogene 19, 4706-4712, 2000, used a reverse two-hybrid method to identify JunD mutants that do not interact with menin. In this case, the authors merely looked for mutations that completely abrogated the interaction and then performed a second selection to identify those mutants that expressed a JunD protein having the length of the native protein. As with the method described by Vidal, Knapp et al found it necessary to manually select and discard clones that contained nonsense mutations.

Furthermore in this case, the folding of the mutant protein was not studied. Accordingly, this study did not identify or select against mutations that affect large allosteric changes in JunD folding.

Thus, the prior art methods for identifying a site of interaction between two proteins are time-consuming and produce” a relatively high proportion of false positives.

Accordingly, there remains a need for improved methods to identify the site of a protein that interacts with another protein.

### SUMMARY OF THE INVENTION

In work leading up to the present invention, the inventors sought to produce improved methods for the rapid identification of a site of interaction between two proteins. They reasoned that the number of false positives identified using reverse hybrid screening approaches could be minimized or significantly reduced by providing an internal control to the screening process that excluded many or most uninformative mutations, such as those that alter the size or native conformation of the protein (e.g., nonsense mutations, deletions, or insertions) whilst permitting the simultaneous identification of informative mutations.

More particularly, the present inventors reasoned that they could achieve this reduction in uninformative mutations if they included in the screen an internal control for protein conformation or, function, in particular by simultaneously monitoring the protein of interest for its ability to bind to two or more protein partners in a single screen and selecting those mutations that merely abrogate binding of the protein of interest to one protein partner. Preferably, the binding partners for the protein of interest are selected such that they do not compete with each other for binding to the protein of interest or otherwise squelch expression of a reporter molecule or sterically hinder each, other's activation of reporter gene expression. By ensuring that the protein of interest is capable of binding to other protein partners in the screen, improperly folded or truncated proteins are less likely to be selected.

Furthermore, the present invention provides a method of identifying a mutation in a protein that causes an allosteric change in said protein. Accordingly, the screening process used in the present invention is modified to identify but not select against such mutations.

Higher order reverse hybrid screens are used to express dual bait proteins in a cell, such as, for example, a first bait protein selected from the group consisting of an AP-1 family protein (eg., p53, JunD, JunB, c-Jun, v-Jun, or Fas), and a fragment of an AP-1 family protein that interacts with JNK (SEQ ID NO: 1), and a second bait protein selected from the group consisting ATF-2, Elk1, CREB, NF-kappaB, a WOX protein, a fragment of ATF-2 that interacts with JNK, a fragment of Elk1 that interacts with JNK, a fragment of CREB that interacts with JNK, a fragment of NF-kappaB that interacts with JNK and a fragment of a WOX protein that interacts with JNK. Each bait protein is expressed as a fusion protein with the DNA binding domain or the activation domain of a transcription factor, as in standard reverse hybrid screens described in the art. A prey comprising a mutant or variant JNK protein is also expressed in the same cell as a fusion protein with the DNA binding domain or the activation domain of a transcription factor, as in standard reverse hybrid screens, such that the binding of the first and/or second bait protein to the prey reconstitutes a functional transcription factor. The binding of the prey to the first and second bait proteins activates the expression of distinct reporter genes, wherein the interaction of interest is operably linked to the expression of a counter-selectable reporter that can inhibit/reduce cell growth or viability. Cells are then selected under appropriate screening conditions wherein the expression of the counter selectable reporter gene alone is reduced or inhibited, and the expression of the other reporter gene (i.e. linked to the association between. the prey and the other bait) is not abrogated or reduced.

Accordingly, one aspect of the present invention provides a method for identifying a region in a protein of interest that mediates the ability of the protein to bind to a binding partner protein in a protein complex that comprises more than two proteins, said method comprising expressing a mutated form of the protein of interest and the native form of the binding partner protein and native forms of one or more other proteins that bind to the protein of interest such that the binding of the mutated form of the protein of interest to the native form of the binding partner protein to each other protein operably and separately controls the expression of a different reporter gene, and selecting for modified expression of the reporter gene that is operably under the control of a binding between the protein of interest and the binding partner protein and unmodified expression of each other reporter gene, wherein said modified expression indicates that the mutation is within a region in the protein of interest that mediates the ability of the protein to bind to the binding partner protein.

Preferably, the unmodified expression of a reporter gene consists of about the same level of expression of said reporter gene in the presence of a native form of the protein of interest and a native form of the other protein.

Alternatively, or in addition, the modified expression consists of a reduced expression of a reporter gene relative to the expression of the reporter gene in the presence of a native form of the protein of interest and a native form of the binding partner protein.

In one embodiment, reduced expression of the reporter gene is determined in a forward hybrid assay wherein binding between the protein of interest and the binding partner activates expression of a reporter gene and wherein reduced expression of the reporter gene indicates that a mutation in the mutated form of the protein of interest is within a region of the protein of interest that mediates the ability of the protein of interest to bind to the binding partner protein. In accordance with this embodiment, the reporter gene may encode a detectable protein such as a fluorescent protein (e.g., a green fluorescent protein (GFP), luciferase protein, or a product of the cobA gene) or a colored protein that can be detected colorimetrically (e.g., lacZ protein or β-galactosidase), or an antigenic protein that can be detected immunologically by antibody binding to the protein (e.g., a FLAG epitope), or a protein that can be detected enzymatically. Preferably, one or more of the reporter genes encodes a protein that can be detected by fluorometric or colorometric means such that the relative activation of reporter genes can be monitored and/or selected using high throughput techniques such as FACS sorting.

In an alternative embodiment, the reduced expression of the reporter gene is determined in a reverse hybrid assay wherein binding between the protein of interest and the binding partner activates expression of a counter selectable reporter gene encoding a polypeptide that is capable of reducing cell growth or viability by providing a target for a cytotoxic or cytostatic product or by converting a substrate to a cytotoxic or cytostatic product and wherein reduced expression of the counter selectable reporter gene enhances cell growth or viability thereby indicating that a mutation in the mutated form of the protein of interest is within a region of the protein of interest that mediates the ability of the protein of interest to bind to the binding partner protein. In accordance with this embodiment, the counter selectable reporter gene is preferably selected from the group consisting of URA3, CYH2, and LYS2.

Other suitable reporter genes for performing the invention described herein are selected from the group consisting of tet^{r}, Amp^{r}, Rif^{r}, bsdf^{r}, zeof^{r}, Kan^{r}, g, cobA, LacZ, CYH2, TRP1, LYS2, HIS3, HIS5, LEU2, URA3, ADE2, MET13 and MET15.

The protein of interest and the binding partner protein can be the same protein (i.e., in an assay for homodimer formation) or allelic variants of the same protein, or different proteins altogether. Similarly, the binding partner protein and other protein can be allelic variants or mutant forms or orthologues of the same protein.

In accordance with the foregoing embodiments, it is particularly preferred for the protein of interest and/or the protein binding partner and/or the other proteins is/are expressed as one or more fusion protein(s).

Preferably, the protein of interest, the protein binding partner and the other proteins are each expressed as a fusion protein. In one embodiment, a fusion protein comprising the binding partner fusion comprises a DNA binding domain; and a fusion protein comprising said other protein comprises a DNA binding domain such that binding between the protein of interest and the binding partner protein permits binding to the 5′-UTR of a reporter gene thereby activating its expression and binding between the protein of interest and said other protein permits binding to the 5′-UTR of a reporter gene thereby activating its expression. In an alternative embodiment, the fusion protein comprising the protein of interest comprises the transcription activation domain of a transcription factor; the fusion protein comprising the binding partner fusion comprises a DNA binding domain; and (iii) a fusion protein comprising said other protein comprises a DNA binding domain such that binding between the protein of interest and the binding partner protein permits binding to the 5′-UTR of a reporter gene thereby activating its expression and binding between the protein of interest and said other protein permits binding to the 5′-UTR of a reporter gene thereby activating its expression.

Any protein interactions are capable of being assayed in the method of the present invention. In one preferred embodiment, the protein of interest is an oncoprotein SCL or a dimerization region of SCL or a fusion protein comprising said SCL or said dimerization region of SCL and a transcriptional activation domain of a transcription factor; the protein binding partner and other protein are selected from the group consisting of: LMO1, LMO2, DRG, mSin3A, E47, a dimerization region of LMO1, a dimerization region of LMO2, a dimerization region of DRG, a dimerization region of mSin3A, a dimerization region of E47, a fusion protein comprising LMO1, LMO2, DRG, mSin3A or E47 fused to a DNA binding domain, and a fusion protein comprising a dimerization region of LMO1, LMO2, DRG, mSin3A or E47 fused to a DNA binding domain.

In a particularly preferred embodiment, the protein of interest is a MAP kinase protein or a fragment thereof or a fusion protein comprising said MAP kinase protein or said fragment fused to a transcription activation domain. More preferably, the MAP kinase is selected from the group consisting of a p38, a fragment of p38, stress-activated protein kinase (SAPK), a fragment of SAPK, JNK, a fragment of JNK, extracellular regulated protein kinase (ERK) and a fragment of ERK. In accordance with this embodiment, the JNK protein may comprise an amino acid sequence that is at least about 70% identical to the sequence set forth in SEQ ID NO: 1.

Preferred fragments of JNK comprise at least about 5 contiguous amino acids of SEQ ID NO: 1 sufficient to bind to one or more proteins selected from the group consisting of c-Jun (SEQ ID NO: 2), JIP2 (SEQ ID NO: 3), JunD (SEQ ID NO: 5), JunB (SEQ ID NO: 6), ATF-2 (SEQ ID NO: 7), CREB2 (SEQ ID NO: 8), Elk1 (SEQ ID NO: 9), NF-kappaB (SEQ ID NO: 10), human WOX3 (SEQ ID NO: 17), human WOX1 (SEQ ID NO: 18) and murine WOX1 (SEQ ID NO: 19).

Preferred fusion proteins comprising a JNK protein or a fragment thereof are fused to the activation domain of a transcription factor. Thus, preferred fusion proteins comprise at least about 5 contiguous amino acids of SEQ ID NO: 1 sufficient to bind to one or more proteins selected from the group consisting of c-Jun (SEQ ID NO: 2), JIP2 (SEQ ID NO: 3), JunD (SEQ ID NO: 5), JunB (SEQ ID NO: 6), ATF-2 (SEQ ID NO: 7), CREB2 (SEQ ID NO: 8), Elk1 (SEQ ID NO: 9), NF-kappaB (SEQ ID NO: 10), human WOX3 (SEQ ID NO: 17), human WOX1 (SEQ ID NO: 18) and murine WOX1 (SEQ ID NO: 19) fused to the activation domain of a transcription factor.

In a related embodiment, the protein of interest is a JNK protein or fragment thereof sufficient to bind to an AP-1 family protein selected from the group consisting of p53, JunD, JunB, c-Jun, v-Jun and Fas or a fusion protein comprising said JNK protein or fragment thereof and the activation domain of a transcription factor; the protein binding partner is an AP-1 family protein selected from the group consisting of p53, JunD, JunB, c-Jun, v-Jun, Fas or a fragment of said AP-1 family protein sufficient to bind to said JNK protein or said fragment, or a fusion protein comprising said AP-1 family protein or said fragment of said AP-1 family protein fused to a DNA binding domain; and the other protein is a protein selected from the group consisting of ATF-2, Elk1, CREB, NP-kappaB, and a WOX protein, or a fragment of said ATF-2, Elk1, CREB, NF-kappaB or WOX protein sufficient to bind JNK, or a fusion protein comprising said ATF-2, Elk1, CREB, NF-kappaB or WOX protein or said fragment fused to a DNA binding domain. In an alternative embodiment, the protein of interest is a JNK protein or fragment thereof sufficient to bind to an AP-1 family protein selected from the group consisting of p53, JunD, JunB, c-Jun, v-Jun and Fas or a fusion protein comprising said JNK protein or fragment thereof and the activation domain of a transcription factor; the protein binding partner is a protein selected from the group consisting of ATF-2, Elk1, CREB, NF-kappaB, and a WOX protein, or a fragment of said ATF-2, Elk1, CREB, NF-kappaB or WOX protein sufficient to bind JNK, or a fusion protein comprising said ATF-2, Elk1, CREB, NF-kappaB or WOX protein or said fragment fused to a DNA, binding domain; and the other protein is an AP-1 family protein selected from the group consisting of p53, JunD, JunB, c-Jun, v-Jun, Fas or a fragment of said AP-1 family protein sufficient to bind to said JNK protein or said fragment, or a fusion protein comprising said AP-1 family protein or said fragment of said AP-1 family protein fused to a DNA binding domain.

In a particularly preferred embodiment, the protein of interest comprises JNK (SEQ ID NO: 1) or a fragment thereof sufficient to bind to bind to one or more proteins selected from the group consisting of c-Jun (SEQ ID NO: 2), JIP2 (SEQ ID NO: 3), TI-JIP (SEQ ID NO: 4), JunD (SEQ ID NO: 5), JunB (SEQ ID NO: 6), ATF-2 (SEQ ID NO: 7), CREB2 (SEQ ID NO: 8), Elk1 (SEQ ID. NO: 9), NF-kappaB (SEQ ID NO: 10), human WOX3 (SEQ ID NO: 17), human WOX1 (SEQ ID NO: 18) and murine WOX1 (SEQ ID NO: 19) or a fusion protein comprising said JNK protein or fragment thereof and the activation domain of a transcription factor; and the binding partner protein and/or other protein is c-Jun (SEQ ID NO: 2), JIP2 (SEQ ID NO: 3), TI-JIP (SEQ ID NO: 4), JunD, (SEQ ID NO: 5), JunB (SEQ ID NO: 6), ATF-2 (SEQ ID NO: 7), CREB2 (SEQ ID NO: 8), Elk1 (SEQ ID NO: 9) or NF-kappaB (SEQ ID NO: 10), human WOX3 (SEQ ID NO: 17), human WOX1 (SEQ ID NO: 18) and murine WOX1 (SEQ ID NO: 19) or a fragment of c-Jun (SEQ ID NO: 2) or JIP2 (SEQ ID NO: 3) or TI-JIP (SEQ ID NO: 4) or JunD (SEQ ID NO: 5) or JunB (SEQ ID NO: 6) or ATF-2 (SEQ ID NO: 7) or CREB2 (SEQ ID NO: 8) or Elk1 (SEQ ID NO: 9) or NF-kappaB (SEQ ID NO: 10) or human WOX3 (SEQ ID NO: 17) or human WOX1 (SEQ ID NO: 18) or murine WOX1 (SEQ ID NO: 19) sufficient to bind to JNK (SEQ ID NO: 1), or a fusion protein comprising said c-Jun (SEQ ID NO: 2), JIP2 (SEQ ID NO: 3), TI-JIP (SEQ ID NO: 4), JunD (SEQ ID NO: 5), JunB (SEQ ID NO: 6), ATF-2 (SEQ ID NO: 7), CREB2 (SEQ ID NO: 8), Elk1 (SEQ ID NO: 9), NF-kappaB (SEQ ID NO: 10), human WOX3 (SEQ ID NO: 17), human WOX1 (SEQ ID NO: 18) and murine WOX1 (SEQ ID NO: 19) or said fragment fused to a DNA binding domain.

As exemplified herein, the mutated form of the protein of interest can be a mutated form of a JNK protein (SEQ ID NO: 1) wherein one or more amino acids of SEQ ID NO: 1 selected from the group consisting of E126, E129, L131, K300, R309, I310, D313, E314, Q317, P319, Y320 and W324 is substituted for another amino acid. Preferably, a mutated form of a JNK protein (SEQ ID NO: 1) comprises one or more mutations selected from the group consisting of L131R, R309W and Y320H. Even more preferably, a mutated form of a JNK protein (SEQ ID NO: 1) carries an amino acid substitution of one or more amino acids of SEQ ID NO: 1 selected from the group consisting of E126, E129, L131, K300, R309, I310, D313, E314, Q317, P319, Y320 and W324 for another amino acid; and the binding partner protein is a fusion protein comprising said TI-JIP (SEQ ID NO: 4) fused to a DNA binding domain.

In accordance with the preceding embodiments, it is particularly preferred that the DNA binding, domain is a GAL4 DNA binding domain or LexA operator binding domain or cI DNA binding domain. The DNA binding domains fused to the binding partner protein and protein of interest or fragment(s) thereof can be different, or the same.

In accordance with the preceding embodiments, it is particularly preferred that the activation domain fused to the protein of interest or a fragment thereof is selected from the group consisting of GAL4 activation domain, VP16 activation domain, mouse NF κB activation domain and B42 activation domain.

The method supra can be modified such that it includes the additional step of expressing a native form of the protein of interest and the native form of the binding partner protein and native forms of one or more other proteins that bind to the protein of interest such that the binding of the native form of the protein of interest to the native form of the binding partner protein to each other protein operably and separately controls the expression of a different reporter gene, and determining expression of each reporter gene. In accordance with this embodiment, a different level of expression of a reporter gene operably under the control of the binding between the native and mutated forms of the protein of interest and the native form of the binding partner protein and about the same level of expression of the other reporter genes indicates that the mutation in the mutated form of the protein of interest is within a region of the protein of interest that mediates the ability of the protein to bind to the binding partner protein.

The method supra can be modified such that it includes the additional step of producing a mutated from of the protein of interest. For example, one or more mutations can be introduced to a nucleotide sequence encoding the protein of interest or a fragment thereof such that the encoded peptide varies by one or more amino acids compared to nucleic acid encoding the native form of the protein of interest. The mutagenesis process can be selected from the group consisting of mutagenic PCR, replicating the nucleic acid in a bacterial cell that induces an accumulation of a random mutations through defects in DNA repair, site directed mutagenesis, and replicating the nucleic acid in a host cell exposed to a mutagenic agent. Mutagenic PCR is performed by a process selected from the group consisting of: (i) performing the PCR reaction in the presence of manganese; and (ii) performing the PCR in the presence of a concentration of dNTPs sufficient to result in misincorporation of nucleotides.

In a further embodiment, the present invention provides a method for identifying a region in a protein of interest that mediates the ability of the protein of interest to bind to a protein binding partner in a protein complex that comprises the protein of interest and the protein binding partner and one or more other proteins, said method comprising the steps of:
(i) providing a cell that comprises: (a) a nucleic acid comprising a counter-selectable reporter gene encoding a polypeptide that is capable of reducing cell growth or viability by providing a target for a cytotoxic or cytostatic compound or by converting a substrate to a cytotoxic or cytostatic product, said gene being positioned, downstream of a promoter comprising a cis-acting element such that expression of said gene is operably under the control of said promoter and wherein a fusion protein comprising the protein binding partner binds to said cis-acting element; (b) nucleic acid comprising a reporter gene other than the counter-selectable reporter gene of (a) positioned downstream of a promoter comprising the cis-acting element other than the cis-acting element at (a) such that expression of said reporter gene is operably under the control of said promoter and wherein a fusion protein comprising the other protein binds to said cis-acting element; (c) nucleic acid encoding a fusion protein comprising a variant or mutated form of the protein of interest and an activation domain that, activates expression of reporter genes (a) and (b); (d) nucleic acid encoding encoding a fusion protein that comprises the protein binding partner fused to a DNA binding domain of a transcription factor that binds to the cis-acting element in the counter selectable reporter gene (a) such that when the protein binding partner binds to the variant or mutated form of the protein of interest expression of the counter-selectable reporter gene at (a) is enhanced; and (e) nucleic acid encoding a fusion protein that comprises the other protein fused to a DNA binding domain of a transcription factor that binds to the cis-acting element in the reporter gene (b) such that when the other protein binds to the variant or mutated form of the protein of interest expression of the reporter gene at (b) is enhanced;
(ii) culturing said cell for a time and under conditions sufficient for the reporter genes at (i)(a) and (i)(b) and the fusion proteins at (i)(c), (i)(d) and (i)(e) to be expressed and for a native form of the protein of interest to bind to the protein binding partner and, to the other protein;
(iii) culturing the cell in the presence of the substrate or the cytotoxic or cytostatic compound such that the expressed counter-selectable reporter gene reduces the growth or viability of the cell unless said expression is inhibited or reduced by virtue of the variant or mutated form of the protein of interest having reduced binding to the protein binding partner;
(iv) culturing the cell under conditions sufficient to detect expression of the reporter gene at (i)(b) by virtue of an interaction between the variant or mutated form of the protein of interest and the other protein;
(v) detecting expression of the reporter genes at (i)(a) and (i)(b); and
(vi) selecting or screening for a cell that expresses the reporter gene at (i)(b) and has reduced or inhibited expression of the reporter gene at (i)(a) compared to a cell that expresses the native form of the protein of interest, wherein the selected cell carries a mutation in a region in the protein of interest that mediates the ability of the protein of interest to bind to the protein binding partner.

The step of providing a cell may comprise introducing nucleic acid into a cell that encodes at least one protein selected from the group consisting of the, protein of interest, the protein binding partner, and the other protein. Alternatively, or in addition, nucleic acid that comprises a reporter gene downstream of a promoter that comprises a cis-acting element to which the protein of interest, the protein binding partner, the other protein binds can be introduced to a cell. Alternatively, or in addition. nucleic acid that comprises a reporter gene downstream of a promoter that comprises a cis-acting element to which a fusion protein comprising the protein of interest, a fusion protein comprising the protein binding partner, or a fusion protein comprising the other protein binds can be introduced to a cell.

The skilled artisan is aware that the selection of a promoter for driving expression;.of the proteins will depend in part at least upon the choice of cell being used for the assay. The present invention is not to be limited to any specific cell type or by any specific selection of promoters, because a myriad of such expression systems are known to the skilled artisan. In one embodiment, the cell is a yeast cell, such as a yeast cell having a genotype selected from the group consisting of:
(i) MATa, ura3, trp1, met15, his3, his5, cyh2^{r}, lexAop-URA3, lexaop-CYH2, ade2;
(ii) MATa, his3, trp1, ura3, 6 LexA-LEU2, lys2::3 cIop-LYS2, CYH2^{R}, ade2::G418-pZero-ade2, met15::Zeo-pBLUE-met15;
(iii) MATa, his3, trp1, ura3, met15::pDR10, 6 LexA-LEU2, lys2::3 cIop-LYS2, CYH2^{r}, ade2::G418-pZero-ADE2; and
(iv) MATa; his3, trp1, ura3, met15::pDR10, 6 LexA-LEU2, lys2::3 cIop-LYS2, CYH2^{R}, ade2::G418-pZero-ADE2.

A suitable promoter for driving expression in a yeast cell can be selected from the group consisting of ADH1 promoter, GAL1 promoter, GAL4 promoter, CUP1 promoter, PHO4 promoter, PHO5 promoter, nmt promoter, RPR1 promoter and TEF1 promoter. In another embodiment, the cell is a nematode cell. A suitable promoter for driving expression in a nematode cell can be selected from the group consisting of osm-10, unc-54 and myo-2. In another embodiment, the cell is a fish cell. A suitable promoter for driving expression in a fish cell can be selected from the group consisting of zebrafish OMP promoter, GAP43 promoter and serotonin-N-acetyl transferase gene regulatory region. In another embodiment, the cell is a bacterial cell. A suitable promoter for driving expression in a bacterial cell can be selected from the group consisting of lacz promoter, Ipp promoter, temperature-sensitive λ_{L} promoter, temperature-sensitive λ_{R} promoter, T7 promoter, T3 promoter, SP6 promoter, tac promoter and lacUV5 promoter. In another embodiment, the cell is an insect cell. A suitable promoter for driving expression in an insect cell can be selected from the group consisting of OPEI2 promoter, actin promoter, dsh promoter and metallothionein promoter. In another embodiment, the cell is a plant cell. A suitable promoter for driving expression in a plant cell can be selected from the group consisting of amylase gene promoter, cauliflower mosaic virus 35S promoter, nopaline synthase (NOS) gene promoter, P1 promoter and P2 promoter. In another embodiment, the cell is a mammalian cell. A suitable promoter for driving expression in a mammalian cell can be selected from the, group consisting of a retroviral long terminal repeat (LTR), SV40 early promoter, SV40 late promoter, cytomegalovirus (CMV) promoter, CMV IE (cytomegalovirus immediate early) promoter, EF_{1α} promoter, EM7 promoter and UbC promoter.

Preferably, expression of the protein of interest or the protein binding partner is operably under the control of an inducible promoter sequence such that the level of expression of that protein is capable of being modulated in the cell. Preferred inducible promoters are copper inducible promoters (e.g., CUP) promoter), galactose-inducible promoters (e.g., GAL1 promoter), and phosphate-regulatable promoters (e.g., PHO4, PHO5). Preferably, the inducible promoter is the GAL1, PHO5 or CUP1 promoter, and the level of the counter-selectable reporter is modulated by varying the galactose, phosphate or copper concentration, respectively, of the medium in which the cell is cultured.

The counter-selectable reporter gene can also be operably connected to an inducible promoter such that the level of expression of said counter-selectable reporter gene is capable of being modulated in the cell. In accordance with this embodiment, the reporter genes can bind different proteins via different cis-acting elements, or alternatively, the cis-acting elements can be the same. Preferred cis-acting elements for docking the binding partner protein and the other protein are selected from a LexA operator, cI, and GAL4 recognition sequences. For example, each cis-acting element can bind to one or more DNA binding domains selected from the group consisting of a LexA DNA binding protein domain, cI protein domain and GAL4 protein domain, wherein said DNA binding domain is present in a fusion protein comprising the binding partner protein and/or the other protein.

A particularly preferred example of the present invention provides the following combination of reagents: (i) the reporter gene operably under the control of the interaction between the protein of interest and the protein binding partner is a counter selectable reporter gene selected from the group consisting of URA3, CYH2 and LYS2, or a gene encoding green fluorescent protein (GFP); and (ii) the reporter gene operably the control of the interaction between the protein of interest and the other protein is selected from the group consisting of LYS2 and cobA.

Preferably, the reporter gene operably under the control of the interaction between the protein of interest and the protein binding partner is URA3; and the reporter gene operably the control of the interaction between the protein of interest and the other protein is LYS2. Thus, cells are cultured separately in the presence of 5-FOA and α-AA and cells that do not survive selection on 5-FOA but survive on α-AA are selected.

Alternatively, the reporter gene operably under the control of the interaction between the protein of interest and the protein binding partner is CYH2; and the reporter gene operably the control of the interaction between the protein of interest and the other protein is LYS2. Thus, cells are cultured separately in the presence of cycloheximide and α-AA and cells that do not survive selection on cycloheximide but survive on α-AA are selected.

Alternatively, the reporter gene operably under the control of the interaction between the protein of interest and, the protein binding partner is LYS2; and the reporter gene operably the control of the interaction between the protein of interest and the other protein is cobA. In this case, fluorescent cells are cultured in the presence of α-AA and cells that do not survive selection on α-AA are selected. Naturally, to select such cells, replica plates or other cultures must be established to recover the cultured cells.

Alternatively, the reporter gene operably under the control of the interaction between the protein of interest and the protein binding partner is URA3; and the reporter gene operably the control of the interaction between the protein of interest and the other protein is cobA. In this case, fluorescent cells are cultured in the presence of 5-FOA and cells that do not survive selection on 5-FOA are selected. Naturally, to select such cells, replica plates or other cultures must be established to recover the cultured cells.

Alternatively, the reporter gene operably under the control of the interaction between the protein of interest and the protein binding partner is CYH2; and the reporter gene operably the control of the interaction between the protein of interest and the other protein is cobA. In this case, fluorescent cells are cultured in the presence of α-AA and cells that do not survive selection on cycloheximide are selected. Naturally, to select such cells, replica plates or other cultures must be established to recover the cultured cells.

Alternatively, the reporter gene operably under the control of the interaction between the protein of interest and the protein binding partner encodes GFP; and the reporter gene operably the control of the interaction between the protein of interest and the other protein is cobA. In this case, cells expressing only the cobA gene product are selected.

Alternatively, the reporter gene operably under the control of the interaction between the protein of interest and the protein binding partner is cobA; and the reporter gene operably the control of the interaction between the protein of interest and the other protein encodes GFP. In this case, cells expressing only GFP are selected.

As will be known to the skilled artisan, the nucleic acids encoding a fusion protein may be inserted into an expression vector to facilitate their maintenance and expression. Accordingly, the present invention clearly encompasses the additional process of introducing nucleic acid encoding one or more fusion proteins into an expression vector. Particularly preferred expression vectors are selected from the group consisting of pDEATH-Trp, (SEQ ID NO: 10), pJFK (SEQ ID NO: 11), pDD (SEQ ID NO: 12), pRT2 (SEQ ID NO: 13), pGMS19 (SEQ. ID NO: 15) and pDR10 (SEQ ID NO: 16). Alternatively, the vector pGILDA can be used. Other expression vectors are not to be excluded.

A second aspect of the present invention provides a method for determining an inhibitor of an interaction between a protein of interest and a protein binding partner in a cell, said method comprising:
(i) expressing a mutated form of the protein of interest and the native form of the binding partner protein and native forms of one or more other proteins that bind to the protein of interest such that the binding of the mutated form of the protein of interest to the native form of the binding partner protein and each other protein operably controls the expression of a different reporter gene, and selecting or screening for modified expression of the reporter gene that is operably under the control of a binding between the protein of interest and the binding partner protein and unmodified expression of each other reporter gene, wherein said modified expression indicates that the mutation is within a region in the protein of interest that mediates the ability of the protein to bind to the binding partner protein;
(ii) determining a fragment of the mutated form of the protein of interest said fragment comprising the region that mediates the ability of the protein to bind to the binding partner protein; and
(iii) determining a fragment in the native form of the protein of interest that is functionally equivalent, to (ii) wherein said fragment inhibits the interaction between the native form of the protein of interest and the binding partner.

Preferably, (i) comprises performing the method according to any embodiment supra to thereby identify a mutation within a region in a protein of interest that mediates the ability of the protein to bind to a binding partner protein.

Preferably, the process of the invention comprises recovering a fragment in the native form of the protein of interest having an amino acid sequence that encompasses all or part of the mutated site in the mutated form of the protein of interest.

Preferably, a fragment in the native form of the protein of interest having an amino acid sequence that encompasses all or part of the mutated site in the mutated form of the protein of interest is synthesized e.g., as a peptide of no more than about 50 amino acid residues in length.

A third aspect of the present invention provides a process for determining or validating a protein interaction as a therapeutic drug target or validation reagent comprising:
(i) performing the process according to any embodiment supra thereby determining a fragment in a protein of interest that inhibits the interaction between the protein of interest and a binding partner protein; and
(ii) expressing the fragment in a cell or organism as a dominant negative inhibitor and determining a phenotype of the cell or organism that is modulated by the target protein or target nucleic acid wherein a modified phenotype of the cell or organism indicates that the protein interaction is a therapeutic target or validation reagent.

A fourth aspect of the present invention provides a process for determining or validating a protein interaction as a therapeutic drug target or validation reagent comprising:
(i) performing the method according to any embodiment supra to thereby identify a, mutation within a region in a protein of interest that mediates the ability of a protein of interest to bind to a binding partner protein; and
(ii) expressing nucleic acid encoding the mutated form of the protein of interest in a model organism to thereby produce a knock-in of the mutant allele; and
(iii) detecting the phenotype of that mutant wherein a modified phenotype of the cell or organism indicates that the protein interaction is a therapeutic target or validation reagent.

Preferably the process for identifying a therapeutic or, prophylactic compound comprises:
(i) performing the process according to embodiment supra to thereby determine a fragment in a protein of interest that inhibits the interaction between the protein of interest and a binding partner protein; and
(ii) identifying a compound having the inhibitory activity of the fragment e.g., a mimetic compound of the inhibitory peptide.

Preferably, the process further comprises:
(a) optionally, determining the structure of the compound or modulator identified in a screen for mimetic activity with the inhibitory peptide; and
(b) providing the compound or modulator or the name or structure of the compound or modulator such as, for example, in a paper form, machine-readable form, or computer-readable form.

Preferably, the process of the invention further comprises producing or synthesizing the compound.

A further aspect of the present invention provides a method for determining or validating a protein interaction as a therapeutic drug target or validation reagent comprising:
(a) expressing a mutated form of a protein of interest and the native form of a binding partner protein and native forms of one or more other proteins that bind to the protein of interest such that the binding of the mutated form of the protein of interest to the native form of the binding partner protein and each other protein operably controls the expression of a different reporter gene, and selecting or screening for modified expression of the reporter gene that is operably under the control of a binding between the protein of interest and the binding partner protein and unmodified expression of each other reporter gene, wherein said modified expression indicates that the mutation is within a region in the protein of interest that mediates the ability of the protein to bind to the binding partner protein;
(b) determining a fragment of the mutated form of the protein of interest said fragment comprising the region that mediates the ability of the protein to bind to the binding partner protein;
(c) determining a fragment in the native form of the protein of interest that is functionally equivalent to (b) wherein said fragment inhibits the interaction between the native form of the protein of interest and the binding partner; and
(d) expressing the fragment at (c) in a cell or organism as a dominant negative inhibitor and determining a phenotype of the cell or organism that is modulated by the target protein or target nucleic acid wherein a modified phenotype of the cell or organism indicates that the protein interaction is a therapeutic target or validation reagent.

In an alternative embodiment, rather than expressing a fragment in a cell or organism, the corresponding mutant form of the gene encoding the native form of a native protein of interest is expressed in a model organism (eg; a ‘knock-in’ of the mutant allele made by homologous recombination and detecting the phenotype of that mutant.

A further aspect of the present invention provides a method for identifying a therapeutic or prophylactic compound comprising:
(a) expressing a mutated form of a protein of interest and the native form of a binding partner protein and native forms of one or more other proteins that bind to the protein of interest such that the binding of the mutated form of the protein of interest to the native form of the binding partner protein and each other protein operably controls the expression of a different reporter gene, and selecting for modified expression of the reporter gene that is operably under the control of a binding between the protein of interest and the binding partner protein and unmodified expression of each other reporter gene, wherein said modified expression indicates that the mutation is within a region in the protein of interest that mediates the ability of the protein to bind to the binding partner protein;
(b) determining a fragment of the mutated form of the protein of interest said fragment comprising the region that mediates the ability of the protein to bind to the binding partner protein;
(c) determining a fragment in the native form of the protein of interest that is functionally equivalent to (b) wherein said fragment inhibits the interaction between the native form of the protein of interest and the binding partner; and
(d) identifying a mimetic compound of the fragment at (c).

A further aspect of the present invention provides a method for identifying a therapeutic or prophylactic compound comprising:
(a) expressing a mutated form of a protein of interest and the native form of a binding partner protein and native forms of one or more other proteins that bind to the protein of interest such that the binding of the mutated form of the protein of interest to the native form of the binding partner protein and each other protein operably controls the expression of a different reporter gene, and selecting for modified expression of the reporter gene that is operably under the control of a binding between the protein of interest and the binding partner protein and unmodified expression of each other reporter gene, wherein said modified expression indicates that the mutation is within a region in the protein of interest that mediates the ability of the protein to bind to the binding partner protein;
(b) determining a critical fragment (or specific residues therein) of the mutated form of the protein of interest said fragment comprising the region that mediates the ability of the protein to bind to the binding partner protein;
(c) modelling the structure of the region of the protein of interest which contains the critical fragment-(or specific residues therein); and
(d) designing a small molecule inhibitor which binds to the fragment (or specific residues therein) in the native form of the protein of interest wherein said small molecule inhibitor inhibits the interaction between the native form of the protein of interest and the binding partner.

A further aspect of the present invention provides a method for identifying a an allosteric therapeutic or prophylactic inhibitor compound comprising:
(a) expressing a mutated form of a protein of interest and the native form of a binding partner protein and native forms of one or more other proteins that bind to the protein of interest such that the binding of the mutated form of the protein of interest to the native form of the binding partner protein and each other protein operably controls the expression of a different reporter gene, and selecting for modified expression of the reporter gene that is operably under the control of a binding between the protein of interest and the binding partner protein and similarly altered expression of each other reporter gene, wherein said modified expression indicates that the mutation is within a region in the protein of interest that mediates the ability of the protein to bind to both the binding partner protein and the other protein;
(b) determining by means of Western Blotting that the mutation does not cause the protein of interest to be unstable or truncated (by for example the introduction of a non-sense mutation).
(c) determining a critical fragment (or specific residues therein) of the mutated form of the protein of interest said fragment comprising the region that mediates the ability of the protein to bind to the binding partner protein;
(d) modelling the structure of the region of the protein of interest which contains the critical fragment (or specific residues therein); and
(e) designing a small molecule inhibitor which binds to the fragment (or specific residues therein) in the native form of the protein of interest, wherein said small molecule inhibitor inhibits the interaction between the native form of the protein of interest and the binding partner.

A further aspect of the present invention provides an isolated peptide comprising an amino acid sequence that inhibits the interaction between a protein of interest and a protein binding partner in a cell when determined by a method comprising:
(a) expressing a mutated form of the protein of interest and the native form of the binding partner protein and native forms of one or more other proteins that bind to the protein of interest such that the binding of the mutated form of the protein of interest to the native form of the binding partner protein and each other protein operably controls the expression of a different reporter gene, and selecting for modified expression of the reporter gene that is operably under the control of a binding between the protein of interest and the binding partner protein and unmodified expression of each other reporter gene, wherein said modified expression indicates that the mutation is within a region in the protein of interest that mediates the ability of the protein to bind to the binding partner protein;
(b) determining a fragment of the mutated form of the protein of interest said fragment comprising the region that mediates the ability of the protein to bind to the binding partner protein; and
(c) determining a fragment in the native form of the protein of interest that is functionally equivalent to (b) wherein said fragment inhibits the interaction between the native form of the protein of interest and the binding partner.

### BRIEF DESCRIPTION OF THE DRAWINGS

FIG. 1 is a schematic representation of the MAPK signalling pathways involving p38, Extracellular Receptor Kinases (ERKs) and c-Jun N-terminal kinases (JNKs) in mammalian cells during stress, injury or hemorrhagic shock, including ischemia.

FIG. 2 is a graphical representation showing the effect of cell-permeable peptide inhibitor of the interaction between JNK1 (SEQ ID NO: 1) and c-Jun (SEQ ID NO: 2), designated Truncated Inhibitor of JNK based on JIP (SEQ ID NO: 3), herein referred to as “TI-JIP” (SEQ ID NO: 4) on neurons. Neurons were either maintained under normal conditions (control) or subjected to oxygen-glucose deprivation in the absence of TI-JIP peptide (OGD) or in the presence of 2 μM TI-JIP for different times (TI-JIP and TI-JIP 1 h). Data show that TI-JIP protects neurons from simulated stroke in the form of oxygen-glucose deprivation.

FIG. 3 is a schematic representation showing changes to amino acid residues in JNK that disrupt binding of the protein to TI-JIP peptide, in particular Leu169 (L169), Arg 347 (R347) and Tyr358 (Y358). The ATP binding site is also indicated.

FIG. 4 is a schematic representation of the pDEATH-Trp vector (SEQ ID NO: 11). The pDEATH-Trp vector comprises a minimal ADH promoter for constitutive expression in yeast cells; a T7 promoter for expression of a nucleic acid fragment in bacterial, cells; a nucleic acid encoding a SV-40 nuclear localization signal to force any expressed polypeptide into the nucleus of a yeast cell; a CYC1 terminator, for termination of transcription in yeast cells; a nucleic acid encoding a peptide conferring ampicillin resistance, for selection in bacterial cells; a nucleic acid encoding TRP1 which allows auxotrophic yeast to grow in media lacking tryptophan; a pUC origin of replication, to allow the plasmid to replicate in bacterial cells; and a 2μ origin of replication, to allow the plasmid to replicate in yeast cells.

FIG. 5 is a schematic representation of the pJFK vector (SEQ ID NO: 12). The pJFK vector comprises a GAL1 promoter for inducible expression in yeast cells; a nuclear localization signal to force any expressed polypeptide into the nucleus of a yeast cell; a nucleic acid encoding an activation domain derived from the B42 protein, to be expressed as a fusion with a polypeptide of interest in a “N”-hybrid screen; an ADH terminator or termination of transcription in yeast cells; a 2μ origin of replication, to allow the plasmid to replicate in yeast cells; an HIS5 gene to allow auxotrophic yeast to grow in media lacking histidine; a nucleic acid encoding a peptide conferring ampicillin resistance, for selection in bacterial cells; and a nucleic acid encoding a peptide conferring kanamycin resistance.

FIG. 6 is a schematic representation of the pDD vector (SEQ ID NO: 13). The pDD vector comprises a GAL1 promoter for inducible expression in yeast cells; a nucleic acid encoding a LEXA protein, to be expressed as a fusion with a polypeptide of interest in a “n”-hybrid screen; an ADH terminator or termination of transcription in yeast cells; a 2μ origin of replication, to allow the plasmid to replicate in yeast cells; an -HIS5 gene to allow auxotrophic, yeast to grow in media lacking histidine; a nucleic acid encoding a peptide conferring ampicillin resistance, for selection in bacterial cells; and a nucleic acid encoding a peptide conferring kanamycin resistance.

FIG. 7 is a schematic representation of the vector pRT2 (SEQ ID NO: 14) containing the following features:

a first fluorescent reporter gene cassette comprising the gfp gene encoding green fluorescent protein placed operably under control of a chimeric yeast operable LexA/GAL1 promoter having 8 LexA operator sites, and upstream of the yeast ADH1 terminator;

a second fluorescent reporter gene cassette comprising the cobA gene encoding a fluorescent protein placed operably under control of a chimeric cI/GAL1 promoter having 3 cI operator sites;

a wild-type yeast operable selectable marker gene (ADE2) for conferring adenine auxotrophy oh cells expressing said gene;

a selectable marker gene for conferring resistance to the antibiotic kanamycin in bacteria;

a bacterial origin of replication (colE1); and

a eukaryotic origin of replication (2 Ori).

FIG. 8 is a schematic representation of the pGMS19 vector (SEQ ID NO: 15). The pGMS19 vector comprises a GAL1 promoter for inducible expression in yeast cells; a nucleic acid encoding a cI protein, to be expressed as a fusion with a polypeptide of interest in a “n”-hybrid screen; an ADH terminator or termination of transcription in yeast cells; a CEN/ARS origin of replication, to allow the plasmid to replicate in yeast cells; an MET15 gene to allow auxotrophic yeast to grow in media lacking methionine; and a nucleic acid encoding a peptide conferring kanamycin resistance. The pGMS19 vector is of particular use in a dual-bait two-hybrid systems in combination with a LexA fused bait protein.

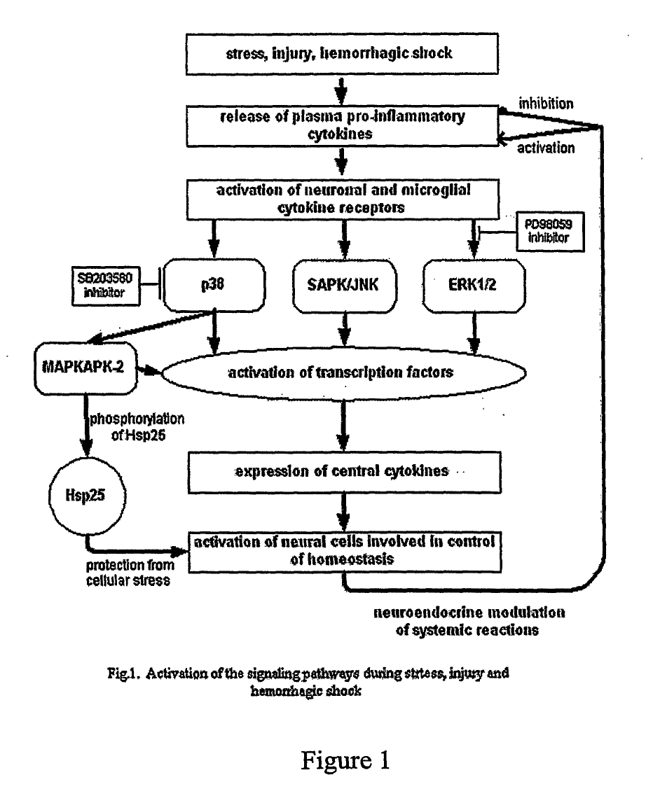

FIG. 9 is a schematic of reverse two-hybrid screening principles and the optimized conditions for screening a JNK mutant library. FIG. 1a shows that when TI-JIP and the wild-type JNK fusion protein (AD-JNK) interact, the URA3 reporter gene was expressed to convert 5′fluoroorotic acid (5′FOA) in the yeast medium into a toxic product, thereby resulting in cell death. In FIG. 9b, TI-JIP was screened against a library of random JNK mutants (AD-JNK(MUT)), such that those cells in which mutant JNK proteins interacted with TI-JIP died, and those cells expressing mutant JNK proteins which lost the ability to interact with TI-JIP survived because the URA3 reporter gene was not transcribed and 5′FOA was not converted into a toxic product. In FIG. 9c, cells survived by virtue of the fact that no JNK protein was present and the activation domain alone could not interact with TI-JIP. Illustrated are the optimised screening conditions that permitted maximal death of the positive control yeast (TI-JIP and AD-JNK) with minimal death of negative control yeast (TI-JIP and AD). The upper panels show yeast growth in the presence of Galactose (0.08% Gal), Raffinose (2% Raft) and a low, concentration of Glucose (0.05% Gluc), which induced bait and prey expression. The lower panels show yeast growth in the presence of Glucose (2% Gluc), which repressed bait and prey expression and was indicative of the total number of yeast plated on the medium.

FIG. 10 is a photographic representation showing colonies expressing full-length AD-JNK fusion proteins. FIG. 10a shows typical results of PCR screening to detect the presence of JNK1 DNA in yeast that survived reverse two-hybrid screening. This distinguished colonies expressing pJG4-5-JNK1 plasmids from colonies expressing the empty pJG4-5 prey vector, which resulted in background survival in the screen. FIG. 10b shows the results of Western blotting using HA antibody to detect the HA-tagged, AD-JNK1 fusion protein (58 kDa) (solid arrow) in yeast that had been shown to express a pJG4-5-JNK1 plasmid by PCR screening. The number of yeast that expressed a full length AD-JNK1 fusion protein was found to be relatively low. The bracketed region indicates the presence of truncation mutations of JNK1, which were detected in some samples.

FIG. 11a is a graphical representation showing mutation data from reverse two-hybrid screening, indicating the mutations identified in the 16 mutant JNK sequences. Mutations were calculated per region of JNK secondary structure and then normalized for the length of the secondary structure. Two regions were identified with 50% hits/length (#1 and #2), and point mutations were designed to address the importance of these regions (Leu-110-His and Val-219-Asp, respectively).

FIG. 11b is a diagrammatic representation showing four views of the JNK protein (i-iv) to illustrate all faces of the three-dimensional structure, with the positions of mutated amino acids shown in black JNK mutants containing 5 or less mutations per JNK sequence. Limitation of mutations to this level per molecule reduces background interference. This resulted in 27 identified amino acid mutations (Lys-Glu, Gln-102-Arg, Leu-110-His, Leu-110-Pro, Met-121-Lys, Asp-124-Tyr, Leu-131-Arg, Leu-131-Phe, Net-135-Lys, Lys-140-Glu, Lys-166-Glu, Tyr-190-His, Asn-205-Asp, Cys-213-Ser, Val-219-Asp, Glu-261-Lys, Asn-262-Ser, Leu-279-Pro, Asn-287-Tyr, Ser-292-Cys, Arg-309-Trp, Asp-313-Gly, Tyr-320-His, Asp-339-Tyr, Trp-352-Arg, Met-361-Val, Glu-365-Val). Note that Leu-110 and Leu-131 were mutated on two separate occasions. The positions of these mutated amino acids in JNK1 were mapped onto the crystal structure of the JNK3 protein.

FIG. 12a is a diagrammatic representation showing four views of the JNK protein (i-iv) to illustrate all faces of the three-dimensional structure, with the positions of single point mutations indicated and positions of mutated amino acids shown in black. Single point mutants define important residues on JNK for its interaction with TI-JIP. Point mutants of JNK were constructed by site-directed mutagenesis to assess the relative contribution of different hot-spots to the JNK-TI-JIP interaction. Amino acids located in putative mutational hot-spots were targeted for further investigation.

FIG. 12b is a representation of β-galactosidase overlay assay results (left) showing the ability of JNK mutants to interact with TI-JIP and Western blot assay data to detect the HA-tagged full length JNK1 mutant proteins (right). Of the nine point mutations tested, three point mutations (Leu-131-Arg, Arg-309-Trp, Tyr-320-His) rendered JNK incapable of interaction with TI-JIP. Western blotting was performed to ensure that the lack of interaction did not arise from problems associated with protein expression. Two independent colonies were tested for each mutation to confirm the results of the overlay assay and Western blotting.

FIG. 13 is a diagrammatic representation of a space filling model of JNK1 protein showing the location of JNK1 residues Leu-131 and Tyr-320 relative to other residues implicated in MAPK docking interactions. (i), Ribbon structure of JNK1 for comparison with space-filling models. (ii), Space-filling structure of JNK with Leu-131 and Tyr-320 highlighted in black, which were shown in this study to be critical for the interaction between JNK1 and the TI-JIP inhibitor, based on the KIM of JIP-1. (iii), As per (ii), with CD residues Asp-326, Glu-329 and Tyr-130, and SD site residues Ser-161 and Asp-162 highlighted in black. (iv), As per (ii), with JNK1 residues 107-131 and 159-165 highlighted in black, which correspond to residues in the related p38 MAPK that were thought to mediate hydrophobic contacts with KIM sequences present in interacting partners. As per (ii), with residues Glu-329 and Glu-331 highlighted in black, which were shown to be critical for the interaction between JNK2 and JIP-1.

FIG. 14a is a photographic representation showing expression of wild-type (WT) JNK and mutants (n=2) in transfected COS cells. The wild type JNK construct was pCMV-FLAG-JNK1. Equivalent constructs with point mutations corresponding to JNK1(Leu-131-Arg), JNK1(Arg-309-Trp) and JNK1(Tyr-320-His) were also used.

FIG. 14b is a representation showing a typical autoradiograph (upper panel) illustrating phosphorylation of GST-c-Jun(1-135) by wild-type (WT) JNK and mutants (n=2) for COS cells transfected as described in the legend to FIG. 14a. Transfected cells were incubated without sorbitol or exposed to hyperosmotic shock (0.5 M sorbitol, 30 min) prior to lysis. FLAG-tagged JNK1 and mutants were immunoprecipitated from cell lysates and then assayed for activity towards GST-c-Jun(1-135) using in vitro kinase assays. Coomassie Blue staining (lower panel) confirmed substrate loading.

FIG. 14c is a photographic representation showing a typical autoradiograph (upper panel) illustrating phosphorylation of GST-c-Jun(1-135) by wild-type (WT) JNK and mutants (n=2) for COS cells transfected as described in the legend to FIG. 14a, or co-transfected with a constitutively-active MEKK1 construct (CA-MEKK1). Cells were lysed, and FLAG-tagged JNK and mutant proteins were immunoprecipitated from cell lysates. Immunoprecipitates were subjected to in vitro kinase assays using a GST-c-Jun(1-135) substrate. Coomassie Blue staining (lower panel) confirmed substrate loading.

FIG. 15a is a representation showing that JNK mutants were not activated by constitutively-active MKK4 (MKK4(ED)) or MKK7. COS cells were transfected with pCMV-FLAG-JNK1, or equivalent constructs with point mutations corresponding to JNK1(Leu-131-Arg), JNK1(Arg-309-Trp) and JNK1(Tyr-320-His). JNK proteins were immunoprecipitated from transfected cell lysates, and immunoprecipitates were used as the substrates in in vitro kinase assays with GST-MKK4(ED). Following separation by SDS-PAGE, activation of JNK and mutant proteins was assessed by autoradiography (upper panel) (n=2). Coomassie Blue staining (lower panel) confirmed substrate loading.

FIG. 15b is a representation showing that JNK mutants were not activated by constitutively-active MKK4 (MKK4(ED)) or MKK7. COS cells were transfected with either, JNK or mutant constructs alone, or co-transfected with pEBG-MKK7β1. Lysates were separated by SDS-PAGE and then transferred to nitrocellulose. Immunoblotting was performed using an antibody directed towards the dual-phosphorylated activated form of JNK to detect the amount of JNK activation stimulated by co-expressed MKK7 (upper panel) (n=2). Total JNK protein expression was assessed using antibodies directed against JNK1 and the FLAG epitope tag. The Tyr-320-His mutant consistently had a reduced SDS-PAGE mobility relative to wild-type JNK1, despite sequencing the construct to confirm its identity.

### DETAILED DESCRIPTION OF THE PREFERRED EMBODIMENTS

### 1. Identification of the Interaction Interface of a Protein

One aspect of the present invention provides a method for identifying the interaction interface between two protein binding partners. In one embodiment there is provided a method for identifying a region in a protein of interest that mediates the ability of the protein to bind to a binding partner protein in a protein complex that comprises more than two proteins, said method comprising expressing a mutated form of the protein of interest and the “native form of the binding partner protein and native forms of one or more other proteins that bind to the protein of interest such that the binding of the mutated form of the protein of interest to the native form of the binding partner protein and each other protein operably controls the expression of a different reporter gene, and selecting for modified expression of the reporter gene that is operably under the control of a binding between the protein of interest and the binding partner protein and unmodified expression of each other has reporter gene, wherein said modified expression indicates that the mutation is within a region in the protein of interest that mediates the ability of the protein to bind to the binding partner protein.

By “interaction interface” is meant the portion or region of one protein that is in close physical proximity or relation with another in a protein complex, such as, for example, a protein complex having a function in vivo. As will be known to those skilled in the art, an interaction interface will comprise one or more amino acid residues in one of the protein binding partners that are essential for such binding or interaction to occur and/or that mediate binding of one protein to another protein. The amino acid residues in the interaction interface may be contiguous or non-contiguous with respect to the primary structure (i.e., the amino acid sequence) of the protein.

Those skilled in the art will be aware that an interaction interface is useful in its isolated form as a dominant negative mutant to inhibit a protein-protein interaction. Accordingly, notwithstanding that an interaction interface may consist of a single amino acid residue, the term “interaction interface” shall be taken for practical purposes to encompass any peptides consisting of at least 5 contiguous amino acid residues in length derived from the amino acid sequence of a protein wherein said contiguous amino acid residues comprise one or more amino acid residues in the protein that are essential for binding of that protein to another protein, or mediate an interaction between that protein and another protein. Thus, an interaction interface includes amino acid residues flanking an amino acid residue that is required for binding in the primary structure of a protein.

It is to be understood that the “interaction interface” of a protein will not extend to any peptides consisting of or comprising an amino acid sequence of a full-length protein. In fact, an interaction interface will generally have an upper length of about 50 amino acid residues that are contiguous with the primary sequence of a protein. In a preferred embodiment, the interaction interface of a protein will comprise an amino acid sequence consisting of about 5-10 amino acid residues that are contiguous with the primary sequence of a protein, or about 15-20 contiguous amino acid residues in length or about 20-25 contiguous amino acid residues in length or about 25-30 contiguous amino acid residues in length.

Those skilled in the art will also understand that the term “protein binding partner” means a protein that is involved in a close physical relation or association with another protein in a protein complex. As used throughout this specification and in the claims unless the context requires otherwise, the term “protein binding partner” shall be taken to mean a specific proteinaceous species, including peptides and polypeptides that is involved in a close physical relation or association with a specified protein of interest.

The term “protein of interest” as used herein shall be taken to mean a protein species in which one or more amino acid residues that are essential for binding to the “protein binding partner” are being determined, or are the subject of a claim.

Preferably, a direct interaction between the protein of interest and the protein binding partner, or a direct interaction between a fusion protein comprising the protein of interest and a fusion protein comprising the protein binding partner, is sufficient to bind to the upstream region (5′-UTR) of a reporter gene and activate its expression. Alternatively, there may also be one or more additional proteins in the assay that bind, to the protein binding partner or to the protein of interest, to produce a functional protein complex that is capable of binding to and activating expression of a reporter gene.

As used herein, the “other protein” shall be taken to mean a protein that binds to a protein of interest and optionally to a protein binding partner of the protein of interest, the only requirement being that the other protein does not inhibit the interaction between the protein of interest and the protein binding partner such that said interaction is abrogated. In one embodiment, the other protein(s) will bind to a different site in the protein of interest to the interaction site between the protein of interest and the protein binding partner.

The interaction between the other protein and the protein of interest may be direct or indirect. In one embodiment, an “adaptor” protein or peptide can be included in the assay to mediate or enhance the interaction. For example, the protein of interest may comprise a DNA binding domain fusion between the GAL4 DNA or LexA operator binding domain of a transcription factor and an amino acid sequence that dimerizes with the adaptor polypeptide, whilst the other protein comprises an activation domain fusion between a transcriptional activator domain, such as the GAL4 activator domain, and an amino acid sequence that dimerizes with the adaptor protein. Alternatively, there may be direct interaction between the protein of interest and the other protein, without a requirement for an adaptor protein to facilitate their dimerization.

Moreover, because the “other protein” is included as an internal control for the correct conformation of the protein of interest, it is not necessary for the “other protein” to be a protein that forms part of a naturally occurring protein complex with both the protein of interest and the protein binding partner. For example, the protein of interest may interact with the protein binding partner under a specified environmental condition or at a particular stage of development that is different to the environmental/developmental milieu in which the protein of interest binds to the other protein(s). In this case, the method of the present invention will require an artificial combination in vitro of distinct protein complexes that occur in vivo. In an alternative embodiment, the protein of interest may interact with the protein binding partner in vivo under a specified environmental condition or at a particular stage of development that is the same as the environmental/developmental milieu in which the protein of interest binds to the other protein(s). In this case, the method of the present invention may require an artificial combination in vitro of distinct protein complexes that occur in vivo, or alternatively, rely upon the reconstitution in vitro of a protein complex that is known to occur in vivo.

In another preferred alternative embodiment, the ‘protein partner’ and the ‘other protein’ may represent two allelic or mutant forms of the same protein or even two orthologues of the protein encoded by the genomes of distinct species.

Fragments of a protein of interest, fragments of a protein binding partner, and fragments of the other protein(s) that retain the ability of the full-length protein to bind to another protein in the method of the present invention can also be used. Accordingly, the terms “protein of interest”, “protein binding partner” and “other protein” clearly encompass such functionally equivalent fragments. In fact, in many instances it is preferred to express such fragments, because gene, constructs for their expression are easier to produce than gene constructs expressing full-length proteins.

As used herein, the term “native form” with reference to a protein binding partner or other protein shall be taken to mean a full-length protein that has an amino, acid sequence corresponding to the sequence of a naturally-occurring isoform of the protein, or a fragment of the full-length protein.

It will be understood from the preceding description that the selection of a particular species of protein of interest, protein binding partner, and other protein, for use in the inventive method will vary according to the interaction interface being determined. In view of the general applicability of the present invention to determining any interaction interface, the only requirement being that the protein of interest is capable of binding to more than one protein or peptide, the present invention is not to be limited to particular species of proteins or peptides or a particular species of interaction.

Notwithstanding the preceding paragraph, several protein-protein interactions are described below for the purposes of exemplification of the invention. In one embodiment, the protein of interest is a MAP kinase protein, such as, for example, a stress-activated MAP kinase protein selected from the group consisting of a p38 protein, an SAPK protein, a JNK protein and an ERK protein.

The term “p38 protein” shall be taken to refer to a stress-activated serine/threonine protein kinase of mammals, such as, for example, a human, rat or mouse protein, belonging to the MAP kinase superfamily and having an estimated molecular mass of about 38 kDa. The term “p38” further encompasses proteins designated “CSBP” or “RK” or “p38 MAPK” or “SAPK-2” or an isoform of p38 selected from the group consisting of “p38-alpha”, “p38-beta”, “p38-gamma” and “p38-delta”. Those skilled in the art will readily be able to obtain and identify a p38 protein from the literature (see, eg., Cano and Mahadevan, Trends Biochem. Sci. 20, 117-122, 1995; Davis, Trends Biochem. Sci. 19, 470-473, 1994; Eyers et al., Chem and Biol 5, 321-328, 1995; Jiang et al, J Biol Chem 271, 17920-17926, 1996; Kumar et al, Biochem Biophys Res Comm 235, 533-538, 1997; Stein et al., J Biol Chem 272, 19509-19517, 1997; Li et al., Biochem Biophys Res Comm 228, 334-340, 1996; Wang et al., J Biol Chem 272, 23668-23674, 1997; Wang et al., J Biol Chem 273, 2161-2168, 1998; and the references cited therein). An exemplary human p38 amino acid sequence is provided by Han et al., Science 265, 808-811, 1994 or Lee et al., Nature 372, 739-746, 1994, and Bernd et al. U.S. al. U.S. Ser. No. 10/197,315 (Publication No. 20030059881) which are incorporated herein by reference. The term “p38” shall also be understood to encompass any variants of the sequences disclosed by Han et al., Science 265, 808-811, 1994 or Lee et al., Nature 372, 739-746, 1994, and Bernd et al. U.S. Ser. No. 10/197,315 (Publication No. 20030059881) which are functionally equivalent to a p38 protein as defined herein.

Diverse extracellular stimuli, including ultraviolet light, irradiation, heat shock, high osmotic stress, pro-inflammatory cytokines and certain mitogens, trigger a stress-regulated protein kinase cascade culminating in activation of p38 through phosphorylation on a TGY motif within the kinase activation loop (ie., residues Thr180 to Tyr182). The p38 protein appears to play a major role in apoptosis, cytokine production, transcriptional regulation, and cytoskeletal reorganization, and has been causally implicated in sepsis, ischemic heart disease, arthritis, human immunodeficiency virus infection, and Alzheimer's disease. The availability of specific inhibitors helps to clarify the role that p38 plays in these processes, and may ultimately offer therapeutic benefit for certain critically ill patients.

The terms “SAPK protein” or “JNK protein” shall be taken to refer to a stress-activated protein kinase of mammals, including but not limited to JNK1, JNK2, JNK3, an isoform of JNK1, JNK2 or JNK3 (Gutta et al., EMBO J., 1996, 15, 2760), or another member of the JNK family of proteins whether they function as Jun N-terminal kinases per se (that is, phosphorylate Jun at a specific amino terminally located position) or not. Preferred JNK proteins are capable of reversibly binding and phosphorylating the transcription factor cJun and/or the activator protein 1 (AP-1) transcription factor complex comprising c-Jun and/or c-Fos. SAPK/JNK effectively acts as a universal pivot point, with targets to both a ternary complex transcription factor (ELK-1) and activating transcription factor 2 (ATF-2). The ternary complex factor ELK-1, once activated by SAPK/JNK, leads to positive regulation of the c-Fos promoter resulting in increased expression of the c-Fos protein with concomitant increases in AP-1 levels. Targeting of ATF-2, which can form heterodimers with c-Jun, is another suitable route to initiate increases in AP-1 expression. Given the myriad of possibilities for activating AP-1, it is quite apparent that the SAPK/JNK is a model transduction junction for amplifying a given extracellular, signal. The SAPK/JNK proteins are encoded by at least three genes, and as with all MAPKs, each SAPK/JNK protein isoform contains a characteristic Thr-X-Tyr phospho-acceptor loop domain, where X indicates any amino acid structurally suitable for a loop domain.

An exemplary SAPK/JNK protein is described by Derijard et al Cell 76 (6), 1025-1037, 1994 which is incorporated herein by way of reference. For the purposes of nomenclature, the amino acid sequence of this JNK protein is set forth herein as SEQ ID NO: 1. Preferred JNK proteins will comprise an amino acid sequence that is at least about 70% identical to the sequence set forth in SEQ ID NO: 1.

The term “extracellular regulated protein kinase” or “MAP2 kinase” or “ERK” shall be taken to refer to a stress-activated protein kinase of mammals, including but not limited to a protein selected from the group consisting of ERK1, ERK2, ERK3, ERK4, an isoform of ERK1, ERK2, ERK3 or ERK4, or another member of the ERK/MAP-2 kinase family of proteins whether they function as MAP-2 kinases per se (that is, phosphorylate MAP-2) or not MAP-2 kinases or ERKs are generally expressed in the central nervous system, and comprise a phospho-acceptor sequence of Thr-Glu-Tyr, an amino-terminal kinase domain followed by an extensive carboxy-terminal tail of unknown function that comprises several proline-rich motifs indicative of binding sites with SH3 domains. The SH3 adaptor proteins are instrumental in linking the initial activation of the kinase to the downstream components of any signal transduction pathway. Although the stimuli that recruit ERK have not been well identified, environmental stresses such as osmotic shock and oxidant stress have been shown to substantially activate ERK and similar substrates.

The amino acid sequences of several ERK proteins are described by Boulton et al U.S. Ser. No. 6,297,035 and U.S. Ser. No. 6,303,358, which are incorporated herein by reference:

In accordance with this embodiment the protein binding partner and other protein(s) are proteins that bind to the MAP kinase protein, such as for example, a protein substrate of the MAP kinase. Such proteins will be known to those skilled in the art. Preferred protein binding partners and other proteins are selected from the group consisting of: These transcription factors include c-Jun (SEQ ID NO: 2), JIP2 (SEQ ID NO: 3), JunD (SEQ ID NO: 5), JunB (SEQ ID NO: 6), ATF-2 (SEQ ID NO: 7), CREB2 (SEQ ID NO: 8), Elk1 (SEQ ID NO: 9), NF-kappaB (SEQ ID NO: 10), human WOX3 (SEQ ID NO: 17), human WOX1 (SEQ ID NO: 18) and murine WOX1 (SEQ ID NO: 19). Other AP1 family proteins, such as, for example, v-Jun or Fas can also be used. MKK3 (Davis et al., U.S. Ser. No. 6,541,605), MKK4/SEK1 (Davis et al., U.S. Ser. No. 6,541,605), MKK7, a Bcl-2 family protein (eg., BIM), cdc47, and S6 kinase protein are also useful.

In a preferred embodiment, the method of the present invention is applied to the identification of an interaction interface in a JNK protein. In accordance with this embodiment, the protein of interest is a JNK protein, and the protein binding partner is a protein selected from the group consisting of an AP-1 family protein (eg p53, JunD, JunB, c-Jun, v-Jun, or Fas), and a fragment of an AP-1 family protein that interacts with JNK, and the other protein is a protein selected from the group consisting of ATF-2, Elk1, CREB, NF-kappaB and a WOX protein, a fragment of ATF-2 that interacts with JNK, a fragment of Elk1 that interacts with JNK, a fragment of CREB that interacts with JNK, a fragment of NF-kappaB that interacts with JNK and a fragment of a WOX protein that interacts with JNK.

Alternatively, wherein the protein of interest is a JNK protein, and the protein binding partner is a protein selected from the group consisting of ATF-2, Elk1, CREB; NF-kappaB, a fragment of ATF-2 that interacts with JNK, a fragment of Elk1 that interacts with JNK, a fragment of CREB that interacts with JNK, a fragment of NF-kappaB that interacts with JNK, a fragment of WOX1 that interacts with JNK and a fragment of WOX3 that interacts with JNK, and the other protein is a protein selected from the group consisting of an AP-1 family protein (eg p53, JunD, JunB, c-Jun, v-Jun, or Fas), and a fragment of an AP-1 family protein that interacts with JNK.

Other combinations of proteins for identifying the interaction site(s) of JNK are not to be excluded.

In an alternative embodiment, the protein of interest is the oncoprotein SCL or a dimerization region of SCL, and, the protein binding partner and other protein are selected from the group consisting of: LMO1, LMO2, DRG, mSin3A, E47, a dimerization region of LMO1, a dimerization region of LMO2, a dimerization region of DRG, a dimerization region of mSin3A, and a dimerization region of E47.

Preferably, the protein of interest, protein binding partner and other protein are presented in the inventive method as a fusion protein with the DNA binding domain (DBD) of a transcription factor or a transcription activator domain (AD). In accordance with this embodiment, those skilled in the art of hybrid screening approaches will be aware that two proteins that interact with each other are generally expressed separately as a fusion with a DBD and an AD. Similarly, in the present context, it is preferred that the protein of interest is expressed as a fusion protein with an AD and the protein binding partner and other protein are each expressed as fusion proteins with a different DBD to avoid inappropriate docking on the wrong reporter gene.

When the appropriate association between proteins occurs, a functional transcription factor is reconstituted, and expression of a reporter gene placed under the control of the reconstituted transcription factor occurs.

Preferred DNA binding domains include, for example, the GAL4 DNA binding domain or LexA DNA binding protein which binds to the lexA operator.

Preferred activation domains include, for example, the GAL4 activation domain, the VP16 activation domain, the mouse NF κB activation domain and fortuitous activation domains such as the B42 activation domain encoded by the E. coli genome.

Preferably, but not necessarily, each interaction will utilize a different DNA binding domain.

For example, fusion proteins may be constructed between an oncoprotein and a DNA binding domain and/or a DNA activation domain. For example, a sequence of nucleotides encoding or complementary to a sequence of nucleotides encoding $CL may be fused to a transcriptional activation domain and a nucleotide sequence encoding LMO1 may be fused to the LexA DNA binding domain while the E47 protein may be fused to the the CI DNA binding domain.

Alternatively, wherein the protein of interest is a transcription factor with an endogenous transcriptional activation domain, such as, for example, the Fos transcription factor that binds to JUN, expression of that protein as a fusion protein with a DNA binding domain or an activation domain may not be required, provided that the protein fused to an appropriate domain to enable it to bind to the upstream region of a promoter to which a reporter gene is linked and provided that the protein is able to activate expression of the reporter gene in the host organism of the screen such as yeast.

Mutated Form of a Protein of Interest

In a preferred embodiment, the present invention further comprises the step, of producing a mutated from of the protein of interest.

As used herein, the term “mutated form” with reference to a protein species shall be taken to mean a variant of the protein that comprises one or more amino acid substitutions, deletions or additions relative to the amino acid sequence of the native polypeptide. By “native polypeptide” is meant a form of a polypeptide that is functional in binding to a native form of a protein binding partner.

Those skilled in the art will be aware of several methods for producing a mutated form of a protein.

In one embodiment, the nucleotide sequence encoding the protein of interest is mutated by a process such that the encoded peptide, varies by one or more amino acids compared to the “template”-nucleic acid fragment. The “template” may have the same nucleotide sequence as the original nucleic acid fragment in its native context (ie. in the gene from which it was derived). Alternatively, the template may itself be an intermediate variant that differs from the original nucleic acid fragment as a consequence of mutagenesis. Mutations include at least one nucleotide difference compared to the sequence of the original fragment. This nucleic acid change may result in for example, a different amino acid in the encoded peptide, or the introduction or deletion of a stop codon. Mutations that introduce amino acid substitutions are preferred, however not essential to the present invention, because the screening process selects against or nonsense mutations.

In one embodiment, nucleic acid encoding the protein of interest or a fragment thereof is modified by a process of mutagenesis selected from the group consisting of, mutagenic PCR, replicating the nucleic acid in a bacterial cell that induces an accumulation of a random mutations through defects in DNA repair, by site directed mutagenesis, of by replicating the nucleic acid in a host cell exposed to a mutagenic agent such as for example radiation, bromo-deoxy-uridine (BrdU), ethylnitrosurea (ENU), ethylmethanesulfonate (EMS) hydroxylamine, or trimethyl phosphate. Alternatively, the nucleic acid can be exposed to the the mutagenic agent in vitro, prior to transformation.

In a preferred embodiment, the nucleic acid is modified by amplifying a nucleic acid fragment using mutagenic PCR. Such methods is include a process selected from the group consisting of: (i) performing the PCR reaction in the presence of manganese; and (ii) performing the PCR in the presence of a concentration of dNTPs sufficient to result in misincorporation of nucleotides.

Methods of inducing random mutations using PCR are well known in the art and are described, for example, in Dieffenbach (ed) and Dveksler (ed) (In: PCR Primer: A Laboratory Manual, Cold Spring Harbour Laboratories, NY, 1995). Furthermore, commercially available kits for use in mutagenic PCR are obtainable, such as, for example, the Diversify PCR Random Mutagenesis Kit (Clontech) or the GeneMorph Random Mutagenesis Kit (Stratagene).

In one embodiment, PCR reactions are performed in the presence of at least about 200 μM manganese or a salt thereof, more preferably at least about 300 μM manganese or a salt thereof, or even more preferably at least about 500 μM or at least about 600 μM manganese or a salt thereof. Such concentrations manganese ion or a manganese salt induce from about 2 mutations per 1000 base pairs (bp) to about 10 mutations every 1000 bp of amplified nucleic acid (Leung et al Technique 1, 11-15, 1989).

In another embodiment, PCR reactions are performed in the presence of an elevated or increased or high concentration of dGTP. It is preferred that the concentration of dGTP is at least about 25 μM, or more preferably between about 50 μM and about 100 μM. Even more preferably the concentration of dGTP is between about 100 μM and about 150 μM, and still more preferably between about 150 μM and about 200 μM. Such high concentrations of dGTP result in the misincorporation of nucleotides into PCR products at a rate of between about 1 nucleotide and about 3 nucleotides every 1000 bp of amplified nucleic acid (Shafkhani et al BioTechniques 23, 304-306, 1997).

PCR-based mutagenesis is preferred for the mutation of the nucleic acid fragments of the present invention, as increased mutation rates is achieved by performing additional rounds of PCR.

In another preferred embodiment, the nucleic acid encoding the protein of interest is mutated by inserting said nucleic acid into a host cell that is capable of mutating nucleic acid. Such host cells are deficient in one or more enzymes, such as, for example, one or more recombination or DNA repair enzymes, thereby enhancing the rate of mutation to a rate that is rate approximately 5,000 to 10,000 times higher than for non-mutant cells. Strains particularly useful for the mutation of nucleic acids carry alleles that modify or inactivate components of the mismatch repair pathway. Examples of such alleles include-. alleles selected from the group consisting of mutY, mutM, mutD, mutt, mutA, mutC and mutS. Bacterial cells that carry alleles that modify or inactivate components of the mismatch repair pathway are well known in the art, such as, for example the XL-1Red, XL-mutS and XL-mutS-Kan^{r} bacterial cells (Stratagene).

Alternatively the nucleic acid is cloned into a nucleic acid vector that is preferentially replicated in a bacterial cell by the repair polymerase, Pol I. By way of exemplification, a Pol I variant strain will induce a high level of mutations in the introduced nucleic acid vector. Such a method is described by Fabret et al (In: Nucl. Acid Res, 28, 1-5 2000), which is incorporated herein by reference.

In a further preferred embodiment, alanine scanning mutagenesis is carried out. Those skilled in the art will be aware that alanine scanning mutagenesis introduces substitutions of alanine residues in a protein for other amino acid residues. Commercially available methods and reagents are available for performing alanine scanning mutagenesis of nucleic acid encoding the protein of interest, such as, for example, by cloning said nucleic acid into a suitable expression vector e.g., pcDNA3.1 (Stratagene) and using the resulting recombinant vector with the Quickchange Mutagenesis kit supplied by Stratagene.

Preferably, mutagenesis is performed under conditions such that the coding region of the nucleic acid encoding the protein of interest is saturated with mutations across the mutant library, however each molecule that is mutated comprises only a single or a few mutations. Preferably, the, mutated nucleic acid should encode a variant or mutated form of the protein of interest that differs from the native form by less than about 5 amino acid substitutions and more preferably only 1 or 2 amino acid substitutions. Accordingly, a library of mutants is produced wherein the aligned sequences of the encoded proteins have mutations spanning the entire protein sequence.

Each mutant form of the protein of interest is then separately expressed with the native form of the protein binding partner and other protein. This is achieved, for example, by transformation of suitable host cells expressing the protein binding partner and other protein and containing nucleic” acid comprising each reporter gene with the library of mutants under conditions such that a single mutant sequence is introduced to each transformant.

Reporter Genes

As used herein, the term “reporter gene” shall be taken to mean a genomic gene, cDNA or other nucleic acid encoding a protein that is physically measurable or detectable, wherein the level of expression of the protein can be measured and/or correlated with a change in the binding activity between the protein of interest and the protein binding partner or between the protein of interest and the other protein(s). Reporter genes are well known in the art, and include, but are not limited to, nucleic acids encoding proteins that fluoresce, for example the red fluorescent protein (i.e, cobA gene product) or green fluorescence protein (i.e., the gfp gene product), nucleic acids encoding proteins that induce a colour change in the presence of a substrate, for example E coli β-galactosidase or LacZ or GusA, and nucleic acids encoding proteins that confer growth characteristics on a cell by (for example) complementing auxotrophic mutations (such as for example the HIS3 gene). Genes that confer resistance to an antibiotic (eg., ampicillin, kanamycin, G418, tetracycline, neomycin, etc), or other toxic chemical compound are also useful in this context.

Counter selectable reporter genes encode a lethal product when expressed in a cell, or alternatively, encode a protein or enzyme that converts a non-toxic substrate to a toxic product. Counter selectable reporter genes suitable for this purposes include, for example, the yeast URA3, structural gene which is lethal to yeast cells when expressed in the presence of 5-fluororotic acid. (5-FOA); the yeast CYH2 gene which is lethal when expressed in the presence of the drug cycloheximide; and the yeast LYS2 gene which is lethal in the presence of the drug αα-aminoadipate (α-AA). Those skilled in the art will be aware that reverse n-hybrid screens routinely employ such counter selectable reporter genes. (e.g, WO 99/35282).

The only requirement for a suitable reporter gene is the capability of being expressed in a manner that is readily detected, such as by the phenotype said expression confers on the cell (for, example, restoration of prototrophy for a particular nutrient by complementation, or conditional lethality in the presence of a particular substrate), or alternatively, by expressing an enzyme activity, or a protein detectable by immunoassay or colorimetric detection, or fluorescence.

Suitable reporter genes include those encoding Escherichia coli β-galactosidase enzyme, the firefly luciferase protein (Ow et al, Science 234:856-859, 1986; Thompson et al, Gene 103:171-177, 1991) the green fluorescent protein (Prasher et al, Gene 111:229-233, 1992; Chalfie et al, Science 263:802-805, 1994; Inouye and Tsuji, FEBS Letts 341:277-280, 1994; Cormack et al, Gene, 1996; Haas et al, Curr. Biol. 6:315-324, 1996; see also GenBank Accession No. U55762); and the red fluorescent proteins of Discosoma (Matz et al, Nature Biotechnology 17: 969-973, 1999) or Propionibacterium freudenreichii, (Wildt and Deuschle, Nature Biotechnology 17: 1175-1178, 1999). Additionally, the HIS3 gene (Larson et al. EMBO J. 15 (5):1021, 1996; Condorelli et al., Cancer Research 56:5113, 1996; Hsu et al., Mol. Cell. Biol. 11:3037, 1991; Osada et al., Proc. Natl. Acad. Sci. USA 92:9585, 1995) and LEU2 gene (Mahajan et al., Oncogene 12:2343, 1996), the GUSA and LYS2 genes (are also useful.

It will be apparent from the preceding description that each interaction in the inventive method (i.e., the interaction between the protein of interest and the protein binding partner, and each additional interaction between the protein of interest and each other protein), operably regulates the expression of a different reporter gene. The selection of suitable reporter genes will largely influence the manner in which the selection of modified expression of the reporter gene that is operably under the control of a binding between the protein of interest and the binding partner protein and modified or unmodified expression of each other reporter gene is performed.

In a preferred embodiment, the reporter gene that is operably under the control of the interaction between the protein of interest and the protein binding partner is a counter selectable reporter gene, preferably a counter selectable reporter gene selected from the group consisting of URA3, CYH2 and LYS2. In accordance with this embodiment, modified expression of the reporter gene is carried out under conditions such that cells expressing the reporter gene do not survive selection on 5-FOA (in the case of URA3), or cycloheximide (in the case of CYH2) or α-AA (in the case of LYS2). Also in accordance with this embodiment, the reporter gene(s) placed operably the control of the interaction(s) between the protein of interest and the other protein(s) will be a reporter gene other, than the aforementioned counter selectable reporter gene, since those interactions are to be maintained.

It will be apparent to those skilled in the art that a reporter gene other than a counter selectable reporter gene can also be used for detecting the interaction between the protein of interest and the protein binding partner, since reduced expression of a reporter gene when the interaction is abrogated is generally detectable using such systems.

In a particularly preferred embodiment, the reporter gene/s operably under the control of the interaction between the protein of interest and the protein binding partner is at least one a counter selectable reporter gene selected from the group consisting of URA3, CYH2 and LYS2, or a gene encoding a fluorescent protein such as GFP, and the reporter gene(s) placed operably the control of the interaction(s) between the protein of interest and the other protein(s) is selected from the group consisting of LYS2 and cobA. In accordance with this embodiment, modified expression of the reporter gene is carried out under conditions such that cells expressing the reporter gene do not survive selection on 5-FOA (in the case of URA3), or cycloheximide (in the case of CYH2) or α-AA (in the case of LYS2), however cells in which the interaction between the protein of interest and the other protein(s) is maintained are selected by their ability to fluoresce at an appropriate wavelength (in the case, of fluorescent reporters) or grow in media lacking a certain nutrient such as lysine or leucine.

Combinations of a counter selectable reporter gene with one or more genes that encode fluorescent proteins are particularly preferred for high throughput applications, where large numbers of samples are screened in batches. By virtue of the phenotype that counter selectable reporter genes produce on a cell, they are particularly preferred for rapidly eliminating background in which the interaction between the protein of interest and the protein binding partner is not abrogated. Additionally, fluorescence generated from fluorescent proteins is readily assayed by fluorometry or fluorescence activated cell sorting (FACS), a technique known to those skilled in the art.

The expression of multiple reporter genes can also be placed operably under the control of the interaction between the protein of, interest and the protein binding partner, to reduce background effects and the selection of “false positives” in the screening process. Preferably, such multiple reporter genes will include at least one counter selectable reporter gene and at least one gene encoding a fluorescent protein.

Persons skilled in the art will be aware of how to utilize reporter genes in performing the invention described herein, without undue experimentation. For example, the coding sequence of the gene encoding such a reporter molecule may be modified for use in the cell line of interest (e.g. human cells, yeast cells) in accordance with known codon usage preferences. Additionally the translational efficiency of mRNA derived from non-eukaryotic sources may be improved by mutating the corresponding gene sequence or otherwise introducing to said gene sequence a Kozak consensus translation initiation site (Kozak, Nucleic Acids Res. 15: 8125-8148, 1987). Likewise the promoter sequences controlling expression from the reporter genes may be modified to minimise background expression and to put them more tightly under the control of factors binding to introduced exogenous elements such as lexA operators.

Expression of Proteins and Reporter Genes

Expression of the protein of interest, protein binding partner, other protein(s) and reporter genes, requires nucleic acid encoding each protein and nucleic acid comprising each reporter gene to be placed operably in connection with a promoter sequence.

Reference herein to a “promoter”. is to be taken in its broadest context and includes the transcriptional regulatory sequences of a classical genomic gene, including the TATA box which is required for accurate transcription initiation in eukaryotic cells, with or without a CCAAT box sequence and additional regulatory elements (i.e. upstream activating sequences, enhancers and silencers). Promoters may also be lacking a TATA box motif, however comprise one or more “initiator elements” or, as in the case of yeast-derived promoter sequences, comprise one or more “upstream activator sequences” or “UAS” elements. For expression in prokaryotic cells such as, for example, bacteria, the promoter should at least contain the −35 box and −10 box sequences.

A promoter is usually, positioned upstream or 5′ of a structural gene, the expression of which it regulates. Furthermore, the regulatory elements comprising a promoter are usually positioned within about 2 kb of the start site of transcription of the gene.

In the present context, the term “promoter” is also used to describe a synthetic or fusion molecule, or derivative that confers, activates or enhances expression of the subject reporter molecule in a cell.

Preferred promoters may contain additional copies of one or more specific regulatory elements, to further enhance expression of the gene and/or to alter the spatial expression and/or temporal expression. For example, regulatory elements which facilitate the enhanced expression of a gene by galactose or glucose or copper may be placed adjacent to a heterologous promoter sequence driving expression of the gene. Promoters comprising regulatory elements of the GALL or CUP1 promoters are particularly preferred for titration of the, expression of one or more proteins in response to galactose or copper, respectively, in the culture medium in which the host cell is grown.

Suitable promoters also include those from genes that are induced by the absence of a nutrient, for example the PHO5 gene is induced by a reduction in the amount of phosphate in the media in which a cell is cultured.

Placing a gene operably under the control of a promoter sequence means positioning the said gene such that its expression is controlled by the promoter sequence. Promoters are generally positioned 5′ (upstream) to the genes that they control. In the construction of heterologous promoter/structural gene combinations it is generally preferred to position the promoter at a distance from the gene transcription start site that is approximately the same as the distance between that promoter and the gene it controls in its natural setting, i.e., the gene from which the promoter is derived. As is known in the art, some variation in this distance can be accommodated without loss of promoter function. Similarly, the preferred positioning of a regulatory sequence element with respect to a heterologous gene to be placed under its control is defined by the positioning of the element in its natural setting, i.e., the genes from which it is derived. Again, as is known in the art, some variation in this distance can also occur.

Examples of promoters suitable for use in regulating expression of the protein of interest or the protein binding partner or the other protein include viral, fungal, yeast, insect, animal and plant promoters, especially those that can confer expression in a eukaryotic cell, such as, for example, a yeast cell or a mammalian cell.

Those skilled in the art will recognise that the choice of promoter will depend upon the nature of the cell being transformed and the molecule to be expressed. Such persons will be readily capable of determining functional combinations of minimum promoter sequences and operators for cell types in which the inventive method is performed.

Whilst the invention can be performed in yeast cells, the inventors clearly contemplate modifications wherein the invention is performed entirely in bacterial or mammalian cells or in non-cellular systems (e.g., ribosome display, mRNA display or covalent display), utilizing appropriate promoters that are operable therein to drive express ion of the various assay components under such conditions. Such embodiments are within the ken of those skilled in the art.

In a particularly preferred embodiment, the promoter is a yeast promoter, mammalian promoter, a bacterial or bacteriophage promoter, selected from the group consisting of: MYC, GAL1, CUP1, PGK1, ADH1, ADH2, PHO4, PHO5, HIS4, HIS5, TEF1, PRB1, TDH1, GUT1, SPO13, CMV, SV40, LAC, TEF, EM7, SV40, and T7 promoter sequences. Suitable yeast promoters are known to those skilled in the art and a re listed in standard manuals such as Guthrie and Fink (In: Guide to Yeast Genetics and Molecular and Cell Biology Academic Press, ISBN 01 21822540, 2002).

Typical promoters suitable for expression in viruses of bacterial cells and bacterial cells such as for example a bacterial cell selected from the group comprising E. coli, Staphylococcus sp, Corynebacterium sp., Salmonella sp., Bacillus sp., and Pseudomonas sp., include, but are not limited to, the lacz promoter, the Ipp promoter, temperature-sensitive λ_{L} or λ_{R} promoters, T7 promoter, T3 promoter, SP6 promoter or semi-artificial promoters such as the IPTG-inducible tac promoter or lacUV5 promoter. A number of other systems for obtaining expression in bacterial cells are well-known in the art and are described for example, in Ausubel et al (In: Current Protocols in Molecular Biology. Wiley Interscience, ISBN 047 150338, 1987), U.S. Pat. No. 5,763,239 (Diversa Corporation) and (Sambrook et al (In: Molecular Cloning: Molecular Cloning: A Laboratory Manual, Cold Spring Harbor Laboratories, New York, Third Edition 2001).

Typical promoters suitable for expression in yeast cells such as for example a yeast cell selected from the group comprising Pichia pastoris, S. cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Typical promoters suitable for expression in insect cells, or in insects, include, but are not limited to, the OPEI2 promoter, the insect actin promoter isolated from Bombyx muri, the Drosophila sp. dsh promoter (Marsh et al Hum. Mol. Genet. 9, 13-25, 2000) and the inducible metallothionein promoter. Preferred insect cells for expression of the recombinant polypeptides include an insect cell selected from the group comprising, BT1-TN-5B1-4 cells, and Spodoptera frugiperda cells (eg., sf19 cells, sf21 cells). Suitable insects for the expression of the nucleic acid fragments include but are not limited to Drosophila sp. The use of S. frugiperda is also contemplated.

Promoters for expressing peptides in plant cells are known in the art, and include, but are not limited to, the Hordeum vulgare amylase gene promoter, the cauliflower mosaic virus 35S promoter, the nopaline synthase (NOS) gene promoter, and the auxin inducible plant promoters P1 and P2.

Typical promoters suitable for expression in a virus of a mammalian cell, or in a mammalian cell, mammalian tissue or intact mammal include, for example a promoter selected from the group consisting of, retroviral LTR elements, the SV40 early promoter, the SV40 late promoter, the cytomegalovirus (CMV) promoter, the CMV IE (cytomegalovirus immediate early) promoter, the EF_{1α} promoter (from human elongation factor la), the EM7 promoter, the UbC promoter (from human ubiquitin C).

As will be known to the skilled artisan, the promoter can also be positioned in the expression vector or gene construct into which the prokaryote or eukaryote nucleic acid fragment is inserted.

In one embodiment, the proteins and reporter genes are expressed in vitro. According to this embodiment, a gene construct is produced that comprises a protein-encoding nucleic acid (“open reading frame” or “ORF”) and a promoter sequence and appropriate ribosome binding site which can both be present in the expression vector or added to said nucleic acid before it is inserted into the vector. Typical promoters for the in vitro expression include, but are not limited to the T3 or T7 (Hanes and Pluckthun Proc. Natl. Acad. Sci. USA, 94 4937-4942 1997) bacteriophage promoters.

In another embodiment, the gene construct optionally comprises a transcriptional termination site and/or a translational termination codon. Such sequences are well known in the, art, and is incorporated into oligonucleotides used to amplify the ORF of a reporter gene or an ORF encoding the protein of interest, protein binding partner, or other protein. Alternatively, a transcriptional termination site and/or a translational termination codon can be present in the expression vector or gene construct before the nucleic acid is inserted.

In another embodiment, the ORF is cloned into an expression vector. The term “expression vector” refers to a nucleic acid molecule that has the ability confer expression of nucleic acid to which it is operably connected, in a cell or in a cell free expression system.

Within the context of the present invention, it is to be understood that an expression vector may comprise a promoter as defined herein, a plasmid, bacteriophage, phagemid, cosmid, virus sub-genomic or genomic fragment, or other nucleic acid capable of maintaining and or replicating heterologous DNA in an expressible format. Many expression vectors are commercially available for expression in a variety of cells. Selection of appropriate vectors is within the knowledge of those having skill in the art.

Typical expression vectors for in vitro expression or cell-free expression have been described and include, but are not limited to the TNT T7 and TNT T3 systems (Promega), the pEXP1-DEST and pEXP2-DEST vectors (Invitrogen).

Numerous expression vectors for expression of recombinant polypeptides in bacterial cells and efficient ribosome binding sites have been described, such as for example, PKC30 (Shimatake and Rosenberg, Nature, 292, 128, 1981); pKK173-3 (Amann and Brosius, Gene 40, 183, 1985), pET-3 (Studier and Moffat, J. Mol. Biol. 189, 113, 1986); the pCR vector suite (Invitrogen), pGEM-T Easy vectors (Promega), the pL expression vector suite (Invitrogen) the pBAD/TOPO or pBAD/thio—TOPO series of vectors containing an arabinose-inducible promoter (Invitrogen, Carlsbad, Calif.), the latter of which is designed to also produce fusion proteins with a Trx loop for conformational constraint of the expressed protein; the pFLEX series of expression vectors (Pfizer nc., CT, USA); the pQE series of expression vectors (QIAGEN, CA, USA), or the pL series of expression vectors (Invitrogen), amongst others.

Expression vectors for expression in yeast cells are preferred and include, but are not limited to, the pACT vector (Clontech), the pDBleu-X vector, the pPIC vector suite (Invitrogen), the pGAPZ vector suite (Invitrogen), the pHYB vector (Invitrogen), the pYD1 vector (Invitrogen), and the pNMT1, pNMT41, pNMT81 TOPO vectors (Invitrogen), the pPC86-Y vector (Invitrogen), the pRH series of vectors (Invitrogen), pYESTrp series of vectors (Invitrogen). Particularly preferred vectors are the pACT vector, pDBleu-X vector, the pHYB vector, pJG4-5, pGilda, pEG202, the pPC86 vector, the pRH vector and the pYES vectors, which are all of use in various ‘n’-hybrid assays described herein. Furthermore, the pYD1 vector is particularly useful in yeast display experiments in S. cerevesiae. A number of other gene construct systems for expressing the nucleic acid fragment of the invention in yeast cells are well-known in the art and are described for example, in Giga-Hama and Kumagai (In: Foreign Gene Expression in Fission Yeast: Schizosaccharomyces Pombe, Springer Verlag, ISBN 3540632700, 1997) and Guthrie and Fink (In: Guide to Yeast Genetics and Molecular and Cell Biology Academic Press, ISBN 0121822540, 2002).

A variety of suitable expression vectors, containing suitable promoters and regulatory sequences for expression in insect cells are well known in the art, and include, but are not limited to the pAC5 vector, the pDS47 vector, the pMT vector suite (Invitrogen) and the pIB vector suite (Invitrogen).

Furthermore, expression vector's comprising promoters and regulatory sequences for expression of polypeptides in plant cells are also well known in the art and include, for example, a promoter selected from the group, pSS, pB1121 (Clontech), pZ01502, and pPCV701 (Kuncz et al, Proc. Natl. Acad. Sci. USA, 84 131-135, 1987).

Expression vectors that contain suitable promoter sequences for expression in mammalian cells or mammals include, but are not limited to, the pcDNA vector suite supplied by Invitrogen, the pCI vector suite (Promega), the pCMV vector suite (Clontech), the pM vector (Clontech), the pSI vector (Promega), the VP16 vector (Clontech) and the pDISPLAY vectors (Invitrogen). The pDISPLAY vectors are of particular use in mammalian display studies with the expressed nucleic acid fragment targeted to the cell surface with, the Igκ leader sequence, and bound to the membrane of the cell through fusion to the PDGFR transmembrane domain. The pM and VP16 vectors are of particular use in mammalian two-hybrid studies.

In a particularly preferred embodiment, the expression vector is selected from the group consisting of pDEATH-Trp, (SEQ ID NO: 10), pJFK (SEQ ID NO: 11), pDD (SEQ ID NO: 12), pRT2 (SEQ ID NO: 13), pGMS19 (SEQ ID NO: 15) and pDR10 (SEQ ID NO: 16). These vectors are described in more detail in the figure legends.

Alternatively, or in addition the pGILDA vector described in WO99/35282 can also be used.

Methods of cloning DNA into nucleic acid vectors for expression of encoded polypeptides are well known in the art and are described for example in, Ausubel et al (In: Current Protocols in Molecular Biology. Wiley Interscience, ISBN 047 150338, 1987) or Sambrook et al (In: Molecular Cloning: Molecular Cloning: A Laboratory Manual, Cold Spring Harbor Laboratories, New York, Third Edition 2001).

It is preferred that when the gene constructs are to be introduced to and/or maintained and/or propagated and/or expressed in bacterial cells, either during generation of said gene constructs, or screening of said gene constructs, that the gene constructs contain an origin of replication that is operable at least in a bacterial cell. A particularly preferred origin of replication is the ColE1 origin of replication. A number, of gene construct systems containing origins of replication are well-known in the art and are described for example, in Ausubel et al (In: Current Protocols in Molecular Biology. Wiley Interscience, ISBN 047 150338, 1987), U.S. Pat. No. 5,763,239 (Diversa Corporation) and (Sambrook et al (In: Molecular Cloning: Molecular Cloning: A Laboratory Manual, Cold Spring Harbor Laboratories, New York, Third Edition 2001).

It is also preferred that when the gene constructs are to be introduced to and/or maintained and/or propagated and/or expressed in yeast cells, either during generation of said gene constructs, or screening of said gene constructs, that the gene constructs contain an origin of replication that is operable at least in a yeast cell. One preferred origin of replication is the CEN/ARS4 origin of replication. Another particularly preferred origin of replication is the 2-micron origin of replication. A number of gene construct systems containing origins of replication are well-known in the art and are described for example, in Ausubel et al (In: Current Protocols in Molecular Biology. Wiley Interscience, ISBN 047 150338, 1987) and (Sambrook et al (In: Molecular Cloning: Molecular Cloning: A Laboratory Manual, Cold Spring Harbor Laboratories, New York, Third Edition 2001).

Gene constructs will preferably comprise a selectable marker. As used herein the term “selectable marker” shall be taken to mean a protein or peptide that confers a phenotype on a cell expressing said selectable marker that is not shown by those cells that do not carry said selectable marker. Examples of selectable markers include, but are not limited to the dhfr resistance gene, which confers resistance to methotrexate (Wigler, et al., 1980, Natl. Acad. Sci. USA 77:3567; O'Hare, et al., 1981, Proc. Natl. Acad. Sci. USA 78:1527); the gpt resistance gene, which confers resistance to mycophenolic acid (Mulligan & Berg, 1981, Proc. Natl. Acad. Sci. USA 78:2072); the neomycin phosphotransferase gene, which confers resistance to the aminoglycoside G-418 (Colberre-Garapin, et al., 1981, J. Mol. Biol. 150:1); and the hygromycin resistance gene (Santerre, et al., 1984, Gene 30:147). Alternatively, marker genes is catalyse reactions resulting in a visible outcome (for example the production of a blue color when β galactosidase is expressed in the presence of the substrate molecule 5-bromo-4-chloro-3-indoyl-β-D-galactoside) or confer the ability to synthesise particular amino acids (for example the HIS3 gene confers the ability to synthesize histidine).

Recombinant gene constructs capable of expressing the protein of interest, protein binding partner, other protein or reporter gene product are introduced to and preferably expressed within a cellular host or organism. Methods of introducing the gene constructs into a cell or organism for expression are well known to those skilled in the art and are described for example, in Ausubel et al (In: Current Protocols in Molecular Biology. Wiley Interscience, ISBN 047 150338, 1987), U.S. Pat. No. 5,763,239 (Diversa Corporation) and (Sambrook et al (In: Molecular Cloning: Molecular Cloning: A Laboratory Manual, Cold Spring Harbor Laboratories, New York, Third Edition 2001). The method chosen to introduce the gene construct in depends upon the cell type in which the gene construct is to be expressed.

In one embodiment, the cellular host is a bacterial cell. Means for introducing recombinant DNA into bacterial cells include, but are not limited to electroporation or chemical transformation into cells previously treated to allow for said transformation.

In another embodiment, the cellular host is a yeast cell. Means for introducing recombinant DNA into yeast cells include a method chosen from the group consisting of electroporation, and PEG mediated transformation.

In another embodiment, the cellular host is a plant cell. Means for introducing recombinant DNA into plant cells include a method selected from the group consisting of Agrobacterium mediated transformation, electroporation of protoplasts, PEG mediated transformation of protoplasts, particle mediated bombardment of plant tissues, and microinjection of plant cells or protoplasts.

In yet another embodiment, the cellular host is an insect cell. Means for introducing recombinant DNA into plant cells include a method chosen from the group consisting of, infection with baculovirus and transfection mediated with liposomes such as by using cellfectin (Invitrogen).

In yet another embodiment, the cellular host is a mammalian cell. Means for introducing recombinant DNA into mammalian cells include a means selected from the group comprising microinjection, transfection mediated by DEAE-dextran, transfection mediated by calcium phosphate, transfection mediated by liposomes such as by using Lipofectamine (Invitrogen) and/or cellfectin (Invitrogen), PEG mediated DNA uptake, electroporation, transduction by Adenoviuses, Adeno-associated viruses, Papilloma viruses, Lenti-viruses, Herpesviruses, Togaviruses or Retroviruses and microparticle bombardment such as by using DNA-coated tungsten or gold particles (Agacetus Inc., WI, USA).

Suitable prokaryotic cells for expression include corynebacterium, salmonella, Eicherichia coli, Bacillus sp. and Pseudomonas sp, amongst others. Bacterial strains which are suitable for the present purpose are known in the art (Ausubel et al, 1987; Sambrook et al, 2001).

Preferred mammalian cells for expression of the nucleic acid fragments include epithelial cells, fibroblasts, kidney cells, T cells, or erythroid cells, including a cell line selected from the group consisting of COS, CHO, murine 10T, MEF, NIH3T3, MDA-MB-231, MDCK, HeLa, K562, HEK 293 and 293T. The use of neoplastic cells, such as, for example, leukemic/leukemia cells, is contemplated herein.

Preferred mammals for expression of the nucleic acid fragments include, but are not limited to mice (ie., Mus sp.) and rats (ie., Rattus sp.).

The nucleic acid encoding the protein of interest, protein, binding partner, other protein or comprising a reporter gene can also be expressed in the cells of other organisms, or entire organisms including, for example, nematodes (eg C. elegans) and fish (eg D. rerio, and T. rubnipes). Promoters for use in nematodes include, but are not limited to osm-10 (Faber et al Proc. Natl. Acad. Sci. USA 96, 179-184, 1999), unc-54 and myo-2 (Satyal et al Proc. Natl. Acad. Sci. USA, 97 5750-5755, 2000). Promoters for use in fish include, but are not limited to the zebrafish OMP promoter, the GAP43 promoter, and serotonin-N-acetyl transferase gene regulatory regions

Placing the expression of a reporter genes operably under the control of an interaction To link reporter gene expression to a protein interaction, the protein of interest, the protein binding partner and any other protein must be expressed at the protein level, as described herein above. Additionally, the reporter gene must be operably linked to a suitable, promoter such that it is capable of being expressed to confer a detectable phenotype. Additionally, the expression of the reporter gene must be capable of being activated, by the binding of one protein to the upstream region of the reporter gene. (5′-UTR) and the interaction of that protein with its cognate binding partner.

Preferred promoters for driving reporter gene expression include those naturally-occurring and synthetic promoters which contain binding sites for transcription factors, more preferably for helix-loop-helix (HLH) transcription factors, zinc finger proteins, leucine zipper proteins and the like. Preferred promoters may also be synthetic sequences comprising one or more upstream operator sequences such as, for example, LexA operator sequences or activating sequences derived from any of the promoters referred to herein such as, for example, GAL4 DNA binding sites. Any of the promoters referred to supra are also suitable for driving reporter gene expression provided that they either naturally contain a suitable cis-acting regulatory sequence to which the protein of interest or the protein binding partner of the other protein can bind, or alternatively, have been engineered to contain such a site.

Preferably, the cis-acting sequence is selected from the group consisting of: LexA operator, GAL4 binding site, and cI operator. In accordance with this embodiment of the invention, it is preferred for the protein of interest or the protein binding partner or the other protein or a fusion protein comprising same to include a DNA binding domain capable of binding to said cis-acting sequence, in which case said DNA binding domain will be selected from the group consisting of: LexA operator binding domain, GAL-4 DNA binding domain; and cI operator binding domain, respectively.

Reporter genes are configured as described supra in a suitable gene construct. Suitably configured reporter genes are then introduced into a cellular host as described.

Host cells capable of expressing the variant protein of interest, and the native forms of the protein binding partner and other protein, and comprising the reporter genes necessary to perform the invention, are grown under conditions sufficient to enable the native form of the protein of interest to associate with the native form of the protein binding partner, and other protein. Conditions will also be selected that facilitate expression of the reporter genes, such as, for example, growth on a suitable media

The association of the variant protein of interest and the protein binding partner will reconstitute an active transcription factor that is capable of activating or enhancing expression of a reporter gene to which either protein docks. Similarly, the association of the variant protein of interest and the other protein will reconstitute an active transcription factor that is capable of activating or enhancing expression of a reporter gene to which either protein docks.

If both reporter genes are activated or enhanced then the mutation in the variant protein of interest is not within the interaction site of the protein of interest with either the protein binding partner or the other protein.

Conversely, if there is no expression of either reporter gene, then the mutation in the variant protein of interest is either a missense mutation encoding an allosteric change in conformation or a nonsense mutation introducing a STOP codon, or within the interaction site of the protein, of interest with both the protein binding partner and the other protein (i.e., the binding sites in the protein of interest for both proteins are either the same, contiguous, or overlap). In either case, such a phenotype is not useful unless the intention is to isolate allosteric mutants defining vulnerable residues to attack in screens for allosteric inhibitors.

In a preferred embodiment, there is expression of only one of the reporter genes, indicating that the mutation in the variant protein of interest is within the interaction site of the protein of interest with either the protein binding partner or the other protein. Accordingly, it is therefore possible to select for expression of a single reporter gene as being indicative that the mutation is within an appropriate binding site. This is made possible by the fact that formation of the different protein-protein interactions are distinguished by virtue of the operable, connection of the target interaction and the non-target interaction to distinct reporter genes, which can be assayed separately or simultaneously, depending upon the reporter genes used.

For example, distinct counter selectable reporter genes can be used, in which case the interactions can be distinguished by survival or growth of cells on particular substrates. In this respect, it is possible to distinguish between an interaction that is operably linked to both URA3 and CYH2 genes, and an interaction linked to the LYS2 gene. Cells in which an interaction is linked to expression of both URA3 and CYH2 genes are detectable, because they are resistant to fluororotic acid (5-FOA) and cycloheximide, and if those cells do not express LYS2, they will not require lysine for growth and/or are sensitive to growth on media containing α-aminoadipate (α-AA).

Similarly, it is possible to distinguish between interactions operably linked to distinct fluorescent protein-encoding reporter genes, by virtue of detecting the different emission wavelengths of the expressed proteins.

Selection of Cells

In accordance with the invention, cells expressing the variant protein of interest, protein binding partner and other protein, and expressing the reporter gene(s) operably connected to the interaction between the protein of interest and the other protein(s), but not expressing the reporter gene operably connected to the interaction between the protein of interest and protein binding partner or having a reduced level of expression thereof, are selected. In such cells, the interaction between the variant protein of interest and the protein binding partner is abrogated, whereas the interaction between the variant protein of interest and the other protein is not. Accordingly, the variant protein of interest will carry an informative mutation in the interaction interface, because it retains the ability of the native protein to interact with the other protein.

Selection of such cells will depend upon the reporter genes used, and can be readily performed using art-recognized procedures. Similarly, culture methods for growing bacterial yeast, or mammalian cells are well-known in the art.

In an alternative preferred embodiment of the invention, where the intention is to discover mutations which cause allosteric changes in folding of the target, cells are, selected and screened for mutations which reduce expression of reporter genes linked to both of the target interactions. Mutant proteins isolated from these yeast will then be expressed and assayed by Western blotting to ensure that the mutations isolated did not unduly effect efficient translation or stability of the protein.

### 2. Inhibitory Peptides

A second aspect of the present invention provides a method for determining an inhibitor of an interaction between a protein of interest and a protein binding partner in a cell, said method comprising:

expressing a mutated form of the protein of interest and the native form of the binding partner protein and native forms of one or more other proteins that bind to the protein of interest such that the binding of the mutated form of the protein of interest to the native form of the binding partner protein and each other protein operably controls the expression of a different reporter gene, and selecting for modified expression of the reporter gene that is operably under the control of a binding between the protein of interest and the binding partner protein and unmodified expression of each other reporter gene, wherein said modified expression indicates that the mutation is within a region in the protein of interest that mediates the ability of the protein to bind to the binding partner protein;

determining a fragment of the mutated form of the protein of interest said fragment comprising the region that mediates the ability of the protein to bind to the binding partner protein; and

determining a fragment in the native form of the protein of interest that is functionally equivalent to (b) wherein said fragment inhibits the interaction between the native form of the protein of interest and the binding partner.

Further steps available to those skilled in the art include the modelling of the position of the critical mutated residues in the tertiary structure of the target protein of interest, if the structure of this protein (or a closely related family member or orthologue) has been solved by standard structural techniques such as X-ray crystallography or Nuclear Magnetic Resonance Spectroscopy.

By “determining a fragment of the mutated form of the protein of interest” is meant that the variant form of the protein is recovered following selection and analysed to determine the nature of the mutation, such as, for example, by determining the nucleotide sequence of the. ORF that encodes it. Naturally, this will involve a comparison with the native nucleotide sequence. In such comparisons or alignments, differences will arise in the positioning of non-identical residues arising from insertion/deletions in the variant, depending upon the algorithm used to perform the alignment. Preferably, such alignments are made using software of the Computer Genetics Group, Inc., University Research Park, Maddison, Wis., United States of America, eg., using the GAP program of Devereaux et al., Nucl. Acids Res. 12, 387-395, 1984, which utilizes the algorithm of Needleman and Wunsch, J. Mol. Biol. 48, 443-453, 1970. Alternatively, the CLUSTAL W algorithm of Thompson et al., Nucl. Acids Res. 22, 4673-4680, 1994, is used to obtain an alignment of multiple sequences, wherein it is necessary or desirable to maximize the number of identical/similar residues and to minimize the number and/or length of sequence gaps in the alignment. Alignments can also be performed using a variety of other commercially available sequence analysis programs, such as, for example, the BLAST program available at NCBI.

Preferably, the sequences of several distinct variants of the protein of interest identified in a specific screen are aligned and compared, and more frequently-occurring alleles are determined. Alternatively, or in addition, less frequently-occurring alleles.

Additionally, determination of the length of the encoded variant protein, immunogenic cross-reactivity with the native protein, or a determination of the tertiary or quarternary structure of the variant protein can also be performed to obtain information on the nature and effect of the mutation. Such procedures are well within the ability of the skilled person and can be performed without undue experimentation.

By “determining a fragment in the native form of the protein of interest” is meant that an amino acid sequence in the native protein that encompasses all or part of the mutated site is identified. Such fragments are preferably short, comprising no more than about 50 amino acid residues and preferably no more than about 30 or 20 or 15 or 10 or 5 amino acid residues in length.

As will be apparent to the skilled person, preferred fragments of the native protein will retain the ability to bind to the protein binding partner and thereby have utility as an inhibitor or antagonist of the interaction between the protein of interest and the protein binding partner. Moreover, because such fragments are derived from the interaction site between those two proteins, they are highly specific and preferably do not adversely affect the interaction of the protein of interest with the other protein in vivo or in vitro.

Preferably, based upon the amino acid sequence of the determined fragment of the wild-type or native protein of interest, a peptide consisting of that sequence is synthesized using standard Fmoc/Boc chemistry as described in one or more of the following: J. F. Ramalho Ortigão, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, J. eds.), vol. 2, pp. 1-284, Academic Press, New York; Wünsch, E., ed. (1974) Synthese von Peptiden in Houben-Weyls Metoden der Organischen Chemie (Müler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474.

### 3. Use of the Interaction Interface to Validate Therapeutic Drug Targets

The recovered peptide comprising an interaction interface can be used to validate a therapeutic target (ie. it is used as a target validation reagent). By virtue of its ability to bind to a specific protein, it is well within the ken of a skill artisan to determine the in vivo effect of modulating the activity of the protein by expressing the identified peptide or protein domain in an organism (eg., a bacterium, plant or animal such as, for example, an experimental animal or a human). In accordance with this aspect of the present invention, a phenotype of an organism that expresses the identified peptide or protein domain is compared to a phenotype of an otherwise isogenic organism (ie. an organism of the same species or strain and comprising a substantially identical genotype however does not express the peptide). This is performed under conditions sufficient to induce the phenotype that involves the target protein or target nucleic acid. The ability of the peptide or protein domain to specifically prevent expression of the phenotype, preferably without undesirable or pleiotropic side-effects indicates that the target protein is a suitable target for development of therapeutic/prophylactic reagents.

Accordingly, a third aspect of the present invention provides a method for determining or, validating a protein interaction as a therapeutic drug target or validation reagent comprising:

expressing a mutated form of a protein of interest and the native form of a binding partner protein and native forms of one or more other proteins that bind to the protein of interest such that the binding of the mutated form of the protein of interest to the native form of the binding partner protein and each other protein operably controls the expression of a different reporter gene, and selecting for modified expression, of the reporter gene that is operably under the control of a binding between the protein of interest and the binding partner protein and unmodified expression of each other reporter gene, wherein said modified expression indicates that the mutation is within a region in the protein of interest that mediates the ability of the protein to bind to the binding partner protein;

determining a fragment of the mutated form of the protein of interest said fragment comprising the region that mediates the ability of the protein to bind to the binding partner protein;

determining a fragment in the native form of the protein of interest that is functionally equivalent to (b) wherein said fragment inhibits the interaction between the native form of the protein of interest and the binding partner; and

(d) expressing the fragment at (c) in a cell or organism and determining a phenotype of the cell or organism that is modulated by the target protein or target nucleic acid wherein a modified phenotype of the cell or organism indicates that the protein interaction is a therapeutic target or validation reagent.

Preferably, determining a phenotype of the organism that is modulated comprises comparing the organism to an otherwise isogenic organism that does not express the selected fragment. For example, the phenotype of an organism that expresses a tumor is assayed in the presence and absence of a peptide or protein domain that blocks an interaction between SCL and E47 in a screen of the expression library of the invention. Amelioration of the oncogenic phenotype by the expressed peptide indicates that the SCL/E47 is a suitable target for intervention, wherein the peptide is then suitably formulated for therapeutic intervention directly, or alternatively, small molecules are identified that are mimetics of the identified peptide or protein domain.

### 4. Mimetics of the Interaction Interface

A fourth aspect of the present invention provides a method for identifying a therapeutic or prophylactic compound comprising:

expressing a mutated form of a protein of interest and the native form of a binding partner protein and native forms of one or more other proteins that bind to the protein of interest such that the binding of the mutated form of the protein of interest to the native form of the binding partner protein and each other protein operably controls the expression of a different reporter gene, and selecting for modified expression of the reporter gene that is operably under the control of a binding between the protein of interest and the binding partner protein and unmodified expression of each other reporter gene, wherein said modified expression indicates that the mutation is within, a region in the protein of interest that mediates the ability of the protein to bind to the binding partner protein;

determining a fragment of the mutated form of the protein of interest said fragment comprising the region that mediates the ability of the protein to bind to the binding partner protein;

determining a fragment in the native form of the protein of interest that is functionally equivalent to (b) wherein said fragment inhibits the interaction between the native form of the protein of interest and the binding partner; and

identifying a mimetic compound of the fragment at (c).

Preferred methods for identifying mimetic compounds are based upon methods described in WO00/68373 and U.S. Ser. No. 10/372,003 for producing expression libraries of mimetic peptides or mimotopes known as “biodiverse gene fragments” (“BGF libraries”), which disclosures are incorporated herein by way of reference in their entirety. In these methods, the BGF libraries are screened to identify those peptides that have the same function as an isolated peptide derived from the protein of interest and comprising the interaction interface of that protein. Accordingly, the BGF libraries are screened to isolate those peptides that inhibit or abrogate the interaction between the protein of interest and the protein binding partner. Preferably, such mimotopes will not adversely affect the interaction of the protein of interest with another protein to which it binds in vivo.

Alternatively, random peptide (synthetic mimetic or mimotope) libraries are produced using short random oligonucleotides produced by synthetic combinatorial chemistry and screened for their ability to inhibit the interaction between the protein of interest and the protein binding partner.

To enhance the probability of obtaining useful bioactive mimetics from random peptide libraries, peptides can be constrained within scaffold structures, eg., thioredoxin (Trx) loop (Blum et al. Proc. Natl. Acad. Sci. USA, 97, 2241-2246, 2000) or catalytically inactive staphylococcal nuclease (Norman et al, Science, 285, 591-595, 1999), to enhance their stability. Constraint of peptides within such structures has been shown, in some cases, to enhance the affinity of the interaction between the expressed peptides and its target, presumably by limiting the degrees of conformational freedom of the peptide, and thereby mining the entropic cost of binding.

Mimotope libraries of up to several thousand polypeptides or peptides can be prepared by gene expression systems and displayed on chemical supports or in biological systems suitable for testing biological activity. For example, genome fragments isolated from Escherichia coli MG1655 can be expressed using phage display technology, and the expressed peptides screened to identify peptides that bind to the protein binding partner and inhibit interaction between the protein of interest and the protein binding partner, essentially as described by Palzkill et al. Gene, 221 79-83, 1998.

Additionally, mimotope libraries can be prepared essentially as described in U.S. Pat. No. 5,763,239 (Diversa Corporation), from uncharacterized environmental samples containing a mixture of uncharacterized genomes. The procedure described by Diversa Corp. comprises melting DNA isolated from an environmental sample, and allowing the DNA to reanneal under stringent conditions. Rare sequences, that are less likely to reanneal to their complementary strand in a short period of time, are isolated as single-stranded nucleic acid and used to generate a gene expression library. Again, the libraries are screened to identify proteins having the ability to bind to the protein binding partner and/or inhibit the interaction of the protein binding partner and the protein of interest eg., using reverse hybrid screens.

Alternatively, knowledge of critical residues required for the dimerisation of the target protein of interest with its partner gained from steps 4a-b above, can be applied to the rational design of peptoid or small molecule inhibitors which interact which such residues and block the interaction and/or folding of the target.

The present invention is further described with reference to the following non-limiting examples.

### EXAMPLE 1

### Developing Novel Therapeutic Leads Based Upon JNK MAPK Inhibitory Peptides

Introduction

This example describes new approaches to improve our understanding of specific inhibitors of the JNK MAPKs. These protein kinases, first described following their activation in response to stress, have been implicated in the intracellular events culminating in cell death. Because cell death underlies the pathologies of stroke and heart attack that are associated with the ischemia/reperfusion damage, the targeted inhibition of JNK promises an important therapeutic strategy.

Recently, an inventor described a small peptide inhibitor of JNK (MAB3), derived from an organiser/scaffold of the JNK pathway, designated “TI-JIP” (Truncated Inhibitor of JNK based on I). The inventors now have data supporting the efficacy of this inhibitor in protecting neuronal cells following ischemia/reperfusion. Data presented in FIG. 1 demonstrate that the cell-permeable TI-JIP maintains neuronal cell viability when applied either 1 hour before (denoted as TI-JIP in the Figure) or 1 hour after simulated stroke (denoted as TI-JIP 1 h in the Figure). Thus, this inhibitor does not require prior treatment for its efficacy. This is a critical finding because, although many other inhibitors have been tested and shown to be effective when used as pretreatments, the therapeutic intervention in stroke is possible only following the initial insult.

The inventors propose that inhibitors of JNK will provide an important strategy following ischemia/ reperfusion damage incurred in diseases such as stroke.

The inventors have continued to refine their understanding of the TI-JIP-JNK interaction, using a reverse two-hybrid screening technology described in WO99/35282, to map 3 critical residues of JNK, each of which prevents JNK interaction with TI-JIP when mutated.

Defining the Interaction Interface on Human JNK1 Using a Reverse Two Hybrid Assay

This example describes the identification and validation of critical residues of JNK that are required for the TI-JP-JNK interaction using a two hybrid assay. This defines amino acids of JNK that must be targeted by an effective and specific JNK inhibitor. This information is critical to the further development and/or discovery of JNK inhibitors targeting this site. The methods described herein have allowed the inventors to rapidly map, in less than 3 months, an interface on JNK that interacts with TI-JIP. This is faster than mapping by conventional co-crystallisation strategies, and reveals the interacting amino acids and the changes that interfere with binding.

Rationale

Following the identification of an effective peptide inhibitor of human JNK1, the inventors are now mapping the regions of JNK involved in this interaction. Improved knowledge of this interaction interface will allow the prediction and/or design of novel JNK inhibitors.

Broad Description of Approach and Results

The direct interaction of TI-JIP and human JNK1 by surface plasmon resonance has been demonstrated. TI-JIP inhibits JNK MAPK but not the closely-related p38 and ERK MAPKs. Four of the 11 amino acids of the TI-JIP, peptide are critical for its efficacy in vitro (MAB3).

The inventors have continued these studies, exploiting the power of yeast screening approaches, to confirm the TI-JIP-JNK interaction and its disruption by single amino acid substitution in TI-JIP. These results highlight the specificity of interactions in the JNK-TI-JIP interface.

To demonstrate that interaction interface can be mapped in a yeast system, the inventors have now exploited reverse two-hybrid screening systems described in WO99/35282.

The inventors constructed a JNK1-mutant library using random PCR mutagenesis. Using TI-JIP in the bait vector, yeast were selected in a single step as described herein for growth on selective media indicating the failure of TI-JIP to interact with mutant JNKs. The significant advance in these protocols has been the introduction of a galactose-titratable expression of the interacting partners thereby allowing greater discrimination of the interactors through continuous adjustment of screening stringency. Full-length JNK mutants were then sequenced.

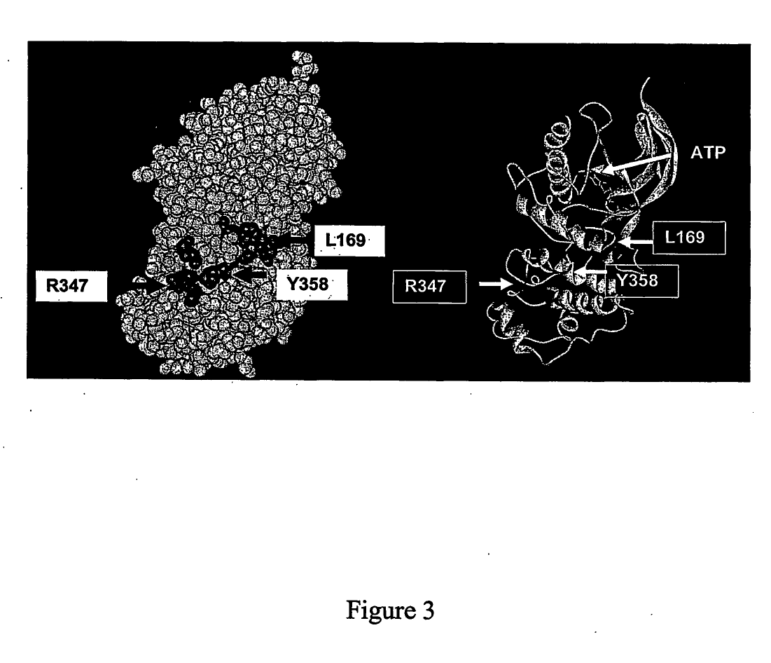

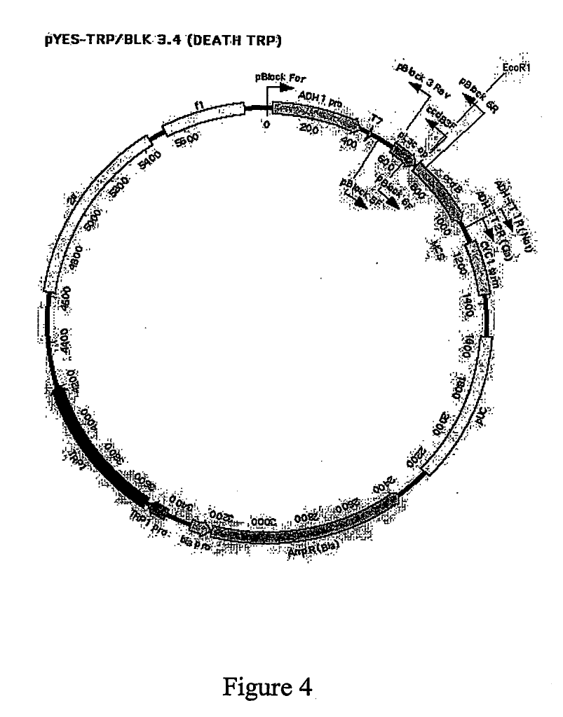

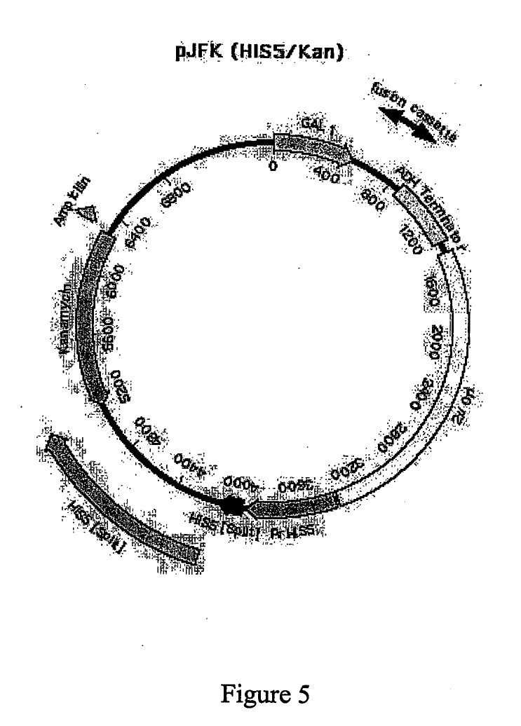

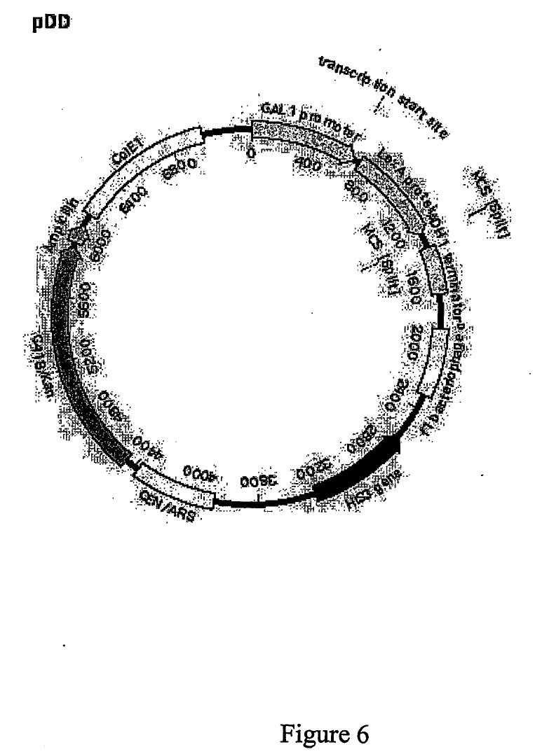

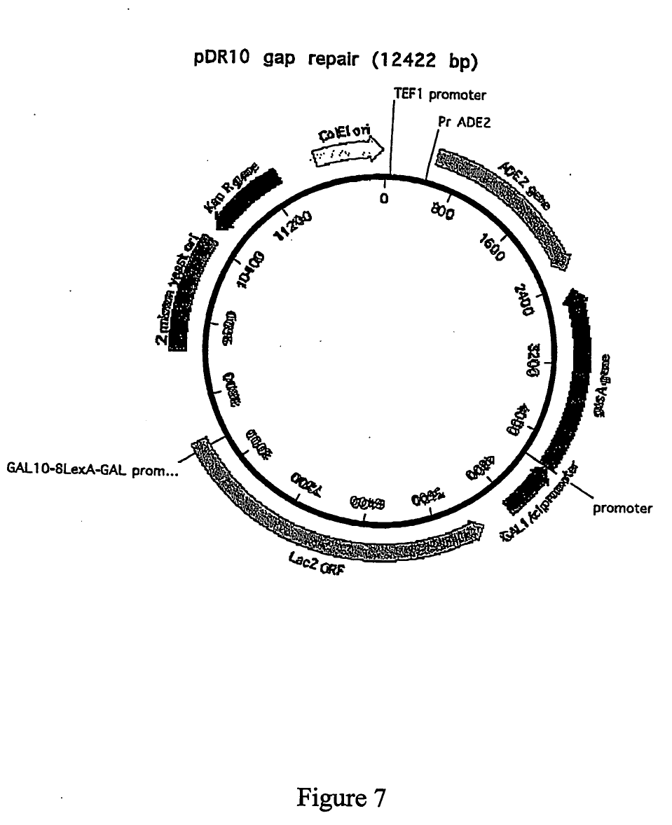

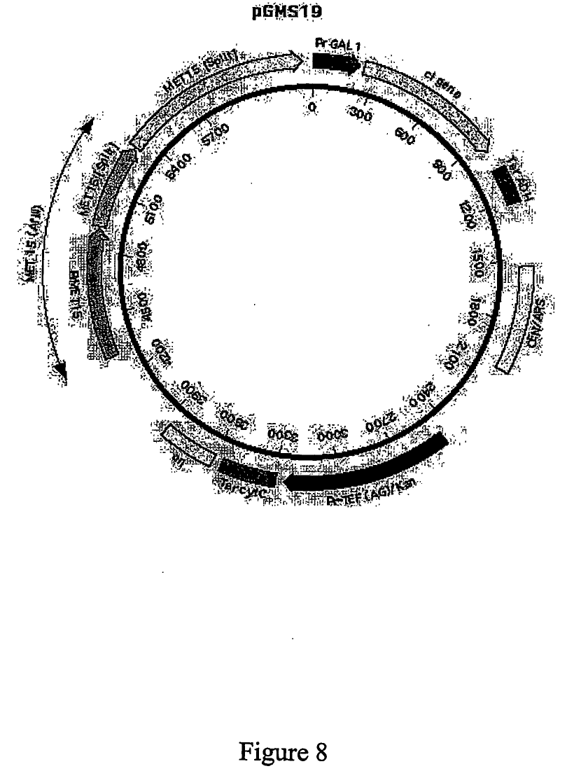

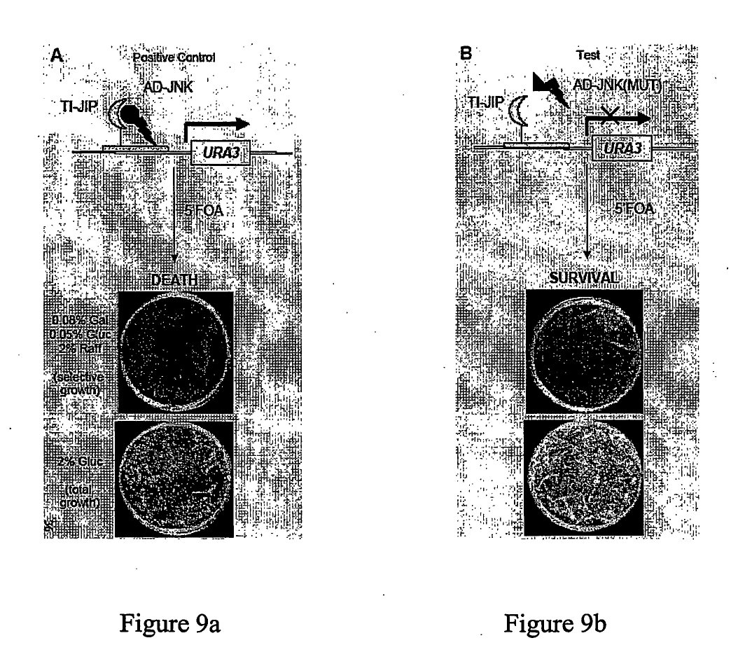

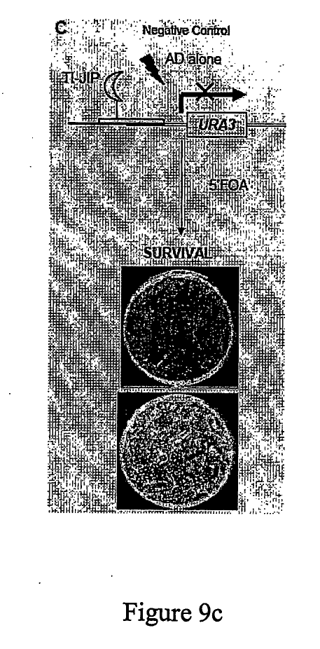

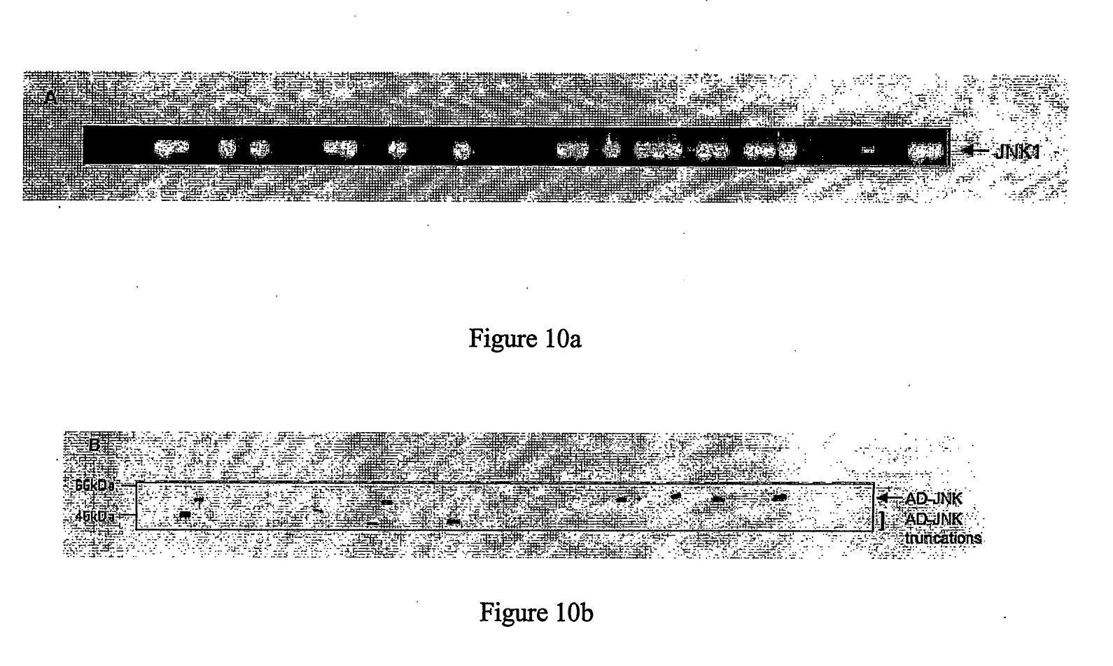

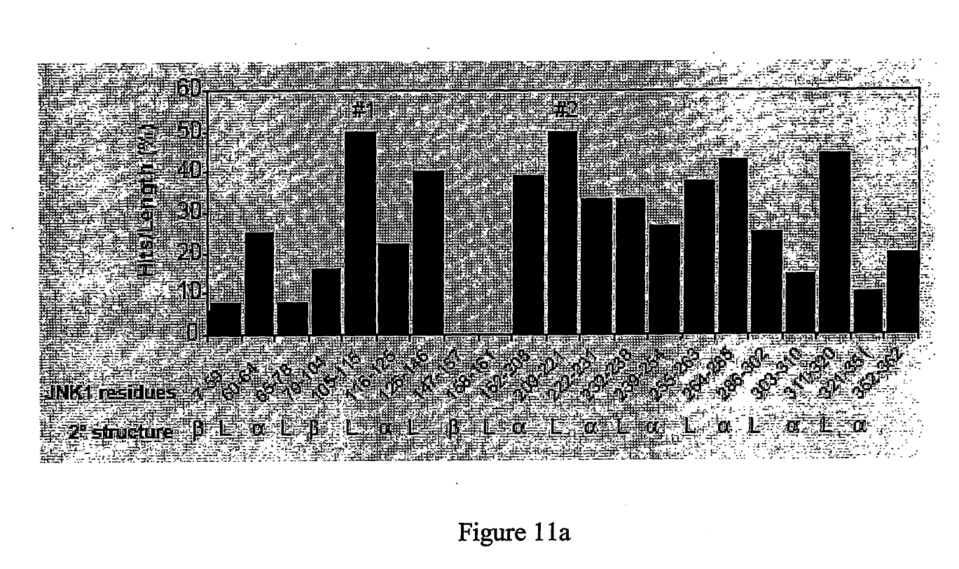

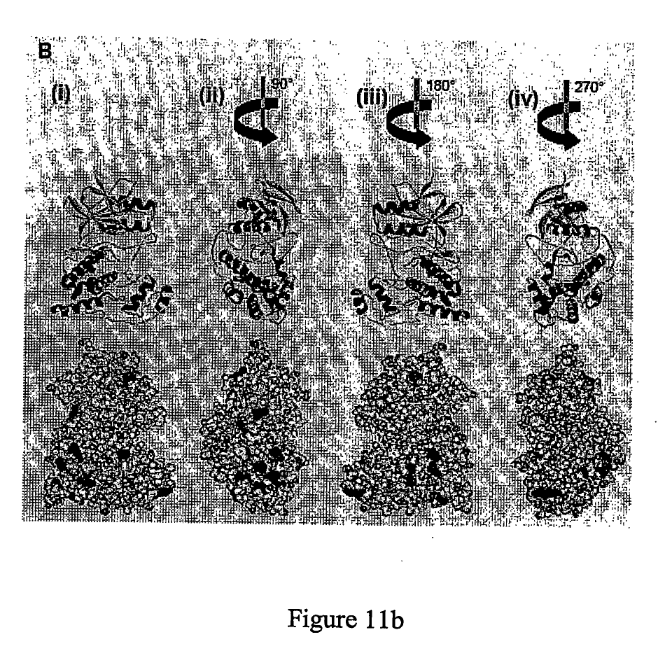

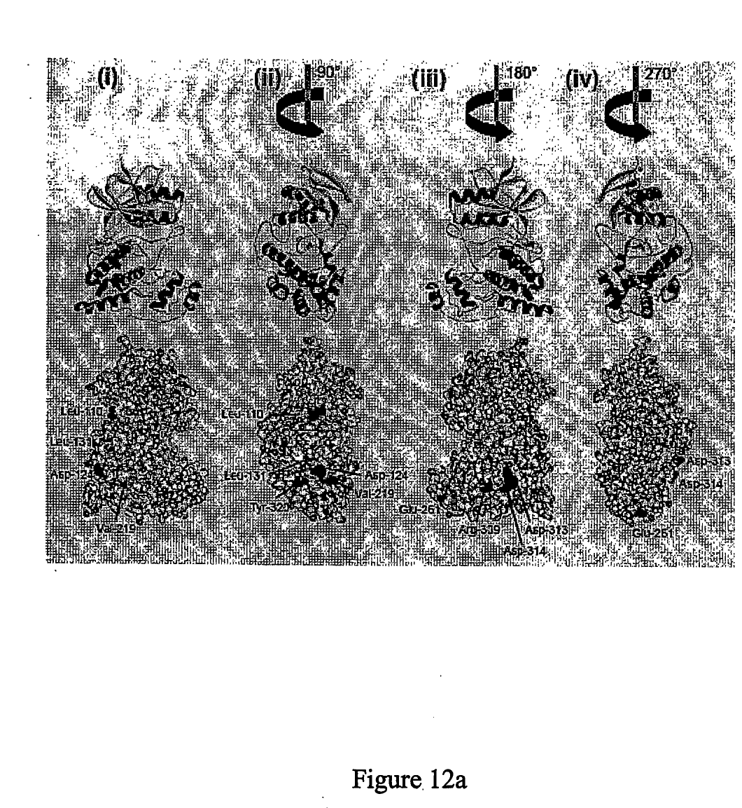

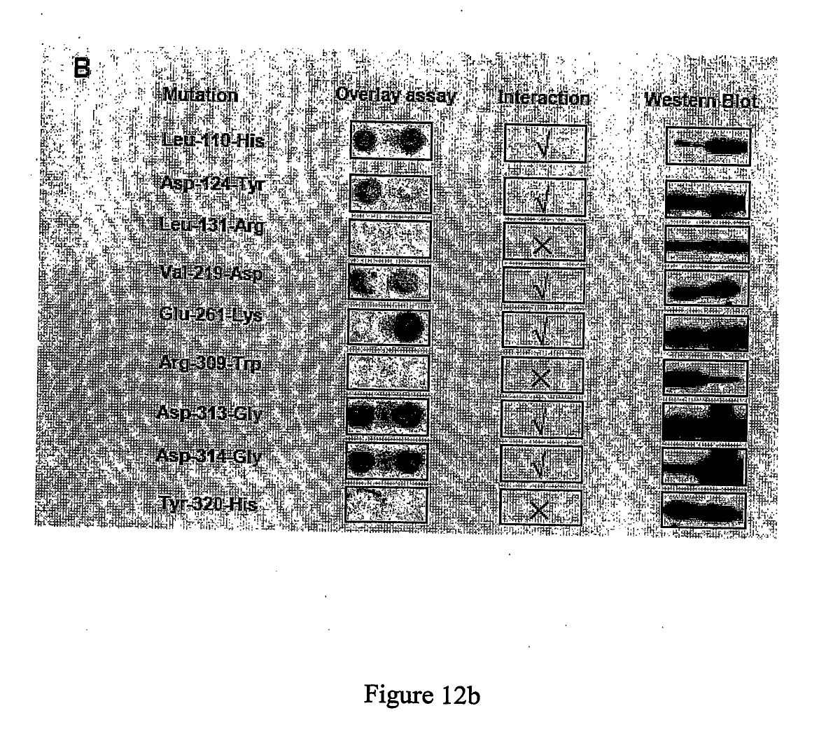

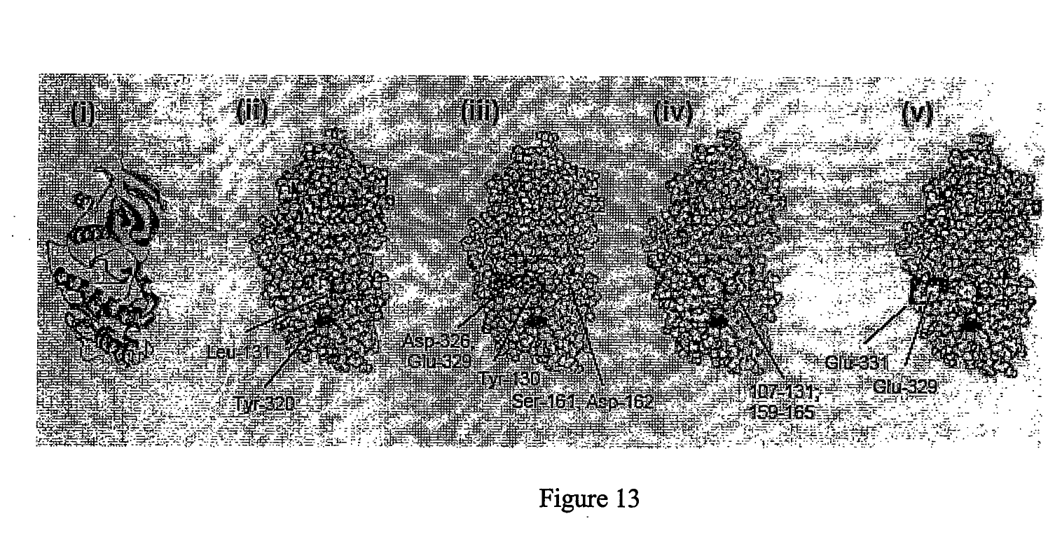

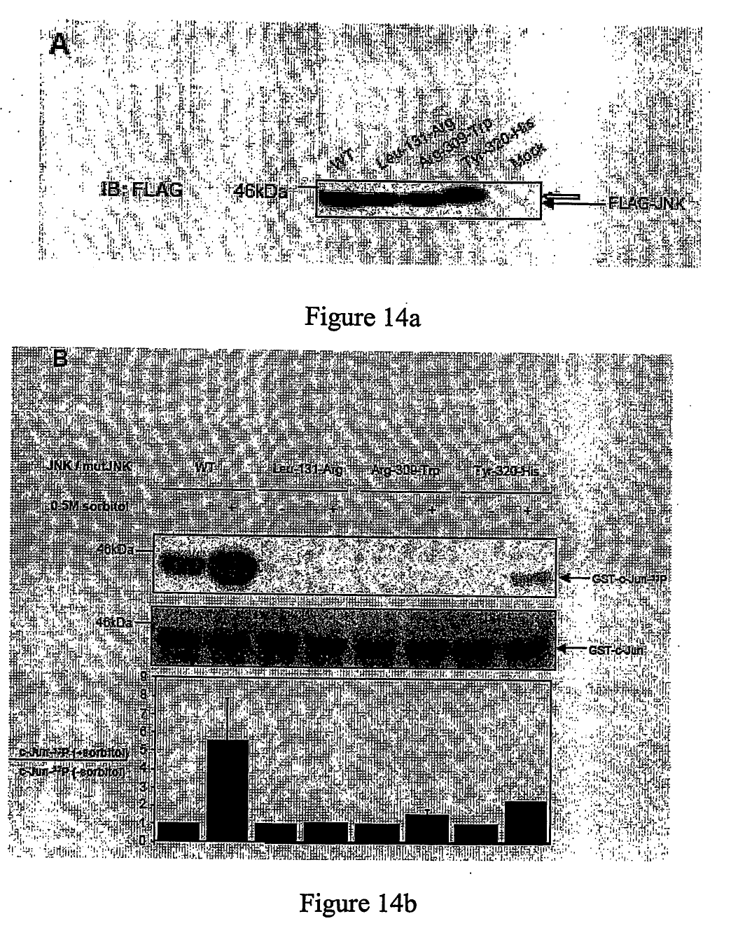

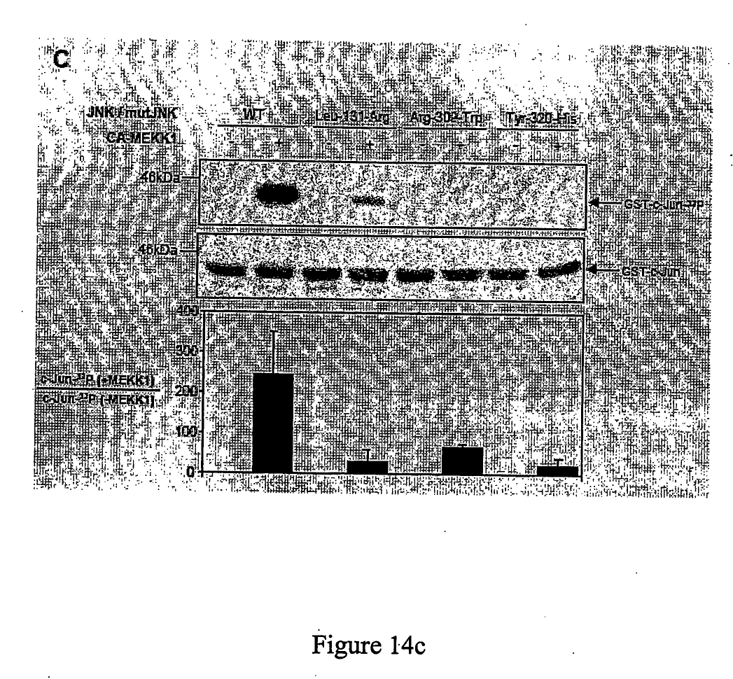

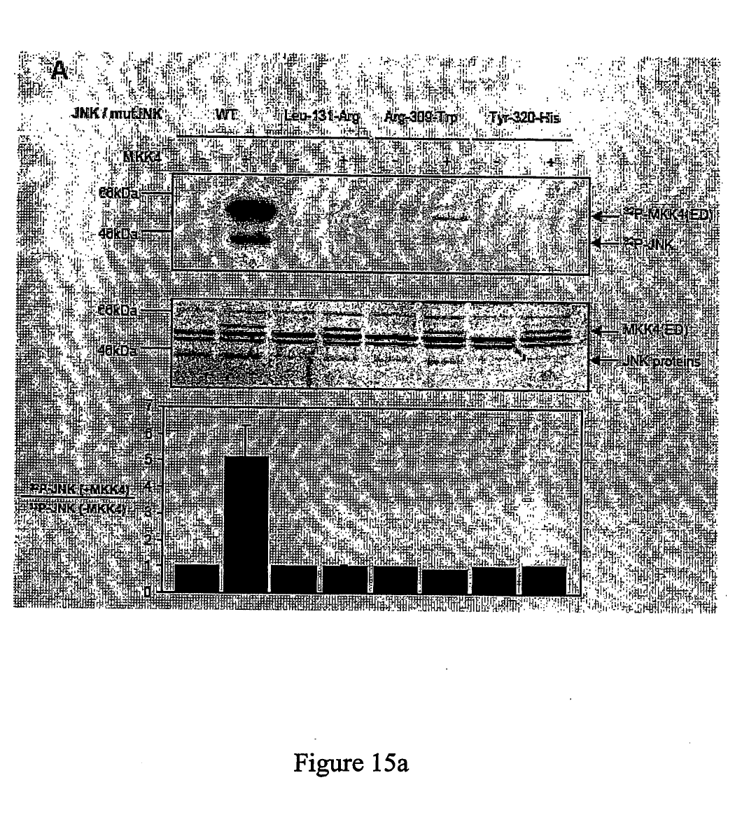

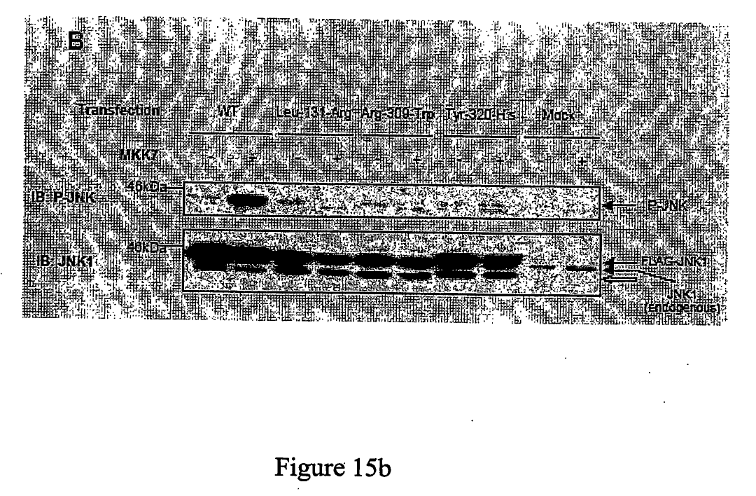

From a first screen of 0.6×10⁶ diploids, the inventors evaluated six INK mutants. The inventors have subsequently shown that three amino acid residues in JNK, as highlighted in FIG. 3, are required for the TI-JIP-JNK interaction. In particular, the mutations L131→R131, R309→W309, and Y320→H320, prevent interaction of JNK with TI-JIP.

To further refine the JNK-TI-JIP interface, surface-exposed residues that are within the linear sequence between R309 and Y320 of SEQ ID NO: 1 are evaluated (i.e., the amino acid sequence ³⁰⁹RISVDEALQHPY³²⁰). Alternatively, or in addition, sequences flanking this region is evaluated. In particular, multiple JNK mutant libraries are created by site-directed mutagenesis of individual residues to create changes at the following residues:

I311, D313, E314, Q317, P319, K300, W324, E126, and S129.

For each residue, a NN[T/C] codon is introduced to thereby produce a mutated form of a JNK1 protein wherein all amino acids are represented at these positions, with the exception of Q, E and W. Degenerate oligonucleotide pairs are used separately, to create a series of mutant JNK libraries enriched in changes in the region of the proposed interface. This strategy was selected over alternative approaches, such as, for example, the introduction of the degenerate codon NNN, to ensure that a premature translation termination codon is not introduced into the gene, thereby encoding a truncated JNK1 protein. Background is further minimized because there is no carry-through of empty vector. No amino acid residues buried in the kinase domain of JNK1 are mutated.

To confirm that the mutations in JNK1 produce a TI-JIP-resistant JNK sparag, selected mutants are expressed as FLAG-JNK fusion proteins in a mammalian expression vector. Following transient transfection in HEK293 cells, constant expression levels of these mutants is confirmed by immunoblotting with FLAG antibody. Immunoprecipitates of control and mutant forms of FLAG-JNK from stimulated cells are obtained. The activity of control and mutant forms of FLAG-JNK from stimulated cells towards the transcription factor c-Jun is evaluated, along with the activation/phosphorylation of those proteins, such as, for example, by immunoblotting with a phospho-JNK antibody.

Each JNK mutant is tested to ensure that it is not inhibited by TI-JIP. These immunoprecipitation and kinase assays are standard procedures.

Experimental Methods

Plasmid DNA Constructs

Oligonucleotides encoding TI-JIP were annealed to produce a fragment with ends compatible “with EcoRI at the 5′ end and XhoI at the 3′ end. These were ligated into the pGILDA vector (CLONTECH), which had been digested with EcoRI/XhoI, thus generating C-terminal fusion proteins with the LexA DNA-binding domain. The human JNK1 sequence (SEQ ID NO: 1) was PCR-amplified and then digested with MfeI and XhoI. The use of MfeI, which is an isoschizomer of EcoRI, avoided internal digestion within the JNK1 sequence but produced the required sticky ends for subsequent cloning. These fragments were ligated into the pJG4-5 vector (CLONTECH), which had been digested with EcoRI/XhoI, to produce C-terminal fusion proteins with the B42 transcriptional activation domain. DNA sequencing confirmed the identity of these constructs.

Construction of Mutant JNK Library Using Random PCR Mutagenesis

Reactions (50 μL) containing 5U Taq polymerase (ROCHE), 50 pmol forward primer, 50 pmol reverse primer and 10 ng template DNA in Error-prone PCR buffer (final concentrations: 100 mM Tris-HCl pH 8.3, 500 mM KCl, 70 mM MgCl₂, 0.1% (w/v) gelatin, 10% (v/v) DMSO, 0.2 mM DATP, 0.2 mM dGTP, 1 mM dCTP, 1 mM dTTP) were performed in 0.25mL PCR tubes. A total of four different mutagenesis reactions were performed, where MnCl₂ was added to final concentrations of 0.1 mM, 0.2 mM or 0.3 mM prior to temperature cycling, or MnCl₂ was added to a final concentration of 0.3 mM following completion of 10 rounds of temperature cycling. Reactions were subjected to 30 cycles with the following conditions: [94° C. for 1 min; 55° C. for 1 min; 72° C. for 3 min]. Following thermal cycling, reactions were pooled and digested with MfeI/XhoI. The digested products were ligated into EcoRI/XhoI-digested pJG4-5, transformed into ElectroTenBlue™ (Stratagene) electrocompetent E. coli and plated on LB agar containing 100 μg/mL ampicillin. Plates were incubated at 30° C. overnight, then placed at 37° C. for three hours to allow maximum growth of a total of 9×10⁶ single well-isolated colonies. The bacterial library was harvested and DNA was isolated using a QIAGEN Maxiprep Kit. This was introduced into the yeast strain PRT 48, which was derived from the strain SKY 48 (MATα, trp1, ura3, his3, 6lexAop-LEU2, cIop-LYS2) (Serebriiski et al., J. Biol. Chem 274, 17080-17087, 1999) in accordance with the Gietz High Efficiency Transformation Protocol (Agatep, et al., Technical Tips Online 1998), and yeast were grown at 30° C. for 4 days on synthetic complete medium lacking tryptophan and containing 2% Glucose. The resulting 5×10⁵ single well-isolated colonies were harvested and stored at −80° C. in sterile Yeast Freezing Buffer (65% (v/v) glycerol, 0.1 M MgSO₄, 25 mM Tris-HCl pH 8.0).

Interaction Mating

The yeast strain PRT 480 (MATa, his3, trp1, ura3, 4 LexA-LEU2, lys2::3 cIop-LYS2, CAN^{R}, CYH2^{R}, ade2::2 LexA-CYH2-ZEO, his5::2 LexA-URA3-G418) was constructed from the SKY 473 yeast strain provided by Ilya Serebriiskii, Fox Chase Cancer Center. We introduced into strain PRT 480 the bait plasmid, pGILDA-TI-JIP. We then mated these transformants to PRT 48, which carried either pJG4-5-JNK1, the mutant JNK1 library was constructed in pJG4-5, or the pJG4-5 vector control. In each mating, the total number of cells was 3×10⁸ with a bait:prey ratio of 5:1. Thus, 2.5×10⁸ colony forming units of bait were mated with 5×10⁷ colony forming units of prey. Yeast were resuspended in 200 μL Yeast Extract Peptone Dextrose (YPD) liquid medium (10 g/L Yeast extract, 20 g/L Peptone, 20 g/L Glucose, 20 g/L Bacto-Agar) and then plated on 90 mm YPD agar plates and grown at 30° C. for 12-15 h. Diploids were harvested, washed in sterile H₂O and plated on reverse screening plates.

Reverse Two-Hybrid Screening to Isolate JNK1 Mutants That Lost the Ability to Interact with TI-JIP

PRT 480/PRT 48 diploids expressing either pGILDA-TI-JIP/pJG4-5-JNK (positive control), pGILDA-TI-JIP/pJG4-5 (negative control) or pGILDA-TI-JIP/pJG4-5-mutant 10 JNK library (test) were plated at densities of 150,000 diploids per 90 mm plate of synthetic complete medium lacking uracil, histidine and tryptophan (HI) agar plate containing 2% (w/v) Raffinose (Raff), 0.05% (w/v) Glucose (Gluc), 0.08% (w/v) Galactose (Gal) and 0.07% (w/v) 5′fluoroorotic acid (5′FOA). Plates were supplemented with uracil (final concentration of 0.02 mg/mL) to support the growth and 15 survival of yeast prior to any reporter activation. In this novel reverse two hybrid screening system, the screening threshold can be adjusted by modulating the level of sugars in the media. These optimized screening conditions provided maximal death of positive control yeast with minimal death of negative control yeast. Plates were incubated at 30° C. for 72 h, after which time colonies were clearly visible. 20.

Characterisation of Non-Interacting JNK Mutants

Yeast expressing JNK mutants that did not interact with TI-JIP were plated on HW agar containing 2% (w/v) Glucose and grown at 30° C. These yeast were then replica plated onto synthetic complete agar lacking leucine (L agar) containing either 2% (w/v) Gluc, or 0.08% (w/v) Gal and 2% (w/v) Raff, and incubated at 30° C. for 72 h to test for the interaction between JNK and TI-JIP using forward two-hybrid analysis. This control forward analysis was possible due to the 6lexAop-LEU2 reporter carried by the yeast strain PRT 48. Colonies were regarded as false positives if they grew on the L Gal/Raff plates, which indicated an interaction between the mutant JNK protein and TI-JIP. Genuine non-interactors were grown on HW agar containing 0.05% (w/v) Gal and 2% (w/v) Raff for 48 h at 30° C., then vortexed in 20 μL SDS-PAGE Sample

Buffer and snap-frozen in liquid N₂. Samples were heated at 100° C. for 5 min prior to separation by SDS-PAGE. Proteins were transferred to nitrocellulose by semi-dry electroblotting and probed for HA-tagged products. Yeast found to express a full-length HA-tagged activation domain-JNK1 fusion protein (58 kDa) were expanded in HW liquid medium containing 2% Gluc, and JNK constructs were rescued by lyticase extraction. These were electroporated into KC8 bacteria, plated on LB agar containing 100 μg/mL ampicillin and grown overnight at 37° C. Colonies were then plated on M9 agar lacking tryptophan (4 g/L Glucose, 1× M9 salts (64 g/L Na₂HPO₄.7H2O, 15 g/L KH₂PO₄, 2.5 g/L NaCl, 5 g/L NH₄Cl), 2 mM MgSO₄, 0.1 mM CaCl₂ and 0.75 g/L amino acid dropout mix lacking tryptophan (Ausubel et al ibid.) containing 50 μg/mL kanamycin and grown at 30° C. for 48 h. Mutant pJG4-5-JNK DNA was isolated using a QIAGEN Spin Miniprep Kit prior to sequencing and analysis of mutations. A pool of 16 mutant JNK sequences was identified, each containing from 2 to 11 mutations in the full length JNK sequence. In total, 70 amino acids had been mutated and some mutations were common to more than one mutant JNK sequence.

Identification of Mutational “Hot-Spots” on JNK

From the pool of 16 JNK mutants, the frequency of mutations per region of secondary structure of JNK was calculated and normalized for the length of the structure. This resulted in the identification of secondary “hot-spots”. The mutations were also mapped onto the surface of the JNK3 structure (PDB: 1JNK) using WebLab ViewerLite software. This indicated that some mutations that appeared distant in the protein primary structure were close to each other in the tertiary structure, resulting in tertiary “hot-spots”. To reduce noise, the mutant pool was reduced to those containing five or less point mutations per JNK protein. We then chose nine such regions to target by point mutation, and constructed these point mutants using the Stratagene QuikChange protocol. These were screened for interaction with TI-JIP using forward two-hybrid screening and, β-galactosidase overlay assays (described below). Western immunoblotting for the HA-tagged mutant JNK proteins was performed as described above to confirm that full-length JNK proteins were expressed from the mutant constructs. Point mutants of JNK1 that did not interact with TI-JIP were constructed in the pCMV-FLAG-JNK1 using the Stratagene QuikChange protocol to assess their biochemistry in mammalian cells.

β-Galactosidase Overlay Assays

The RFY 206 strain (MATa, trp1, ura3-52, his3-200, leu2-3, lys2-Δ201, trp1::hisG) carrying the pSH18-34 lacZ reporter plasmid and pGILDA-TI-JIP was mated to the PRT 49 strain derived from the SKY 48 strain (MATα, trp1, ura3, his3, 6-lexAop-LEU2, 3-cIop-LYS2, ade2) carrying JNK mutants in pJG4-5. For qualitative analysis of β-galactosidase activity, these diploids were replica plated onto UHW agar containing either 2% (w/v) Gluc, or 2% (w/v) Raff and 0.05% (w/v) Gal. Following incubation at 30° C. for 48 h, protein-protein interactions were assessed using the chloroform overlay assay technique (adopted from Duttweiler et al., Trends Genet. 12, 340-341, 1996). Yeast grown on agar plates were overlaid with chloroform and incubated at room temperature for 5 min. Plates were then rinsed with chloroform, dried upside down for 5 min, then overlaid with a solution of 1% low-melting agarose in 100 mM potassium phosphate buffer, pH 7.0, containing X-Gal at a concentration of 1 mg/mL. Once the agarose solidified, plates were incubated at 30° C. and monitored for 20 min-3 h for colour changes; Protein-protein interactions were monitored via lacZ reporter, activity converting the colourless X-Gal substrate to a coloured product.

Cell Transfection, Lysis and Immunoblotting

COS cells were transfected with pCMV-FLAG-JNK1 (Derijard et al., Cell 76, 1025-1037, 1994) or equivalent mutant constructs and pEBG-MKK7β1 (provided by A. Whitmarsh, University of Manchester) as specified in the Figures using Lipofectamine and PLUS reagent (Invitrogen) according to the manufacturer's instructions. Following cell, lysis as described in Barr et al., J. Biol. Chem 277, 10987-10997, 2002) and addition of 3× SDS Sample Buffer, proteins were separated using SDS-PAGE. Following protein transfer onto nitrocellulose, immunoblotting was performed using either anti-active JNK (Promega), anti-FLAG M2 (SIGMA) or anti-JNK1 (Santa-Cruz) primary antibodies. Primary antibodies were bound by horseradish peroxidase-conjugated secondary antibodies (PIERCE) and immunocomplexes were visualized using chemiluminescence.

Immunoprecipitation and Protein Kinase Assays

FLAG-JNK1 proteins were immunoprecipitated by addition of anti-FLAG M2 (SIGMA) and incubation for 1 h on ice and then addition of Protein G-Sepharose and incubation at 4° C. for 2 h with rotation. Immunocomplexes were washed three times with lysis buffer, then once with reaction buffer (20 mM HEPES, 20 mM MgCl₂, 20 mM β-glycerophosphate, 500 μM DTT, 100 μM Na₃VO₄; pH 7.6). For assays of JNK activity, the washed complexes were resuspended in 40 μL of reaction buffer containing 10 μg GST-c-Jun (1-135), 20 μM ATP and 1 μCi [λ-³²P]ATP and incubated at 30° C. for 30 min. Reactions were stopped by addition of 3× SDS-PAGE Sample Buffer and proteins were separated by SDS-PAGE. JNK activity towards GST-c-Jun (1-135) was visualized by autoradiography and quantitated by Cerenkov counting. For assays of JNK activation, the washed beads were incubated with 30 μL of reaction buffer containing 20 μM ATP, 5 μCi [λ-³²P]ATP and 1 μg of GST-MKK4(ED) at 30° C. for 1 h with occasional mixing. After removal of the supernatant, the beads were washed in 200 μl ice-cold lysis buffer and then heated for 5 min at 100° C. in 15 μL of 3× SDS-PAGE sample buffer, prior to separation by SDS-PAGE. Gels were Coomassie-stained, dried and used for autoradiography. Gel bands corresponding to FLAG-JNK were excised from the gels and their radioactivity quantitated by Cerenkov counting. Where immunoprecipitated GST-MKK7β1 was used to activate JNK, reactions were performed as above, but with 1 μCi [λ-³²P]ATP and incubation at 30° C. for 30 min.

Results

Random PCR Mutagenesis Created a Library of JNK Mutants

Initially, we constructed a series of directed N- and C-terminal truncations of JNK as fusions with the C-teriminus of the GAL4 transcriptional activation domain, to identify a smaller region of JNK to be subjected to mutagenesis. However, these JNK mutants were poorly expressed in RFY 206/PRT 49 diploids relative to the wild type protein (data not shown), and therefore we proceeded to randomly mutagenise the entire JNK1 sequence. In optimizing the random PCR mutagenesis, we found that reactions containing 0.3 mM MnCl₂ resulted in the presence of up to 11 point mutations per full length JNK sequence. Therefore, we used four different mutagenic PCR conditions to generate a library of JNK sequences containing up to 11 point mutations per JNK sequence.

Reverse Two-Hybrid Screening

We employed a reverse two-hybrid method to screen the library of JNK mutants for those that lost the ability to interact with TI-JIP (FIG. 9). In this system, the PRT 480 yeast strain with the counterselectable URA3 reporter gene was transformed with pGILDA-TI-JIP. These yeast were mated to PRT 48 yeast transformed with the mutant JNK library in the pJG4-5 vector, and grown in the presence of 5′fluoroorotic acid (5′FOA), which is toxic to yeast when the URA3-encoded enzyme is expressed. In the presence of Galactose (Gal), the neutral carbon source Raffinose (Raff) and a low concentration of Glucose (Gluc) to reduce background survival (upper panels), bait and prey expression was induced and yeast expressing interacting partners were sensitive to 5′FOA. Therefore, in the presence of 5′FOA, an interaction between TI-JIP and JNK resulted in cell death (FIG. 9a). In contrast, a lack of interaction between TI-JIP and either a non-interacting JNK mutant (FIG. 9b) or the activation domain encoded by the empty pJG4-5 vector (FIG. 9c) allowed yeast to survive treatment with 5′FOA. More yeast colonies grew on the test plates (TI-JIP plus mutant JNK library; FIG. 9b) than the positive control plates (TI-JIP plus JNK; FIG. 9a), but this was less than the number on the negative control plates (TI-JIP plus pJG4-5; FIG. 9c), which would be expected when the mutant JNK library contained both non-interacting mutants and mutants which were phenotypically normal and retained the ability to interact with TI-JIP. Yeast were separately grown in the presence of Glucose, which repressed bait and prey expression resulting in insensitivity to 5′FOA and was indicative of the total number of viable yeast on the plates.

Analyzing Colonies That Survived the Reverse Two-Hybrid Screening

Approximately 600 colonies were obtained after plating 600,000 diploids on the. reverse screening plates. Screening by colony PCR using JNK-specific primers indicated that 200 of the 600 colonies contained a prey plasmid with a JNK-insert. A representative selection of this screen is shown in FIG. 10a. Immunoblotting for the HA-tagged prey protein indicated that only 21 of the 200 interaction-deficient mutants expressed a full-length JNK protein (46 kDa) in fusion with the activation domain, AD (12 kDa) to produce the expected protein size of 58 kDa. A representative selection of this screen by immunoblotting showing 6 full-length JNK proteins and 4 truncated JNK proteins is shown in FIG. 10b. The full-length JNK proteins were further analysed 5 of the 21 colonies were found to represent by forward two-hybrid screening to confirm that they did not interact with TI-JIP, and false positives because they did interact with TI-JIP under the conditions of the forward screen (results not shown). It is likely that these false positive yeast grew on the reverse screening plates in spite of interacting bait and prey proteins that would normally produce toxicity and death or due an evasion of the counter selection pathway such as the epigenetic shutdown of the URA3 reporter expression in the yeast. The 16 remaining interaction-deficient mutants were analysed by DNA sequencing, to determine the mutations present in the corresponding JNK proteins.

Summary of Mutation Data

From the reduced pool of 16 mutants, the frequency of mutations per region of secondary structure was calculated and normalised for the length of the structure (FIG. 11a), resulting in secondary “hot-spots”. Although the structure of the JNK1 protein has not been solved, human JNK1 and JNK3 demonstrate up to 96% sequence homology when their sequences are compared using an Entrez BLAST query. Therefore the JNK1 mutations were also mapped onto the surface of the JNK3 structure to depict their positions in the protein tertiary structure. Because the mutations mapped to various regions of the JNK structure, it was difficult to detect tertiary “hot-spots”. Therefore, we reduced the mutant pool to those containing 5 or, less point mutations per JNK molecule in an attempt to reduce background noise. This reduced the mutant pool from 16 to 6. Furthermore, this revealed some clustering of mutations on the surface of JNK, particularly in the C-terminal lobe of JNK (FIG. 11b). Using both the secondary and tertiary “hot-spot” data along with residues that were altered in multiple mutants, we assigned regions for further investigation.

We chose 9 individual JNK residues to target by point mutation. Using site-directed mutagenesis, we altered single residues of JNK to represent the changes that occurred in mutants isolated by reverse two-hybrid screening. Specifically, the point mutations were Leu-110-His, Asp-124-Tyr, Leu-131-Arg, Val-219-Asp, Glu-261-Lys, Arg-309-Trp, Asp-313-Gly, Asp-314-Gly and Tyr-320-His. Locations of the targeted residues are represented on the JNK1 protein structure in FIG. 12a. When these mutants were tested for interaction with TI-JIP by forward two-hybrid screening, a β-galactosidase overlay assay indicated that of the nine point mutants tested, only the Leu-131-Arg, Arg-309-Trp and Tyr-320-His did not interact with TI-JIP (FIG. 12b). This was not simply due to impaired protein expression of the mutants, because western blotting indicated that full-length JNK proteins were expressed (FIG. 12b). Two independent yeast colonies were tested in the case of each mutation to confirm the results of the β-galactosidase overlay assay and western blotting.

The residues Leu-131 and Tyr-320 were located near each other on a common face of the JNK protein (FIG. 12a (ii)), whereas Arg-309 mutation was located on another face of JNK (FIG. 12a (iii). These amino acids were not buried within the core of the JNK protein, and therefore it is unlikely that their mutation affected the global folding or stability of the protein (Jiang et al., In: Protein phosphorylation—A practical Approach, Oxford University Press Inc, New York pp 315-333, 1999). Because these residues demonstrated some surface exposure, it was possible that they were involved in mediating the interaction between JNK and TI-JIP. In addition, because TI-JIP is a KIM-based peptide, it was possible that these mutations would disrupt the interaction between JNK and other KIM-containing proteins. To investigate this notion, we compared the locations of JNK1 residues Leu-131, Arg-309 and Tyr-320 with the locations of other regions proposed to mediate the interactions between MAPKs and KIMs.

Proximity of JNK1 Residues Leu-131, Arg-309 and Tyr-320 to Regions of APKs Previously Reported to Interact with Kinase Interaction Motifs (KIMs)

The acidic “CD” domain of MAPKs is characterized by negatively charged amino acids and is located on the opposite side to the active site in the structure of MAPKs Tanoue et al., EMBO J, 20, 466-479, 2001). In human JNK1, the CD domain residue Asp-326 is conserved, and the acidic Glu-329 might also be considered part of the domain. JNK1 residues Leu-131 and Tyr-320 (FIG. 13 (ii)) are situated on a common face of the kinase to these CD residues, but not directly adjacent to these residues (FIG. 13 (iii)). In addition to the classical “CD” site residues, other ERK2 CD residues have been identified that are responsible for high affinity MKP3 binding (Zhang et al., J. Biol. Chem 278, 29901-29912, 2003). The JNK1 residue Tyr-130 shares homology with a corresponding residue in ERK2 reported to be involved in the ERK2-MKP3 interaction, and it is located directly adjacent to Leu-131, identified herein (FIG. 13 (iii)). The “ED” and “TT” residues in p38 and ERK2, respectively are equivalent to JNK1 residues Ser-161 and Asp-162 (“SD” site). This site on JNK is situated on the same face of the kinase as the CD domain, and Leu-131 is situated directly below the “SD” site residues (FIG. 13 (iii). Therefore, although Leu-131 and Tyr-320 are distinct from both the CD site and the ED sites, they are located on the same face of JNK as these regions and are situated relatively close to these sites.

The co-crystallisation of p38 MAPK in complex with KIM-based peptides from substrate MEF2A and activator MKK3b identified a site in the C-terminal domain of the kinase thought to participate in hydrophobic interactions with the KIM peptides (Chang et al., Mol. Cell. 9, 1241-1249, 2002). The equivalent regions of human JNK1 comprise Val-107 to Leu-131 and Val-159 to Leu-165. The JNK1 residue Leu-131 is situated directly within this cluster of residues, and Tyr-320 is situated directly adjacent to these residues (FIG. 13 (iv)). In addition, Ile-116 in p38 was reported to form hydrophobic contacts with the L-X-L motif present in the KIM consensus sequence (Chang et al., Mol. Cell. 9, 1241-1249, 2002), and the side-chain of the corresponding JNK1 residue, Val-118, points towards Leu-131 and is in close proximity to this residue (3-5 Å). Finally, the p38 residues Leu-113 and Leu-122 were also found to be in contact with bound KIM peptides (Chang et al., Mol. Cell 9, 1241-1249, 2002). These residues are conserved in p38, ERK2 and JNK1/2, and in JNK1 their side chains are also in close proximity to Leu-131 (4 Å).

Whilst our study was nearing completion, it was reported that JNK2 residues Glu-329 and Glu-331 were important for the interaction between JNK2 and JIP-1 (Mooney et. al., J. Biol. Chem. 279, 11843-11852, 2004). In particular, Glu-329 was critical for efficient binding between JNK2 and JIP-1, whereas Glu-331 made a more minor contribution. These residues are conserved in JNK1, and Leu-131 and Tyr-320 are situated a short distance (12-14 Å) from Glu-329 (FIG. 13 (v)). Therefore, it is feasible that these residues could all contribute to the formation of a docking groove that binds the JIP-1 KIM.

In summary, at least Leu-131 and Tyr-320 are situated relatively close to regions of MAPKs thought to mediate interactions with KIMs of interacting partners. Therefore, it seemed possible that in addition to disrupting the JNK-TI-JIP interaction, these mutations would disrupt the interactions between JNK and other KIM-containing partners. To further investigate this hypothesis, we investigated the biochemistry of these JNK1 mutants in mammalian cells.

JNK1 mutants were impaired in their ability to phosphorylate c-Jun following exposure to activating stimuli.

We constructed the Leu-131-Arg, Arg-309-Trp and Tyr-320-His point mutants of JNK1 in the pCMV-FLAG vector for mammalian expression. COS cells were transfected with these constructs, and Western blotting performed on cell lysates revealed over-expression of FLAG-tagged JNK1 and all three tagged mutants FIG. 14a). In addition, the Tyr-320-His mutant consistently demonstrated reduced mobility following SDS-PAGE relative to the wild-type protein, despite DNA sequencing of the construct ensured that no other mutations were present (FIG. 14a).

We then tested the activation of these JNK proteins by two different stimuli. Hyperosmotic shock (0.5 M sorbitol, 30 min) is a well-described activator of mammalian JNK (Bogoyevitch et al., J. Biol. Chem. 270, 297100-29717, 1995). Exposure of COS cells transfected with wildtype JNK1 to 0.5 M sorbitol for 30 min resulted in strong phosphorylation of c-Jun substrate in in vitro kinase assays using FLAG-immunoprecipitation from lysates prepared from these cells, which corresponded to 5.5-fold activation over the corresponding unstimulated cells (FIG. 14b). However, a lower level of stimulation of c-Jun phosphorylation was detected in kinase assays of FLAG-immunoprecipitates from lysates of sorbitol-stimulated COS cells individually transfected with mutant JNKs, corresponding to only 1-2 fold over the corresponding unstimulated samples (FIG. 14b).

When a constitutively-active form of MEKK1 (CA-MEKK1) was co-transfected into COS cells with wildtype JNK, FLAG immunoprecipitates from these cell lysates displayed a 240-fold increase in c-Jun phosphorylation in in vitro kinase assays relative to the sample prepared from cells transfected with JNK alone (FIG. 14c). However, the corresponding samples with mutant JNKs displayed, a much lower amount of c-Jun phosphorylation (20-70 fold) following co-transfection of CA-MEKK1, relative to samples prepared from cells transfected with the JNK mutants alone (FIG. 14c). Therefore, the JNK mutants displayed an impaired ability to phosphorylate c-Jun in response to both of these activating stimuli.

JNK1 Mutants Were Not Activated by Either MKK4 or MKK7

The impaired c-Jun phosphorylation by the JNK mutants (FIGS. 14a and 14b) may have resulted from their impaired activation, impaired ability to bind substrate, or a combination of these factors. To clarify this issue, we directly investigated the phosphorylation of these mutants without relying on the subsequent phosphorylation of c-Jun. Mutant JNKs were immunoprecipitated from transfected cell lysates and incubated with a constitutively active form of MKK4 (GST-MKK4(ED)) in the presence of [λ-³²P]-ATP. The presence of active MKK4 increased the phosphorylation of wildtype JNK relative to the autophosphorylation that occurred in the absence of any upstream activator protein (FIG. 15a). In contrast, the negligible amount of radioactive phosphate incorporated into any of the three JNK mutants was not increased by the presence of active MKK4 (FIG. 15a). In addition, there appeared to be some phosphorylation of GST-MKK4 in the assay, and it was evident that this was increased in the presence of wildtype JNK, but not in the presence of any of the JNK mutants (FIG. 15a).

Whilst our study was nearing completion, it was reported that a double alanine mutant of JNK2 (Glu-329-Ala, Glu-331-Ala) did not interact with JIP-1, c-Jun or MKK4, but retained the ability to be activated by MKK7 (Mooney et al., J. Biol. Chem. 279, 11843-11852, 2004). Therefore, we tested the ability of the Leu-131-Arg, Arg-309-Trp and Tyr-320-His JNK1 mutants to be activated by MKK7, by phospho-blotting and in vitro kinase assays. Phospho-blotting for dual-phosphorylated JNK indicated that wild-type JNK1 was strongly phosphorylated by MKK7 in co-transfected cells (FIG. 15b, upper panel), whereas co-transfection of MKK7 did not result in phosphorylation of any of the JNK mutants (FIG. 15b, upper panel). This was despite the over expression of these JNK mutant proteins relative to endogenous JNK as indicated by Western blotting for total JNK1 (FIG. 15b, lower panel). Similar results were obtained from in vitro kinase assays, where wild-type JNK was strongly phosphorylated in the presence of MKK7, but no detectable phosphorylation of the JNK mutants occurred in the presence of MKK7 (data not shown). In addition, like the assays with JNK and MKK4, the presence of wild-type JNK in the assay stimulated the phosphorylation of MKK7, but the presence of mutant JNKs did not stimulate the phosphorylation of MKK7. Therefore, it appeared that the Leu-131-Arg, Arg-309-Trp and Tyr-320-His JNK1 mutants were impaired in their activation by both MKK4 and MKK7, contributing to their impaired responses to hyperosmolarity and co-transfection with CA-MEKK1 (FIG. 14).

Discussion

The JNK MAPK pathway is activated following exposure of cells to a wide range of extracellular stimuli including stress, cytokines and growth factors, but still the role that JNK activation plays remains controversial (reviewed by Bogoyevitch et al., Biochim. Biophys, Acta 1697, 89-101, 2004). Our understanding of this pathway is being enhanced by multiple parallel approaches including gene knockouts and over expression studies, as Well as closer evaluation of the biochemical features of members of this pathway. In addition to studies on the JNKs themselves, or their upstream activators, increasing attention is focused on the regulation of JNK signaling by the JIP family of scaffold proteins. Interestingly, JIPs have been reported to both increase (Whitmarsh et al., Science 281, 1671-1674, 1998) and decrease (Barr et al., J. Biol. Chem 277, 10987-10997, 2002; Bonny et al., Diabetes 50, 77-82, 2001; Dickens et al., Science 277, 693-696, 1997) signaling through the JNK cascade.

We have further investigated the binding interaction between JNK and the TI-JIP peptide, which represents the KIM of the JIP-1 scaffold protein. Using reverse hybrid analysis of a library of mutant JNK1 proteins, we isolated mutant JNKs that lost the ability to interact with TI-JIP. By constructing individual point mutations to assess the relative importance of putative mutational “hot-spots” on the JNK1 protein, we implicated the” residues Leu-131, Arg-309 and Tyr-320 as mediators of the interaction between JNK1 and TI-JIP.

Although site-directed mutagenesis and co-immunoprecipitation analysis are effective for a relatively small number of mutations and for targeting a well-defined region, for many interactions, the potential binding interface is poorly defined. In such cases, mutations targeting many surfaces of the protein can be made and a relatively large number of mutants screened. Random PCR mutagenesis allows the generation of a relatively large pool of mutants; and yeast two-hybrid or N-hybrid assays provide an efficient technique for screening these mutants for non-interactors. The efficiency advantage of reverse two-hybrid and N-hybrid screening over conventional forward two-hybrid screening is that the reverse screening selects against an interaction from up to 10 million mutants, whereas forward two-hybrid screening selects for an interaction. The result of this is that non-interactors are easily obtained with reverse hybrid screening, whereas more extensive forward hybrid screening is required to isolate non-interactors.

It is interesting to note the CD and ED site residues previously reported to mediate the docking interactions between MAPKs and interactors were not involved in the interaction between JNK and JIP-1. This was demonstrated by Mooney et al., J. Biol. Chem. 279, 11843-11852, 2004, who showed that mutation of the CD site residue Glu-326 to asparagines did not disrupt the JNK-JIP-1 interaction, despite its location directly adjacent to Glu-329, which was deemed critical for this interaction. In addition, although the ED site was reported to regulate the specificity of docking interactions for ERK and p38 MAPKs mutation of the region spanning Lys-160 to Asp-162, along with the residue Thr-164 within this site, did not disrupt the JNK—JIP-1 interaction (Mooney et al., J. Biol. Chem. 279, 11843-11852, 2004). It does appear, however, that the residues that mediate JNK binding to JIP-1/TI-JIP are also involved in the interactions of JNK with other activators and substrates, given that the JNK1 mutants in our study were not efficiently activated by MKK4 or MKK7, and that JNK2 mutants that do not bind JIP-1 are not activated by MKK4 and cannot bind c-Jun (Mooney et al., J. Biol. Chem. 279, 11843-11852, 2004). This emphasizes the notion that KIMs bind to similar regions of MAPKs via a combination of both common and distinct binding determinants.

### EXAMPLE 2

### Validation of Inhibitors of the JNK1/TI-JIP Interaction Using a Reverse Three Hybrid Assay With Dual Baits

Chang et al., J. Biol. Chem 278, 9195-9202, 2003 showed that murine WOX1 and human WOX3 interact with human JNK1 via the WW domain in the N-terminus of the WOX protein, however human WOX3 protein appears to promote higher endogenous activation of gene expression than murine WOX1. This is presumably due to the presence of an activation domain in WOX3 that is produced as consequence of the deletion in WOX3 that truncates and modifies the C-terminus of the protein relative to WOX1.

The interaction interface between TI-JIP and JNK1 (Example 1) is confirmed using a reverse three hybrid assay PCT/US01/07669). The binding partners assayed are JNK (SEQ ID NO: 1) and TI-JIP (SEQ ID NO: 4) as described in the preceding example, and a WOX protein selected from the group consisting of human WOX3 (SEQ ID NO: 17), human WOX1 (SEQ ID NO: 18) and murine WOX3 (SEQ ID NO:. 19). Alternatively, or in addition, multiple WOX proteins are separately assayed in conjunction with the JNK1/TI-JIP proteins in a reverse three hybrid assay.

In particular, the dual fluorescent reporter construct pRT2 (SEQ ID NO: 14) is transformed into a yeast strain that requires adenine, thereby conferring adenine auxotrophy and enabling selection for maintenance of the vector. Nucleic acid encoding TI-JIP is cloned into the vector pDD (SEQ ID NO: 13) to yield the plasmid pDD-TI-JIP. Nucleic acid encoding a WOX protein is cloned into the plasmid pGMS19 (SEQ ID NO: 15) to yield pGMS19-WOX. Yeast cells carrying the dual reporter gene construct pRT2 are then transformed with pDD-TI-JIP and pGMS19-WOX to thereby express TI-JIP as a fusion with the LexA DNA binding domain, and a WOX protein as a fusion with the DNA binding domain of cI. This yeast is then mated to yeast cells transformed with the mutant JNK library in the pJFK vector (SEQ ID NO: 12). Yeast grown in media lacking adenine, histidine and methionine. Expression of all binding :partners is induced in the presence of Galactose (Gal), the neutral carbon source Raffinose (Raff) and a low concentration of Glucose (Gluc) to reduce background. Yeast cells are assayed by FACS for expression of the GFP and cobA proteins, and yeast cells expressing the red fluorescent protein (cobA) but not the green fluorescent protein (GFP) are selected. The amino acid sequences of the mutant JNK1 proteins in the selected yeasts are determined and compared to the sequences identified in Example 1. The identification of mutations at Leu-131, Arg-309 and Tyr-320 confirms the validity of the assay system. In contrast to the reverse two hybrid assay described in the preceding example, the incidence of uninformative mutations is reduced in a single step.

### EXAMPLE 3

### Identification of TI-JIP Mimetic Compounds

This example describes the identification of mimetic compounds of TI-JIP that are identified in a screen of a BGF library derived from biodiverse microbial genomes created and validated as described in U.S. Ser. No. 10/372,003. With this BGF library, the inventors will identify new peptides utilizing the JNK-TI-JIP interface. Data already obtained with this library suggests that the encoded peptides yield 10 to 1000-fold better hit rates than the best rates reported from comparable screens of random peptides in aptamer libraries. Using in vitro assays, the inventors will confirm the ability of peptide mimotopes to inhibit JNK and prevent neuronal apoptosis. Non-peptide small inhibitor molecules of JNK are also identified. The technologies used are broadly applicable to emerging approaches to target protein-protein interaction interfaces in general. Novel peptides that also the TI-JIP/JNK interface are identified using the screening approaches described herein to screen BGF libraries.

Screening of 10% of a 2×10⁶ BGF library using Discriminating Blocker Trap reverse two-hybrid technology as described in U.S. Ser. No. 10/372,003 has successfully isolated peptides that block the SCL/E47 interaction but do not bind to either SCL or E47 in the proteins from which they were derived (i.e., in their native context). These peptides also do not block related interactions. (SCL/E2.2 and E47/ID). The peptide fragments range in size from 15 to 29 amino acids, and showed no sequence homology. This suggests conserved structural motifs that are responsible for the inhibition observed.

To select for peptides that block the JNK/TI-JIP interaction, a dual-bait reporter system described herein is used. In this system, the conditional toxicity of the URA3 gene product (in the presence of 5-fluoro-orotic acid) and the CYH2 gene product (in the presence of cycloheximide) allows selection of non-interacting bait and prey. A LacZ reporter is also used to reduce background. Thus, mimetic peptides in the BGF library that block the TI-JIP/JNK interaction permit cell survival in the presence of both 5-fluoro-orotic acid and cycloheximide, and colonies of these cells remain white in medium comprising the chromogenic substrate X-Gal. An added advantage of this approach is the modulation of screening stringency via a galactose-inducible bait/prey expression system. Mimetic peptides having different affinities for the JNK-TI-JIP interface are selected by varying the galactose concentration, with screening under the most stringent conditions identifying the blockers of highest-affinity. About 25 mimetic peptides are identified from a primary screen of about 1×10⁶ clones.

Peptides are synthesized by Auspep Ltd., Australia. For each mimetic peptide, a glycine-spacer and Biotin label are included at the N-terminus, to facilitate subsequent validation testing. For example, this labelling facilitates a determination of the JNK binding cabability of each peptide, using BIAcore surface plasmon resonance.

Each peptide is also tested for its ability to inhibit JNK activity towards c-Jun, and other substrates including Elk1 and ATF-2, using established methods. A range of peptide concentrations (0.001 to 10 μM) is tested.

Using these protocols, the JNK inhibitory properties of 89 peptides based on TI-JIP have been assessed.

In parallel, inhibitory activity of the mimetic peptides toward ERK or p38 MAPKs is determined and those peptides that do modify these pathways are eliminated.

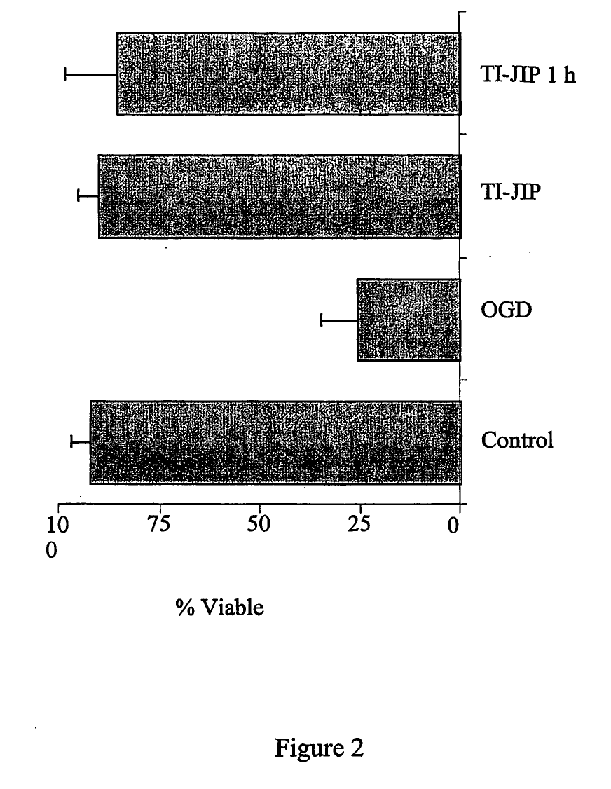

JNK-inhibitory mimetic peptides are delivered to neuronal cells using protein tranduction domain (PTD) technologies. Each peptide is synthesised with the TAT-PTD and a fluorescent FITC label at its N-terminus. Cultured neurons are preincubated with TAT-conjugated peptides (2 μM), exposed to oxygen-glucose deprivation (OGD) to simulate stroke, then maintained in normal medium for 24 h. Cell death is assessed by DAPI staining, with apoptotic cells showing fragmented nuclei, necrotic cells having condensed nuclei, and the nuclei of viable cells being only faintly stained. As shown in FIG. 2, control cultures are 90% viable, with this decreasing to 25% when cells are subjected to oxygen glucose deprivation (OGD). JNK-inhibitory peptides that are at least as active as TAT-TI-JIP (i.e. maintaining≧80% viability of neurons) are also evaluated at lower doses. Those with higher affinity for JNK are effective at lower doses.

Using data obtained for the mimetic peptides and data on residues important for TI-JIP interactions with JNK, together with the published X-ray crystallographic structure of JNK, the modelling tools Deep View, Wit!P (Novartis) and QXP (as available from Colin McMartin, Thistlesoft Software Co., USA), simulated docking of inhibitory peptides is performed. Deep View defines the binding surface on JNK and ensures consistency between the generated models and experimental results. Refinement utilizes the more powerful programs Wit!P and QXP.

The docked peptides in silico define key binding cavities for inhibitors on the surface of JNK. In the second phase of screening, small non-peptidic, drug-like molecules that have atoms or groups of atoms corresponding to key binding elements of the inhibitory peptides are obtained from database screens and their ability to inhibit JNK activity is determined.

Inhibitors are designed and docked into the JNK model structure. Monte Carlo docking of low molecular weight compounds into the defined binding site is performed with QXP and DOCK, and modeling of the best candidates refined with Wit!P. The leads are refined using the classical optimisation procedures of medicinal chemistry as shown by King In: Medicinal Chemisty-Principles and Practice, 2^{nd} Edition, Royal Soc. Chemistry, 2002.

1. A method for identifying a region in a protein of interest that mediates the ability of the protein to bind to a binding partner protein in a protein complex that comprises more than two proteins, said method comprising expressing a mutated form of the protein of interest and the native form of the binding partner protein and native forms of one or more other proteins that bind to the protein of interest such that the binding of the mutated form of the protein of interest to the native form of the binding partner protein to each other protein operably and separately controls the expression of a different reporter gene, and selecting for modified expression of the reporter gene that is operably under the control of a binding between the protein of interest and the binding partner protein and unmodified expression of each other reporter gene, wherein said modified expression indicates that the mutation is within a region in the protein of interest that mediates the ability of the protein to bind to the binding partner protein.

2-3. (canceled)

4. The method according to claim 1 wherein modified expression consists of a reduced expression of a reporter gene relative to the expression of the reporter gene in the presence of a native form of the protein of interest and a native form of the binding partner protein and wherein said method comprises determining reduced expression of the reporter gene in a forward hybrid assay wherein binding between the protein of interest and the binding partner activates expression of a reporter gene and wherein reduced expression of the reporter gene indicates that a mutation in the mutated form of the protein of interest is within a region of the protein of interest that mediates the ability of the protein of interest to bind to the binding partner protein.

5-12. (canceled)

13. The method according to claim 1 wherein modified expression consists of a reduced expression of a reporter gene relative to the expression of the reporter gene in the presence of a native form of the protein of interest and a native form of the binding partner protein and wherein said method comprises determining reduced expression of the reporter gene in a reverse hybrid assay wherein binding between the protein of interest and the binding partner activates expression of a counter selectable reporter gene encoding a polypeptide that is capable of reducing cell growth or viability by providing a target for a cytotoxic or cytostatic product or by converting a substrate to a cytotoxic or cytostatic product and wherein reduced expression of the counter selectable reporter gene enhances cell growth or viability thereby indicating that a mutation in the mutated form of the protein of interest is within a region of the protein of interest that mediates the ability of the protein of interest to bind to the binding partner protein.

14. (canceled)

15. The method according to claim 1 wherein the protein of interest and the binding partner protein are the same protein or allelic variants of the same protein.

16. The method according to claim 1 wherein the binding partner protein and other protein are allelic variants or mutant forms or orthologues of the same protein.

17. The method according to claim 1 wherein the protein of interest and/or the protein binding partner and/or the other proteins is/are expressed as a fusion protein.

18. The method according to claim 17 wherein the protein of interest, the protein binding partner and the other proteins are each expressed as a fusion protein.

19-37. (canceled)

38. The method according to claim 1 further comprising expressing a native form of the protein of interest and the native form of the binding partner protein and native forms of one or more other proteins that bind to the protein of interest such that the binding of the native form of the protein of interest to the native form of the binding partner protein to each other protein operably and separately controls the expression of a different reporter gene, and determining expression of each reporter gene.

39. (canceled)

40. The method according to claim 1 further comprising producing a mutated from of the protein of interest.

41. The method of claim 40 wherein producing a mutated form of the protein of interest comprises mutating a nucleotide sequence encoding the protein of interest or a fragment thereof such that the encoded peptide varies by one or more amino acids compared to nucleic acid encoding the native form of the protein of interest.

42. The method of claim 41 wherein nucleic acid encoding the protein of interest or a fragment thereof is modified by a process of mutagenesis selected from the group consisting of mutagenic PCR, replicating the nucleic acid in a bacterial cell that induces an accumulation of a random mutations through defects in DNA repair, site directed mutagenesis, and replicating the nucleic acid in a host cell exposed to a mutagenic agent.

43. The method of claim 42 wherein mutagenic PCR is performed by a process selected from the group consisting of: (i) performing the PCR reaction in the presence of manganese; and (ii) performing the PCR in the presence of a concentration of dNTPs sufficient to result in misincorporation of nucleotides.

44. A method for identifying a region in a protein of interest that mediates the ability of the protein of interest to bind to a protein binding partner in a protein complex that comprises the protein of interest and the protein binding partner and one or more other proteins, said method comprising the steps of:
(i) providing a cell that comprises: (a) a nucleic acid comprising a counter-selectable reporter gene encoding a polypeptide that is capable of reducing cell growth or viability by providing a target for a cytotoxic or cytostatic compound or by converting a substrate to a cytotoxic or cytostatic product, said gene being positioned downstream of a promoter comprising a cis-acting element such that expression of said gene is operably under the control of said promoter and wherein a fusion protein comprising the protein binding partner binds to said cis-acting element; (b) nucleic acid comprising a reporter gene other than the counter-selectable reporter gene of (a) positioned downstream of a promoter comprising the cis-acting element other than the cis-acting element at (a) such that expression of said reporter gene is operably under the control of said promoter and wherein a fusion protein comprising the other protein binds to said cis-acting element; (c) nucleic acid encoding a fusion protein comprising a variant or mutated form of the protein of interest and an activation domain that, activates expression of reporter genes (a) and (b); (d) nucleic acid encoding encoding a fusion protein that comprises the protein binding partner fused to a DNA binding domain of a transcription factor that binds to the cis-acting element in the counter selectable reporter gene (a) such that when the protein binding partner binds to the variant or mutated form of the protein of interest expression of the counter-selectable reporter gene at (a) is enhanced; and (e) nucleic acid encoding a fusion protein that comprises the other protein fused to a DNA binding domain of a transcription factor that binds to the cis-acting element in the reporter gene (b) such that when the other protein binds to the variant or mutated form of the protein of interest expression of the reporter gene at (b) is enhanced;
(ii) culturing said cell for a time and under conditions sufficient for the reporter genes at (i)(a) and (i)(b) and the fusion proteins at (i)(c), (i)(d) and (i)(e) to be expressed and for a native form of the protein of interest to bind to the protein binding partner and to the other protein;
(iii) culturing the cell in the presence of the substrate or the cytotoxic or cytostatic compound such that the expressed counter-selectable reporter gene reduces the growth or viability of the cell unless said expression is inhibited or reduced by virtue of the variant or mutated form of the protein of interest having reduced binding to the protein binding partner;
(iv) culturing the cell under conditions sufficient to detect expression of the reporter gene at (i)(b) by virtue of an interaction between the variant or mutated form of the protein of interest and the other protein;
(v) detecting expression of the reporter genes at (i)(a) and (i)(b); and
(vi) selecting or screening for a cell that expresses the reporter gene at (i)(b) and has reduced or inhibited expression of the reporter gene at (i)(a) compared to a cell that expresses the native form of the protein of interest, wherein the selected cell carries a mutation in a region in the protein of interest that mediates the ability of the protein of interest to bind to the protein binding partner.

45. The method of claim 44 wherein providing a cell comprises introducing nucleic acid into a cell that encodes at least one protein selected from the group consisting of the protein of interest, the protein binding partner, and the other protein.

46. The method of claim 44 wherein providing a cell comprises introducing nucleic acid that comprises a reporter gene downstream of a promoter that comprises a cis-acting element to which the protein of interest, the protein binding partner, the other protein binds.

47. The method of claim 44 wherein providing a cell comprises introducing nucleic acid that comprises a reporter gene downstream of a promoter that comprises a cis-acting element to which a fusion protein comprising the protein of interest, a fusion protein comprising the protein binding partner, or a fusion protein comprising the other protein binds.

48-65. (canceled)

66. The method according to claim 44 wherein expression of the protein of interest or the protein binding partner is operably under the control of an inducible promoter sequence such that the level of expression of that protein is capable of being modulated in the cell.

67. The method of claim 66 wherein the inducible promoter is a copper inducible promoter.

68. The method of claim 67 wherein the copper inducible promoter is the CUP1 promoter.

69. The method of claim 66 wherein the inducible promoter is a galactose-inducible promoter.

70. The method of claim 69 wherein the galactose-inducible promoter is the GAL1 promoter.

71. The method according to claim 44 wherein the counter-selectable reporter gene is operably connected to an inducible promoter such that the level of expression of said counter-selectable reporter gene is capable of being modulated in the cell.

72. The method of claim 71 wherein the inducible promoter is a copper inducible promoter.

73. The method of claim 72 wherein the copper inducible promoter is the CUP1 promoter.

74. The method of claim 71 wherein the inducible promoter is a galactose-inducible promoter.

75. The method of claim 74 wherein the galactose-inducible promoter is the GAL1 promoter.

76. The method of claim 71 wherein the inducible promoter is a phosphate regulatable promoter.

77. The method of claim 76 wherein the phosphate regulatable promoter is the PHO5 promoter.

78. The method of claim 44 wherein the counter selectable reporter gene is selected from the group consisting of URA 3, CYH2 and LYS2.

79. The method of claim 44 wherein the reporter gene at (i)(b) is selected from the group consisting of tet^{r}, Amp^{r}, Rif^{r}, bsdf^{r}, zeof^{r}, Kan^{r}, gfp, cobA, LacZ, CYH2, TRP1, LYS2, HIS3, HIS5, LEU2, URA3, ADE2, MET13 and MET15.

80. The method of claim 44 wherein the reporter genes bind different proteins via different cis-acting elements.

81. The method of claim 44 wherein the cis-acting elements are the same.

82. The method of claim 44 wherein one or more cis-acting elements is selected from a LexA operator, cI, and GAL4 recognition sequence.

83. The method of claim 82 wherein each cis-acting element binds to one or more DNA binding domains selected from the group consisting of a LexA DNA binding protein domain, cI protein domain and GAL4 protein domain, and wherein said DNA binding domain is present in a fusion protein comprising the binding partner protein and/or the other protein.

84. The method according to claim 44 to wherein one or more of the reporter genes encodes a detectable protein.

85. The method of claim 84 wherein the detectable protein is a fluorescent protein.

86. The method of claim 85 wherein the fluorescent protein is a green fluorescent protein (GFP) or luciferase protein or a product of the cobA gene.

87. The method of claim 84 wherein the detectable protein is detected colorimetrically.

88. The method of claim 87 wherein the detectable protein is a lacZ protein or β-galactosidase.

89. The method of claim 84 wherein the detectable protein is detected immunologically by antibody binding to the protein.

90. The method of claim 89 wherein the detectable protein is FLAG.

91. The method of claim 84 wherein the detectable protein is detected enzymatically.

92-107. (canceled)

108. The method of claim 44 wherein one or more nucleic acids encoding a fusion protein is in an expression vector.

109. The method of claim 108 further comprising introducing nucleic acid encoding one or more fusion proteins into an expression vector.

110. The method of claim 108 wherein the expression vector is selected from the group consisting of pDEATH-Trp, (SEQ ID NO: 10), pJFK (SEQ ID NO: 11), pDD (SEQ ID NO: 12), pRT2 (SEQ ID NO: 13), pGMS19 (SEQ ID NO: 15) and pDR10 (SEQ ID NO: 16).

111. The method of claim 108 wherein the expression vector is pGILDA.

112-122. (canceled)

123. A process for determining an inhibitor of an interaction between a protein of interest and a protein binding partner in a cell, said method comprising:
(i) performing the method according to claim 1 to thereby identify a mutation within a region in a protein of interest that mediates the ability of the protein to bind to a binding partner protein;
(ii) determining a fragment of the mutated form of the protein of interest said fragment comprising the region that mediates the ability of the protein to bind to the binding partner protein; and
determining a fragment in the native form of the protein of interest that is functionally equivalent to the fragment at (ii) wherein said fragment inhibits the interaction between the native form of the protein of interest and the binding partner.

124. The process of claim 123 comprising recovering a fragment in the native form of the protein of interest having an amino acid sequence that encompasses all or part of the mutated site in the mutated form of the protein of interest.

125. The process of claim 123 comprising synthesizing a fragment in the native form of the protein of interest having an amino acid sequence that encompasses all or part of the mutated site in the mutated form of the protein of interest.

126. The process of claim 124 wherein the fragment is no more than about 50 amino acid residues in length.

127. A process for determining or validating a protein interaction as a therapeutic drug target or validation reagent comprising:
(i) performing the process according to claim 123 thereby determining a fragment in a protein of interest that inhibits the interaction between the protein of interest and a binding partner protein; and
(ii) expressing the fragment in a cell or organism as a dominant negative inhibitor and determining a phenotype of the cell or organism that is modulated by the target protein or target nucleic acid wherein a modified phenotype of the cell or organism indicates that the protein interaction is a therapeutic target or validation reagent.

128. A process for determining or validating a protein interaction as a therapeutic drug target or validation reagent comprising:
(i) performing the method according to claim 1 to thereby identify a mutation within a region in a protein of interest that mediates the ability of a protein of interest to bind to a binding partner protein; and
(ii) expressing nucleic acid encoding the mutated form of the protein of interest in a model organism to thereby produce a knock-in of the mutant allele; and
(iii) detecting the phenotype of that mutant wherein a modified phenotype of the cell or organism indicates that the protein interaction is a therapeutic target or validation reagent.

129. A process for identifying a therapeutic or prophylactic compound comprising:
(i) performing the process according to claim 123 to thereby determine a fragment in a protein of interest that inhibits the interaction between the protein of interest and a binding partner protein; and
(ii) identifying a compound having the inhibitory activity of the fragment.

130. The process of claim 129 further comprising:
(a) optionally, determining the structure of the compound or modulator; and
(b) providing the compound or modulator or the name or structure of the compound or modulator such as, for example, in a paper form, machine-readable form, or computer-readable form.

131. The process of claim 129 further producing or synthesizing the compound.
